(19) United States
(12) Patent Application Publication
(10) Pub. No.: US 2024/0327460 A1
(43) Pub. Date: Oct. 3, 2024

(54) NORA INHIBITORS AND METHODS OF USE

(71) Applicant: NEW YORK UNIVERSITY, New York, NY (US)

(72) Inventors: Shohei KOIDE, New York, NY (US); Da-Neng WANG, New York, NY (US); Nathaniel TRAASETH, New York, NY (US); Akiko KOIDE, New York, NY (US); Douglas BRAWLEY, Jr., New York, NY (US); David SAUER, Reading (GB)

(21) Appl. No.: 18/294,021

(22) PCT Filed: Aug. 5, 2022
(86) PCT No.: PCT/US2022/039572

§ 371 Date: Jan. 31, 2024

Related U.S. Application Data
(60) Provisional application No. 63/229,918, filed on Aug. 5, 2021.

(51) Int. Cl. C07K 7/08; A61K 45/06; A61P 31/04; C07K 16/12; G01N 33/569

(52) U.S. Cl. CPC C07K 7/08; A61K 45/06; A61P 31/04; C07K 16/1271; G01N 33/56938; C07K 2317/565; C07K 2317/567; C07K 2317/569; C07K 2317/92; C07K 2318/00; G01N 2333/31; G01N 2800/26

(57) ABSTRACT

Disclosed herein are NorA peptide inhibitors, protein scaffolds including the NorA peptide inhibitor, and antibody-based molecules that bind NorA, as well as compositions containing the same. Also disclosed are combinations therapeutic agents that include an antibiotic and/or biocide; and a NorA inhibitor, protein scaffold, or antibody-based as disclosed herein. Use of these agents for treating a Staphylococcus aureus infection, potentiating the therapeutic efficacy of an antibiotic or biocide, diagnosing a S. aureus infection, and detecting NorA in a non-clinical biological sample are also disclosed.

This application claims the priority benefit of U.S. Provisional Patent Application Ser. No. 63/229,918, filed Aug. 5, 2021, which is hereby incorporated by reference in its entirety.

This invention was made with government support under RO1 AI108889, RO1 GM121994, RO1 NS108151, RO1 DK099023, and RO1 AI165782 awarded by the National Institutes of Health, and MCB 1902449 awarded by the National Science Foundation. The government has certain rights in the invention.

### FIELD

The invention generally relates to peptide-based inhibitors of the multidrug efflux pump, NorA, including antibodies and antibody binding fragments as well as antibody mimics and peptides, compositions and combination therapies containing the same, and the therapeutic, prophylactic, and diagnostic uses thereof.

### BACKGROUND

Widespread antibiotic resistance among hospital and community associated methicillin-resistant Staphylococcus aureus (MRSA), coupled with the lack of new antibiotics, poses an urgent threat to global public health (Antibiotic Resistance Threats in the United States, U.S. Department of Health and Human Services, Centers for Disease Control and Prevention, dx.doi.org/10.15620/cdc:82532, (Atlanta, GA) (2019)). Drug efflux is a first-line resistance mechanism used by MRSA to lower the intracellular antibiotic concentration (Nikaido, “Multidrug Resistance in Bacteria,” Annu Rev Biochem 78:119-146 (2009); Du et al., “Multidrug Efflux Pumps: Structure, Function and Regulation,” Nat Rev Microbiol 16:523-539 (2018); Piddock, “Multidrug-resistance Efflux Pumps—Not Just for Resistance,” Nat Rev Microbiol 4:629-636 (2006)), facilitating pathogen survival in the presence of antibiotics and the evolution of higher-level resistance mechanisms (Du et al., “Multidrug Efflux Pumps: Structure, Function and Regulation,” Nat Rev Microbiol 16:523-539 (2018); Singh et al.. “Temporal Interplay Between Efflux Pumps and Target Mutations in Development of Antibiotic Resistance in Escherichia coli,” Antimicrob Agents Chemother 56:1680-1685 (2012); Papkou et al., “Efflux Pump Activity Potentiates the Evolution of Antibiotic Resistance Across S. aureus Isolates,” Nat Commun 11:3970 (2020)).

NorA is a chromosomally encoded multidrug efflux pump belonging to the core genome of S. aureus and present in all MRSA strains (Costa et al., “Genetic Diversity of norA, Coding for a Main Efflux Pump of Staphylococcus aureus,” Front Genet 9:710 (2018)). As a member of the major facilitator superfamily (MFS) and the DHA12 subfamily (12-transmembrane domain drug:H⁺ antiporter), it couples drug extrusion to the proton motive force (Paulsen et al., “Proton-dependent Multidrug Efflux Systems,” Microbiol Rev 60:575-608 (1996); Yu et al., “NorA Functions as a Multidrug Efflux Protein in Both Cytoplasmic Membrane Vesicles and Reconstituted Proteoliposomes,” J Bacteriol 184:1370-1377 (2002); Ng et al., “Quinolone Resistance Mediated by norA: Physiologic Characterization and Relationship to flqB, a Quinolone Resistance Locus on the Staphylococcus aureus Chromosome,” Antimicrob Agents Chemother 38:1345-1355 (1994)). Overexpression of NorA through upregulating promoter mutations and/or chromosomal gene duplication (Papkou et al., “Efflux Pump Activity Potentiates the Evolution of Antibiotic Resistance Across S. aureus Isolates,” Nat Commun 11:3970 (2020); Kaatz et al., “Effect of Promoter Region Mutations and mgrA Overexpression on Transcription of norA, Which Encodes a Staphylococcus aureus Multidrug Efflux Transporter,” Antimicrob Agents Chemother 49:161-169 (2005); DeMarco et al., “Efflux-related Resistance to Norfloxacin, Dyes, and Biocides in Bloodstream Isolates of Staphylococcus aureus,” Antimicrob Agents Chemother 51:3235-3239 (2007)) confers resistance to hydrophilic fluoroquinolones (ciprofloxacin, enoxacin, norfloxacin, ofloxacin), chloramphenicol, and cationic dyes (Ng et al., “Quinolone Resistance Mediated by norA: Physiologic Characterization and Relationship to flqB, a Quinolone Resistance Locus on the Staphylococcus aureus Chromosome,” Antimicrob Agents Chemother 38:1345-1355 (1994); DeMarco et al., “Efflux-related Resistance to Norfloxacin, Dyes, and Biocides in Bloodstream Isolates of Staphylococcus aureus,” Antimicrob Agents Chemother 51:3235-3239 (2007); Yoshida et al., “Nucleotide Sequence and Characterization of the Staphylococcus aureus norA Gene, Which Confers Resistance to Quinolones,” J Bacteriol 172:6942-6949 (1990); Neyfakh et al., “Fluoroquinolone Resistance Protein NorA of Staphylococcus aureus is a Multidrug Efflux Transporter,” Antimicrob Agents Chemother 37:128-129 (1993); Kaatz et al., “Efflux-mediated Fluoroquinolone Resistance in Staphylococcus aureus,” Antimicrob Agents Chemother 37:1086-1094 (1993)). This fitness advantage provided by NorA to S. aureus also allows for the evolution of mutations to the antibiotic target (Papkou et al., “Efflux Pump Activity Potentiates the Evolution of Antibiotic Resistance Across S. aureus Isolates,” Nat Commun 11:3970 (2020)).

Despite NorA's biomedical relevance in S. aureus antibiotic resistance (Yu et al., “NorA Functions as a Multidrug Efflux Protein in Both Cytoplasmic Membrane Vesicles and Reconstituted Proteoliposomes,” J Bacteriol 184:1370-1377 (2002); Ng et al., “Quinolone Resistance Mediated by norA: Physiologic Characterization and Relationship to flqB, a Quinolone Resistance Locus on the Staphylococcus aureus Chromosome,” Antimicrob Agents Chemother 38:1345-1355 (1994); DeMarco et al., “Efflux-related Resistance to Norfloxacin, Dyes, and Biocides in Bloodstream Isolates of Staphylococcus aureus,” Antimicrob Agents Chemother 51:3235-3239 (2007); Neyfakh et al., “Fluoroquinolone Resistance Protein NorA of Staphylococcus aureus is a Multidrug Efflux Transporter,” Antimicrob Agents Chemother 37:128-129 (1993); Kaatz et al., “Efflux-mediated Fluoroquinolone Resistance in Staphylococcus aureus,” Antimicrob Agents Chemother 37:1086-1094 (1993)), there are no structural or functional data to explain the mechanisms underlying multidrug specificity or proton-coupled transport. Such information would provide key insight into the substrate binding pocket to aid in the design of potent and selective efflux pump inhibitors (EPIs). Combination treatments of EPIs with antibiotics are a promising therapeutic strategy for treating infections from pathogenic S. aureus strains and for reducing the emergence of higher-level resistance mechanisms (de Sousa Andrade et al., “Antimicrobial Activity and Inhibition of the NorA Efflux Pump of Staphylococcus aureus by Extract and Isolated Compounds From Arrabidaea brachypoda,” Microb Pathog 140:103935 (2020); Brincat et al., “Discovery of Novel Inhibitors of the NorA Multidrug Transporter of Staphylococcus aureus,” J. Med. Chem. 54:354-365 (2011)). However, development of EPIs has been hindered by systemic toxicity associated with off-target effects (Kathawala et al., “The Modulation of ABC Transporter-mediated Multidrug Resistance in Cancer: A Review of the Past Decade,” Drug Resist Updat 18:1-17 (2015); Renau et al., “Conformationally-restricted Analogues of Efflux Pump Inhibitors That Potentiate the Activity of Levofloxacin in Pseudomonas aeruginosa,” Bioorg. Med. Chem. Lett. 13:2755-2758 (2003)). To date, there are no clinically approved inhibitors targeting efflux pumps in S. aureus.

It would be desirable, therefore, to develop NorA inhibitors that can be used in combination with antibiotics rendered ineffective by NorA-mediated drug efflux to sensitize drug-resistant strains of bacteria, including S. aureus, to antibiotic treatment.

### SUMMARY

A first aspect relates to a NorA peptide inhibitor.

In one embodiment, the NorA peptide inhibitor comprises the amino acid sequence of X₁YYX₄X₅WRX₈X₉GX₁₁X₁₂ (SEQ ID NO:1), wherein X₁, X₄, X₅, X₈, X₉, X₁₁, X₁₂ are selected from any amino acid residue, or more particularly wherein X₁ is G or Y, X₄ is P or Y, X₅ is Y or A, X₈ is M or V, X₉ is Y or G, X₁₁ is F or Y and X₁₂ is Y or W. Exemplary NorA peptide inhibitors include those of SEQ ID NOS: 2-7.

In another embodiment, the NorA peptide inhibitor comprises the amino acid sequence of X₁X₂X₃X₄X₅WRX₈X₉X₁₀X₁₁X₁₂ (SEQ ID NO: 27) wherein X₁ is Y, I, or V; X₂ is Y, F, or W; X₃ is Y, F, I, L, M, V, or W; X₄ is Y or W; X₅ is A, L, S, or V; X₈ is V, A, D, F, G, H, I, L, M, R, W, or Y; X₉ is G, F, P, S, or T; X₁₀ is G, A, F, I, L, N, Q, S, T, V, or Y; X₁₁ is Y, A, C, F, H, I, L, M, R, S, T, V, or W; and X₁₂ is W, F, L, or Y.

A second aspect relates to a protein scaffold comprising the NorA peptide inhibitor according to the first aspect.

A third aspect relates to an antibody-based molecule that binds NorA, said antibody-based molecule comprising a heavy chain variable region that comprises (i) a complementarity-determining region 1 (CDR-H1) comprising the amino acid sequence of any one of SEQ ID NOS: 12-14 or a modified amino acid sequence having at least 80% sequence identity to any one of SEQ ID NOS: 12-14; (ii) a complementarity-determining region 2 (CDR-H2) comprising the amino acid sequence of any one of SEQ ID NOS: 15-20 or a modified amino acid sequence having at least 80% sequence identity to any one of SEQ ID NOS: 15-20; and/or (iii) a complementarity-determining region 3 (CDR-H3) comprising the amino acid sequence of any one of SEQ ID NOS: 5-11 or a modified amino acid sequence having at least 80% sequence identity to any one of SEQ ID NOs: 5-11.

The third aspect also relates to an antibody-based molecule further comprising a light chain variable region that comprises: (i) a complementarity-determining region 1 (CDR-L1) having the amino acid sequence of SEQ ID NO: 21 or a modified amino acid sequence having at least 80% sequence identity to SEQ ID NO: 21; (ii) a complementarity-determining region 2 (CDR-L2) having the amino acid sequence of SEQ ID NO: 22, or a modified amino acid sequence having at least 80% sequence identity to SEQ ID NO: 22; and/or (iii) a complementarity-determining region 3 (CDR-L3) having the amino acid sequence of any one of SEQ ID NOS: 23-26 or a modified amino acid sequence having at least 80% sequence identity to any one of SEQ ID NOS: 23-26.

A fourth aspect relates to an isolated polynucleotide encoding the NorA peptide inhibitor according to the first aspect, the protein scaffold according to the second aspect, or the antibody-based molecule according to the third aspect.

A fifth aspect relates to a vector comprising the isolated polynucleotide according to the fourth aspect.

A sixth aspect relates to a delivery vehicle comprising the isolated polynucleotide according to the fourth aspect or the vector according to the fifth aspect.

A seventh aspect relates to a host cell comprising the vector according to the fifth aspect.

An eighth aspect relates to a pharmaceutical composition comprising the NorA peptide inhibitor according to the first aspect, the protein scaffold according to the second aspect, the antibody-based molecule that binds NorA according to the third aspect, the polynucleotide according to the fourth aspect, the vector according to the fifth aspect, or the delivery vehicle according to the sixth aspect; and a pharmaceutically acceptable carrier.

A ninth aspect relates to a combination therapeutic comprising an antibiotic and/or biocide; and a NorA inhibitor selected from the NorA peptide inhibitor according to the first aspect, the protein scaffold according to the second aspect, and the antibody-based molecule that binds NorA according to the third aspect.

A tenth aspect relates to a method of treating a Staphylococcus aureus infection in a subject, said method comprising administering, to a subject having a S. aureus infection, an antibiotic or biocide and the pharmaceutical composition according to the eighth aspect, wherein said pharmaceutical composition is administered in an amount effective to treat the S. aureus infection in the subject.

An eleventh aspect relates to a method of potentiating the therapeutic efficacy of an antibiotic or biocide in a subject having a Staphylococcus aureus infection, said method comprising administering, to a subject having a S. aureus infection and exhibiting NorA mediated antibiotic or biocide resistance, an antibiotic or biocide and the pharmaceutical composition according to the eighth aspect, wherein the pharmaceutical compositions is administered in an amount effective to potentiate the therapeutic efficacy of the antibiotic or biocide in the subject.

A twelfth aspect relates to a method of diagnosing a S. aureus infection in a subject, said method comprising contacting a sample from a subject having a S. aureus infection with the NorA peptide inhibitor according to the first aspect, the protein scaffold according to the second aspect, or the antibody-based molecule that binds NorA according to the third aspect, under conditions effective to detect NorA expression in the sample; detecting NorA expression in said sample based on said contacting; and diagnosing the S. aureus infection in the subject based on said detecting.

A thirteenth aspect relates to a method of detecting NorA in a non-clinical biological sample, said method comprising contacting the sample with the NorA peptide inhibitor according to the first aspect, the protein scaffold according to the second aspect, or the antibody-based molecule that binds NorA according to the third aspect, under conditions effective to detect NorA expression in the sample, and detecting NorA expression in said sample based on said contacting.

As demonstrated in the accompanying examples, using cryo-electron microscopy (cryo-EM) and in vivo drug resistance assays, the examples provide insight into how NorA confers multidrug resistance to S. aureus. Characteristics of the transporter's structure, function, and mechanism of action are described. Importantly, synthetic antigen-binding fragments (Fabs) that bind to the extracellular side of NorA and inhibit NorA-mediated antibiotic efflux are disclosed. The use of these NorA-binding fragments and peptide-based derivatives thereof in combination with one or more with antibiotic agents as a therapeutic treatment for existing bacterial infection or as a prophylactic to prevent bacterial infection is therefore contemplated.

### BRIEF DESCRIPTION OF THE DRAWINGS

FIGS. 1A-D illustrate NorA-Fab complex structures determined using single particle cryo-EM. NorA structures were solved in complex with Fab25 at 3.74 Å (FIG. 1A) and Fab36 at 3.16 Å resolution (FIG. 1B) using cryo-EM. The light and heavy chains of Fab25 are shown in light and dark grey (or grey and pink in color version), while those of Fab36 are shown in light and dark grey (or grey and light blue in color version), respectively. The transmembrane helices of NorA are generally designated (rainbow colored in color version). TM1-6 and TM7-12 define the N- and C-domains of the transporter, respectively. FIGS. 1C-D afford expanded views of CDRH3 interactions for Fab25 (FIG. 1C) and Fab36 (FIG. 1D) in the substrate binding pocket of NorA. Distances (in A) are displayed as dotted black lines. The partial primary sequences of CDRH3 for Fab25 (residues 1-18 of SEQ ID NO: 6) and Fab36 (residues 1-18 of SEQ ID NO: 7) are shown between FIGS. 1C-D.

FIGS. 2A-D identify key residues within NorA essential for drug resistance. In FIG. 2A, electrostatic surface representations of NorA, where dark gray color (red and blue, in color version) designates negatively and positively charged sites. The left and middle views are depicted in a similar orientation as FIGS. 1A-B. The left and right panels display slices of the binding pocket illustrating the anionic patch formed by Glu222 and Asp307. The right panel is rotated by 900 relative to the left and center panels, and depicts NorA from the extracellular side of the membrane, looking into the outward-open substrate binding pocket. FIG. 2B shows Norfloxacin MIC values in MRSA obtained for the wild-type strain (MRSA) or a strain with the native NorA gene disrupted by a transposon insertion (MRSA^{ΔnorA}). NorA mutants denoted under the MRSA^{ΔnorA} dotted line were expressed from a hemin-inducible plasmid, where NorA corresponds to the wild-type sequence. The number of independent MIC experiments (n=2-5) corresponds to the number of grey circles superimposed on the bar graph for each sample; data are presented as mean values+/−SEM among the independent experiments. The MIC upper limit of detection was 256 μg/ml; mutants with reported MIC values at 256 μg/ml may have a true MIC that exceed this value. P-values were calculated using an unpaired, two-tailed t-test for each single-site mutant relative to MRSA^{ΔnorA}+NorA: **** P<0.0001, *** P<0.001, ** P<0.01, * P=0.0158. FIG. 2C shows MIC values for NorA mutants displayed on the structure in a heat map representation. NorA is shown in a similar orientation as depicted in the right panel of FIG. 2A. The heat map follows a linear scale, where dark gray (green in color version) corresponds to loss-of-function and light gray (yellow in color version) signifies no change in function relative to MRSA^{ΔnorA}+NorA. Circled numbers denote the TM helices of NorA. FIG. 2D is a sequence homology analysis of seven DHA12-family MFS drug transporters showing a portion of TM7 and TM10, which contain Glu222 and Asp307 in NorA, respectively. NorA TM7 (SEQ ID NO: 42) and TM10 (SEQ ID NO: 43); Bmr TM7 (SEQ ID NO: 44) and TM10 (SEQ ID NO: 45); Blt TM7 (SEQ ID NO: 46) and TM10 (SEQ ID NO: 47); LmrP TM7 (SEQ ID NO: 48) and TM10 (SEQ ID NO: 49); EmrD TM7 (SEQ ID NO: 50) and TM10 (SEQ ID NO: 51); MdfA TM7 (SEQ ID NO: 52) and TM10 (SEQ ID NO: 53); YajR TM7 (SEQ ID NO: 54) and TM10 (SEQ ID NO: 55). Anionic residues (Asp, D; Glu, E) are highlighted (red in color version) and cationic residues (Arg, R) are highlighted (blue in color version).

FIGS. 3A-D illustrate Fab binding and inhibition of NorA. FIG. 3A is a schematic of E. coli co-expression assay involving separate inducible promoters for norA and fab genes encoded on plasmids (circles). NorA (light grey; surface representation) folds in the inner membrane and the Fab (dark grey, blue in color version; surface representation) is secreted to the periplasm. Efflux of norfloxacin (solid circles, green in color version) is mediated by NorA in the absence of Fab (arrow; green in color version), whereas efflux is inhibited when NorA is bound to Fab (“X”, red in color version). FIG. 3B shows growth inhibition experiments of E. coli co-expressing NorA and Fab36 (left) or Fab_{control} (right) in the presence of norfloxacin and arabinose (inducer of Fabs; abbreviated “ara”). For clarity, only a few datasets at select arabinose concentrations are displayed. Solid lines and shading in the same color correspond to the average and standard deviation of four independent experiments, respectively, with each independent experiment performed with two technical replicates. P-values were calculated using an unpaired, two-tailed t-test with Welch's correction for the 24 hr timepoint and indicate significance relative to the 0% arabinose concentration: **** P<0.0001, * P=0.0305. FIG. 3C shows growth inhibition at the 24 hr timepoint of E. coli co-expressing NorA and Fab36 (left) or Fab_{control} (right) as a function of arabinose concentration. Bar graphs indicate the average and standard deviation among four independent experiments. Black circles correspond to all replicate datapoints from the independent experiments. P-values are the same as described for FIG. 3B. FIG. 3D shows immunoblot (IB) analyses probing for NorA and Fab expression at 24 hr and corresponding to the arabinose concentrations in FIG. 3C. Analyses were performed from growths in the absence of norfloxacin to normalize against growth inhibition variation when norfloxacin was present. Similar results were obtained in two independent experiments.

FIGS. 4A-G illustrate NorA binding and MRSA growth inhibition by a peptide mimicking CDRH3. FIGS. 4A, 4C show growth inhibition experiments in MRSA in the presence of norfloxacin (12.5 μg/ml) and varying concentrations of NPI-1 (SEQ ID NO: 3) (FIG. 4A) or NPI-2 (SEQ ID NO: 56) (FIG. 4C). Solid lines and shading in the same color correspond to the average and standard deviation of three independent experiments, respectively. P-values were calculated using an unpaired, two-tailed t-test for the 10 hr timepoint and indicate significance relative to the vehicle: **** P<0.0001, *** P<0.001. FIG. 4B shows growth inhibition experiments in MRSA at the 10 hr timepoint as a function of peptide concentration in the presence of 12.5 μg/ml norfloxacin. Data are presented as mean values+/−SEM among three independent experiments. The fitted curve was used to determine the IC₅₀ for NPI-1 (0.72±0.08 μM). FIG. 4D illustrates a representative fluorescence polarization (FP) experiment using FITC-NPI-1 in the presence of varying NorA concentrations. The solid line reflects the best fit to the data. The reported K_{d} value and error reflects the average and standard deviation from seven independent experiments. FIG. 4E illustrates a representative competitive FP binding experiment with a fixed concentrations of NorA and FITC-NPI-1 and a variable concentration of norfloxacin. The control experiment has the same amount of FITC-NPI-1 in the absence of NorA. Three replicate data points in grey circles are superimposed on the bar graph; data are presented as mean values+/−SEM among the replicates. A P-value was calculated using an unpaired, two-tailed t-test with Welch's correction for the “+NorA” dataset and indicates significance between the two norfloxacin concentrations: ** P=0.006. Similar results were obtained in four independent experiments. FIG. 4F is a competitive MST binding experiment with fixed concentrations of NorA and fluorescently labelled Fab36 and a variable concentration of NPI-1. Data are presented as mean values+/−SEM among three replicates. The line on the plot was calculated using the K_{d} value determined for FITC-NPI-1 from the FP experiments in FIG. 4D (see infra, Methods section of Examples). FIG. 4G is a cartoon illustrating the strategy for MRSA growth inhibition through the addition of NPI-1 and antibiotic. The peptide NPI-1 was designed based on the portion of the Fab CDRH3 loop that inserts within the substrate binding pocket of NorA (step 1). When exogenously administered to MRSA, NPI-1 diffuses through the peptidoglycan layer, binds NorA, and inhibits antibiotic efflux (step 2). In turn, this raises the intracellular antibiotic concentration (step 3) and leads to MRSA growth arrest.

FIGS. 5A-5E illustrate the preparation of NorA samples for Fab screening and cryo-EM. FIG. 5A shows the purification of NorA in LMNG detergent. On the left side of FIG. 5A is a SEC chromatogram displaying absorbance at 280 nm (Abs₂₉₀ₙₘ) for NorA in LMNG. On the right side of FIG. 5A is a coomassie-stained SDS-PAGE gel of peak fractions from the SEC chromatogram. Similar results were obtained in ˜15 independent experiments. FIG. 5B is a coomassie-stained SDS-PAGE gel showing successful reconstitution of NorA (His-tagged) into biotinylated membrane scaffold protein (MSP) nanodiscs for phage display screening of Fabs. The sample was passed over a Ni-NTA column with the imidazole elution displaying both NorA and MSP bands. Similar reconstitution results were obtained in three independent experiments. FIG. 5C are SEC chromatograms of NorA in PMAL-C8 amphipol alone (black traces) and NorA in the presence of three-fold molar excess of Fab25 (top, magenta) or Fab36 (bottom, blue). Major fractions from the left-shifted peaks indicated by arrows were collected and applied onto cryo-EM grids for structure determination. FIG. 5D and FIG. 5E are MST binding curves of fluorescently labelled Fabs to NorA reconstituted in PMAL-C8 amphipol. K_{d} values are shown next to each dataset; ‘n.d.’ refers to not determined. The error in K_{d} values for Fab25 (1.2±0.7 μM) and Fab36 (140±20 nM) represent the average and standard deviation of two independent runs with three or four replicates per independent run. The error range in the K_{d} value for Fab36^{W133A} (2 to 4 μM) corresponds to the 95% confidence interval of the non-linear fit. The Fab36^{R134A} binding curve could not be accurately fit and the K_{d} value was estimated to be greater than the highest concentration of NorA in the experiment (>14 μM). MST binding experiments with Fab36^{W133A} and Fab36^{R134A} were collected with three replicates each.

FIGS. 6A-6E show the expression and function of NorA mutants in MRSA and E. coli. FIG. 6A is a dilution experiment in MRSA and MRSA^{ΔnorA} strains to test bacteriostatic or bactericidal effect of norfloxacin. Cells were grown overnight to saturation, diluted 1,000-fold into fresh media containing norfloxacin at 0, 12.5, or 25 μg/ml, grown in liquid culture for 1, 2, or 4-h, and plated on solid media for determination of colony forming units (CFUs). The fold change refers to CFUs at the 1, 2, and 4-h time points relative to Time=0-h, which did not contain norfloxacin. Data are presented as mean values±s.d. among three replicates. FIG. 6B shows immunoblot analysis of the membrane fraction after induction of MRSA^{ΔnorA} or MRSA^{ΔnorA} transformed with a hemin-inducible plasmid encoding wild-type NorA and NorA mutants. The samples were immunoblotted for NorA and SrtA (control membrane protein). Similar results were obtained in two immunoblots. FIG. 6C shows growth inhibition experiments using the MRSA^{ΔnorA} strain transformed with a hemin-inducible plasmid encoding NorA or NorA mutants. NorA expression was induced with 1 μM hemin and carried out in the presence or absence of 12.5 μg/mL norfloxacin. Solid lines and shading in the same color correspond to the average and standard deviation of four technical replicates from one representative experiment, respectively. Similar results were verified in at least two independent experiments. P-values were calculated using an unpaired, two-tailed t-test for each single-site mutant relative to MRSA^{ΔnorA}+NorA at the 10-h timepoint: ****P<0.0001, ** P=0.0045. On the top of FIG. 6D is a representative serial dilution experiment on LB agar using E. coli transformed with a plasmid encoding NorA, NorA mutants, or no construct (vector) in the presence of norfloxacin (15 nM). On the bottom of FIG. 6D are the results of serial dilution experiments for 20 single-site NorA mutants at aspartate and glutamate positions. Three mutants showed ablated resistance phenotypes toward norfloxacin (D63A, E222A, and D307A). The serial dilution screen was performed one time; loss-of-function mutants identified from the screen were confirmed in independent E. coli serial dilution experiments and in MRSA^{ΔnorA} using MIC and growth inhibition experiments. FIG. 6E displays representative images from norfloxacin MIC experiments using MRSA^{ΔnorA} strains transformed with a hemin-inducible plasmid encoding NorA (top left) or D63A (top right). NorA expression was induced with 1 μM hemin. The MIC values were read at the intersection of the inhibition eclipse and the strip (units of μg/ml). On the bottom of FIG. 6E is a plot of norfloxacin MIC values for MRSA or MRSA^{ΔnorA} transformed with plasmids encoding wild-type NorA or NorA mutants. The MIC upper limit of detection was 256 μg/ml. P-values were calculated using an unpaired, two-tailed t-test for each single-site mutant relative to MRSA^{ΔnorA}+NorA: ****P<0.0001. The number of independent MIC experiments (n=2-5) correspond to the number of black circles superimposed on the bar graph for each sample; data are presented as mean values±s.d. among the independent experiments.

FIGS. 7A-7E illustrate how Fab inhibition of NorA is assessed through an E. coli co-expression assay. FIG. 7A shows a growth inhibition experiment of E. coli co-expressing NorA and Fabcontrol, Fab25, or Fab36. Experiments were performed in the presence (left) or absence (right) of 1.2 μg/mL norfloxacin and arabinose (inducer of Fabs; abbreviated ‘ara’). For clarity, only a few datasets at select arabinose concentrations are displayed. Solid lines and shading in the same color correspond to the average and standard deviation of four independent experiments, respectively. P-values were calculated using an unpaired, two-tailed t-test with Welch's correction for the 24-h timepoint and indicate significance relative to the 0% arabinose concentration: **** P<0.0001, ** P=0.0013, * P=0.0305. FIG. 7B is an analysis of the growth inhibition data at the 24-h timepoint for E. coli co-expressing NorA and Fab_{control}, Fab25, or Fab36 as a function of arabinose concentration. Bar graphs indicate mean values±s.d. among four independent experiments, each with two replicates per experiment. Black circles correspond to all replicate datapoints from the independent experiments. FIG. 7C shows immunoblot analyses of E. coli co-expressing NorA and Fab_{control}, Fab25, or Fab36 after 24-h of growth and in the presence of varying arabinose concentration. Analyses were performed from growths in the absence of norfloxacin to normalize against growth inhibition variation when norfloxacin was present. Similar results were obtained in two immunoblots. FIG. 7D are normalized E. coli growth curves calculated by dividing the OD_{600nm,24-h} value in the presence of norfloxacin by the corresponding value in the absence of norfloxacin for each arabinose concentration. Data are presented as mean values±s.d. among four independent experiments. P-values were calculated using an unpaired, two-tailed t-test with Welch's correction for the highest arabinose concentration for the NorA+Fab25 and NorA+Fab36 samples relative to the NorA+Fab_{control} sample: **** P<0.0001, ** P=0.0084. FIG. 7E displays immunoblot analysis of a NorA pull-down experiment for E. coli co-expressing NorA and Fab_{control}, Fab25, or Fab36 with 0.2% arabinose. Following co-expression, cells were lysed and NorA was purified from the membrane fraction using affinity chromatography. Elution fractions were immunoblotted for NorA and Fab. Similar results were obtained in two immunoblots.

FIGS. 8A-8C illustrate the inhibition of MRSA growth by combination treatment of norfloxacin and a CDRH3 mimicking peptide targeting NorA. FIGS. 8A and 8B show growth inhibition of MRSA or MRSA^{ΔnorA} treated with varying concentrations of NPI-1 or NPI-2, respectively, in the presence or absence of norfloxacin (12.5 μg/ml). The peptide was added at the start of the time course (Time=0-h). Solid lines and shading in the same color correspond to the average and standard deviation of three independent experiments, respectively. FIG. 8C shows growth inhibition of MRSA or MRSA^{ΔnorA} at the 10-h timepoint from FIGS. 8A-8B plotted against the NPI-1 (left) or NPI-2 (right) concentration, respectively. Data are presented as mean values±s.d. among three independent experiments. The fitted curve was used to determine the IC₅₀ for NPI-1 (0.72±0.08 μM).

### DETAILED DESCRIPTION

### General Definitions

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2nd ed., Cold Spring Harbor Press, Plainsview, New York (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms “a” “an”, and “the” include both singular and plural referents unless the context clearly dictates otherwise.

The terms “comprising”, “comprises” and “comprised of” as used herein are synonymous with ‘including’, ‘includes’ or ‘containing’, ‘contains’, and are inclusive or open-ended and do not exclude additional, non-recited members, compounds, products, elements or method steps. The expression “essentially consists of” used in the context of a product or a composition (“a product essentially consisting of” or “a composition essentially consisting of”) means that additional molecules may be present but that such molecule does not change/alter the characteristic/activity/functionality of said product or composition. For example, a composition may essentially consist of an antibody or an antibody fragment if the composition as such would exhibit similar characteristic/activity/functionality as one of the antibody or as the one of the antibody fragments.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

As used herein, amino acid residues will be indicated either by their full name or according to the standard three-letter or one-letter amino acid code.

As used herein, the terms “polypeptide” or “protein” are used interchangeably, and refer to a polymeric form of amino acids of any length, which can include coded and non-coded amino acids, chemically or biochemically modified or derivatized amino acids, and polypeptides having modified peptide backbones. A “peptide” is also a polymer of amino acids with a length which is usually of up to 50 amino acids. A polypeptide or peptide is represented by an amino acid sequence.

As used herein, the terms “nucleic acid molecule”, “polynucleotide”, “polynucleic acid”, “nucleic acid” are used interchangeably and refer to polymeric form of nucleotides of any length, either deoxyribonucleotides or ribonucleotides, or analogs thereof. A nucleic acid molecule is represented by a nucleic acid sequence, which is primarily characterized by its base sequence. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term “homology” denotes at least secondary structural identity or similarity between two macromolecules, particularly between two polypeptides or polynucleotides, from same or different taxons, wherein said similarity is due to shared ancestry. Hence, the term ‘homologues’ denotes so-related macromolecules having said secondary and optionally tertiary structural similarity. For comparing two or more nucleotide sequences, the ‘(percentage of) sequence identity’ between a first nucleotide sequence and a second nucleotide sequence may be calculated using methods known by the person skilled in the art, e.g. by dividing the number of nucleotides in the first nucleotide sequence that are identical to the nucleotides at the corresponding positions in the second nucleotide sequence by the total number of nucleotides in the first nucleotide sequence and multiplying by 100% or by using a known computer algorithm for sequence alignment such as NCBI Blast. In determining the degree of sequence similarity between two amino acid sequences, the skilled person may take into account so-called ‘conservative’ amino acid substitutions, which can generally be described as amino acid substitutions in which an amino acid residue is replaced with another amino acid residue of similar chemical structure and which has little or essentially no influence on the function, activity or other biological properties of the polypeptide. Possible conservative amino acid substitutions have been already exemplified herein. Amino acid sequences and nucleic acid sequences are said to be ‘exactly the same’ if they have 100% sequence identity over their entire length.

Throughout this application, each time one refers to a specific amino acid sequence SEQ ID NO (take SEQ ID NO: Y as example), one may replace it by: a polypeptide comprising an amino acid sequence that has at least 80% sequence identity or similarity with amino acid sequence SEQ ID NO: Y. Throughout this application, the wording “a sequence is at least X % identical with another sequence” may be replaced by “a sequence has at least X % sequence identity with another sequence”.

Each amino acid sequence described herein by virtue of its identity percentage (at least 80%) with a given amino acid sequence respectively has in a further preferred embodiment an identity of at least 80%, 81%, 82%, 83%, 84%, 85%, 86%, 87%, 88%, 89%, 90%, 91%, 92%, 93%, 94%, 95%, 96%, 97%, 98%, 99% or more identity with the given amino acid sequence respectively. In a preferred embodiment, sequence identity is determined by comparing the whole length of the sequences as identified herein. Each amino acid sequence described herein by virtue of its similarity percentage (at least 80%) with a given amino acid sequence respectively has in a further preferred embodiment a similarity of at least 80%, 81%, 82%, 83%, 84%, 85%, 86%, 87%, 88%, 89%, 90%, 91%, 92%, 93%, 94%, 95%, 96%, 97%, 98%, 99% or more similarity with the given amino acid sequence respectively. In a preferred embodiment, sequence similarity is determined by comparing the whole length of the sequences as identified herein. Unless otherwise indicated herein, identity or similarity with a given SEQ ID NO means identity or similarity based on the full length of said sequence (i.e. over its whole length or as a whole).

“Sequence identity” is herein defined as a relationship between two or more amino acid (polypeptide or protein) sequences or two or more nucleic acid (polynucleotide) sequences, as determined by comparing the sequences. The identity between two amino acid sequences is preferably defined by assessing their identity within a whole SEQ ID NO as identified herein or part thereof. Part thereof may mean at least 50% of the length of the SEQ ID NO, or at least 60%, or at least 70%, or at least 80%, or at least 90%.

In the art, “identity” also means the degree of sequence relatedness between amino acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, FASTA, BLASTN, and BLASTP (Altschul, S. F. et al., J. Mol. Biol. 215:403-410 (1990)), EMBOSS Needle (Madeira, F., et al., Nucleic Acids Research 47(W1): W636-W641 (2019)). The BLAST program is publicly available from NCBI and other sources (BLAST Manual, Altschul, S., et al., NCBI NLM NIH Bethesda, MD 20894; Altschul, S., et al., J. Mol. Biol. 215:403-410 (1990)). The EMBOSS program is publicly available from EMBL-EBI. The well-known Smith Waterman algorithm may also be used to determine identity. The EMBOSS Needle program is the preferred program used.

Preferred parameters for polypeptide sequence comparison include the following: Algorithm: Needleman and Wunsch, J. Mol. Biol. 48 (3):443-453 (1970); Comparison matrix: BLOSUM62 from Henikoff and Henikoff, Proc. Natl. Acad. Sci. USA. 89:10915-10919 (1992); Gap Open Penalty: 10; and Gap Extend Penalty: 0.5. A program useful with these parameters is publicly available as the EMBOSS Needle program from EMBL-EBI. The aforementioned parameters are the default parameters for a Global Pairwise Sequence alignment of proteins (along with no penalty for end gaps).

Preferred parameters for nucleic acid comparison include the following: Algorithm: Needleman and Wunsch, J Mol. Biol. 48:443-453 (1970); Comparison matrix: DNAfull; Gap Open Penalty: 10; Gap Extend Penalty: 0.5. A program useful with these parameters is publicly available as the EMBOSS Needle program from EMBL-EBI. The aforementioned parameters are the default parameters for a Global Pairwise Sequence alignment of nucleotide sequences (along with no penalty for end gaps).

Also provided herein are embodiments wherein any embodiment described herein may be combined with any one or more other embodiments, provided the combination is not mutually exclusive.

### NorA Peptide Inhibitors

NorA is a chromosomally encoded multidrug efflux pump belonging to the core genome of Staphylococcus aureus and is present in all methicillin resistant S. aureus (MRSA) strains. Overexpression of NorA through upregulating promoter mutations and/or chromosomal gene duplication confers resistance to hydrophilic fluoroquinolones (ciprofloxacin, enoxacin, norfloxacin, ofloxacin), chloramphenicol, and cationic dyes by coupling drug efflux to the proton motive force. Accordingly, inhibition of NorA offers a means for preventing and/or reversing antibiotic resistance of S. aureus strain. Thus, the present disclosure relates to NorA inhibitors and antibody-based molecules, pharmaceutical compositions comprising these inhibitor and antibodies, and methods of treatment and diagnosis utilizing these inhibitors and antibody-based molecules.

One aspect of the disclosure relates to a NorA peptide inhibitor that comprises the amino acid sequence of X₁YYX₄X₅WRX₈X₉GX₁₁X₁₂ (SEQ ID NO:1), wherein X₁, X₄, X₅, X₈, X₉, X₁₁, X₁₂ are selected from any amino acid residue.

In any embodiment, the NorA peptide inhibitor comprising the amino acid sequence of X₁YYX₄X₅WRX₈X₉GX₁₁X₁₂ (SEQ ID NO:1), wherein X₁ is G or Y, X₄ is P or Y, X₅ is Y or A, X₈ is M or V, X₉ is Y or G, X₁₁ is F or Y and X₁₂ is Y or W.

In one embodiment, an exemplary NorA peptide inhibitor of the present disclosure comprises the amino acid sequence of YYYYAWRVGGYW (SEQ ID NO: 2). In one embodiment, the NorA peptide inhibitor of the present disclosure comprises an amino acid sequence having 85%, 86%, 87%, 88%, 89%, 90%, 91%, 92%, 93%, 94%, 95%, 96%, 97%, 98%, or 99% sequence similarity to the sequence of SEQ ID NO 2.

In another embodiment, an exemplary NorA peptide inhibitor of the present disclosure comprises the amino acid sequence of YYYYAWRVGGYWR (SEQ ID NO: 3). In one embodiment, the NorA peptide inhibitor of the present disclosure comprises an amino acid sequence having 85%, 86%, 87%, 88%, 89%, 90%, 91%, 92%, 93%, 94%, 95%, 96%, 97%, 98%, or 99% sequence similarity to the sequence of SEQ ID NO 3.

In another embodiment, an exemplary NorA peptide inhibitor of the present disclosure comprises the amino acid sequence of GYYPYWRMYGFY (SEQ ID NO: 4). In one embodiment, the NorA peptide inhibitor of the present disclosure comprises an amino acid sequence having 85%, 86%, 87%, 88%, 89%, 90%, 91%, 92%, 93%, 94%, 95%, 96%, 97%, 98%, or 99% sequence similarity to the sequence of SEQ ID NO 4.

In another embodiment, an exemplary NorA peptide inhibitor of the present disclosure comprises the amino acid sequence of WETGYYPYWRMYGFYWALDY (SEQ ID NO: 6). In one embodiment, the NorA peptide inhibitor of the present disclosure comprises an amino acid sequence having 85%, 86%, 87%, 88%, 89%, 90%, 91%, 92%, 93%, 94%, 95%, 96%, 97%, 98%, or 99% sequence similarity to the sequence of SEQ ID NO 6.

In another embodiment, an exemplary NorA peptide inhibitor of the present disclosure comprises the amino acid sequence of YSRYYYYAWRVGGYWGGLDY (SEQ ID NO: 7). In one embodiment, the NorA peptide inhibitor of the present disclosure comprises an amino acid sequence having 85%, 86%, 87%, 88%, 89%, 90%, 91%, 92%, 93%, 94%, 95%, 96%, 97%, 98%, or 99% sequence similarity to the sequence of SEQ ID NO 7.

In another embodiment, an exemplary NorA peptide inhibitor of the present disclosure comprises or the amino acid sequence of MYWWWYWISASGFSWYALDY (SEQ ID NO: 8). In one embodiment, the NorA peptide inhibitor of the present disclosure comprises an amino acid sequence having 85%, 86%, 87%, 88%, 89%, 90%, 91%, 92%, 93%, 94%, 95%, 96%, 97%, 98%, or 99% sequence similarity to the sequence of SEQ ID NO 8.

In another embodiment, an exemplary NorA peptide inhibitor of the present disclosure comprises or the amino acid sequence of SDYYYKWYYSWGWYSYYAIDY (SEQ ID NO: 9). In one embodiment, the NorA peptide inhibitor of the present disclosure comprises an amino acid sequence having 85%, 86%, 87%, 88%, 89%, 90%, 91%, 92%, 93%, 94%, 95%, 96%, 97%, 98%, or 99% sequence similarity to the sequence of SEQ ID NO 9.

In another embodiment, an exemplary NorA peptide inhibitor of the present disclosure comprises or the amino acid sequence of YRSYVYGYWYWYWYWAMDY (SEQ ID NO: 10). In one embodiment, the NorA peptide inhibitor of the present disclosure comprises an amino acid sequence having 85%, 86%, 87%, 88%, 89%, 90%, 91%, 92%, 93%, 94%, 95%, 96%, 97%, 98%, or 99% sequence similarity to the sequence of SEQ ID NO 10.

In another embodiment, an exemplary NorA peptide inhibitor of the present disclosure comprises or the amino acid sequence of SGPYRNFYFHYISMYTVALDY (SEQ ID NO: 11). In one embodiment, the NorA peptide inhibitor of the present disclosure comprises an amino acid sequence having 85%, 86%, 87%, 88%, 89%, 90%, 91%, 92%, 93%, 94%, 95%, 96%, 97%, 98%, or 99% sequence similarity to the sequence of SEQ ID NO 11.

Another aspect of the disclosure relates to a NorA peptide inhibitor that comprises the amino acid sequence of X₁X₂X₃X₄X₅WRX₈X₉X₁₀X₁₁X₁₂ (SEQ ID NO: 27) where X₁ is Y, I, or V; X₂ is Y, F, or W; X₃ is Y, F, I, L, M, V, or W; X₄ is Y or W; X₅ is A, L, S, or V; X_{g} is V, A, D, F, G, H, I, L, M, R, W, or Y; X₉ is G, F, P, S, or T; X₁₀ is G, A, F, I, L, N, Q, S, T, V, or Y; X₁₁ is Y, A, C, F, H, I, L, M, R, S, T, V, or W; and X₁₂ is W, F, L, or Y.

In one embodiment, the NorA peptide inhibitor comprising the amino acid sequence of YSRYYYYAWRVGGYWGGLDY (SEQ ID NO: 7) except that the Y residue at position 4 is substituted with I or V.

In one embodiment, the NorA peptide inhibitor comprising the amino acid sequence of YSRYYYYAWRVGGYWGGLDY (SEQ ID NO: 7) except that the Y residue at position 5 is F or W.

In one embodiment, the NorA peptide inhibitor comprising the amino acid sequence of YSRYYYYAWRVGGYWGGLDY (SEQ ID NO: 7) except that the Y residue at position 6 is F, I, L, M, V, or W.

In one embodiment, the NorA peptide inhibitor comprising the amino acid sequence of YSRYYYYAWRVGGYWGGLDY (SEQ ID NO: 7) except that the Y residue at position 7 is W.

In one embodiment, the NorA peptide inhibitor comprising the amino acid sequence of YSRYYYYAWRVGGYWGGLDY (SEQ ID NO: 7) except that the A residue at position 8 is L, S, or V.

In one embodiment, the NorA peptide inhibitor comprising the amino acid sequence of YSRYYYYAWRVGGYWGGLDY (SEQ ID NO: 7) except that the V residue at position 11 is A, D, F, G, H, I, L, M, R, W, or Y.

In one embodiment, the NorA peptide inhibitor comprising the amino acid sequence of YSRYYYYAWRVGGYWGGLDY (SEQ ID NO: 7) except that the G residue at position 12 is F, P, S, or T.

In one embodiment, the NorA peptide inhibitor comprising the amino acid sequence of YSRYYYYAWRVGGYWGGLDY (SEQ ID NO: 7) except that the G residue at position 13 is A, F, I, L, N, Q, S, T, V, or Y.

In one embodiment, the NorA peptide inhibitor comprising the amino acid sequence of YSRYYYYAWRVGGYWGGLDY (SEQ ID NO: 7) except that the Y residue at position 13 is A, C, F, H, I, L, M, R, S, T, V, or W.

In one embodiment, the NorA peptide inhibitor comprising the amino acid sequence of YSRYYYYAWRVGGYWGGLDY (SEQ ID NO: 7) except that the W residue at position 14 is F, L, or Y.

The NorA peptide inhibitor as disclosed herein comprises the amino acid sequence of X₁YYX₄X₅WRX₈X₉GX₁₁X₁₂ (SEQ ID NO:1), X₁X₂X₃X₄X₅WRX₈X₉X₁₀X₁₁X₁₂ (SEQ ID NO: 27), or a fragment of one of those sequences. In some embodiments, the NorA peptide inhibitor comprises 12 amino acids in length. In some embodiments, the NorA peptide inhibitor comprises more than 12 amino acid residues in length. In some embodiments, the NorA peptide inhibitor comprises between 12-20 amino acid residues in length. In some embodiments, the NorA peptide inhibitor comprises between 20-30, 30-40, 40-50, 50-60, 60-70, 70-80, 80-90, or 90-100 amino acid residues in length. In some embodiments, the NorA peptide inhibitor comprises between 100-150, 150-200, 200-250, or 250-300 amino acid residues in length. In some embodiments, the NorA peptide inhibitor comprises >300 amino acid residues.

Also contemplated are cyclic (or circular) NorA peptide inhibitors derived from the above-identified peptides. Cyclic peptides are polypeptide chains taking the form of cyclic ring structure. According to one approach, called head-to-tail cyclization, the ring structure can be formed by linking one end of the peptide and the other end with an amide bond or other chemically stable bonds, e.g., lactone, ether, thioether, or disulfide. In other approaches, the head or tail (of the linear peptide) is linked to a side-chain of an amino acid residue near the other end, or side-chain to side-chain is used to link two internal residues near the N- and C-terminal ends. The chemistry for head-to-tail, head/tail-to-side chain, and side-chain to side-chain cyclization is well known in the art. Usually, cyclic peptides lacking amino and carboxy termini display more potent biological activity compared to their linear counterparts by increasing the resistance to hydrolysis from exopeptidases. Cyclic peptides can also show resistance to endopeptidases, which typically stems from an increase in conformational rigidity relative to linear peptides (Horton et al., “Exploring Privileged Structures: The Combinatorial Synthesis of Cyclic Peptides,” J. Comput. Aided. Mol. Des. 16:415-430 (2002), which is hereby incorporated by reference in its entirety). The reduced flexibility of cyclic peptides can also decrease the entropy term of the Gibbs binding free energy, thereby allowing enhanced binding toward target molecules, in this case NorA.

Another aspect of the present disclosure is directed to a protein scaffold comprising the NorA peptide inhibitor as described herein. Protein scaffolds offer a low-cost alternative to classic antibody therapeutic strategies. In addition, protein scaffolds are smaller in size than conventional antibodies, often more thermally stable and soluble, yet maintain the binding and antigenic specificity of monoclonal antibodies.

In any embodiment, the protein scaffold comprising the NorA peptide inhibitor as described herein is an antibody mimetic. Suitable antibody mimetics according to the present disclosure include, without limitation, a fibronectin type III (FN3) domain scaffold (i.e., monobody). These monobodies, also known as adnectins and pronectins, are based on the 10th or 14th extracellular domain of human fibronectin III, which can adopt an Ig-like β-sandwich fold with two or three exposed loops without a central disulfide bridge (see e.g., Koide et al., “Teaching an Old Scaffold New Tricks: Monobodies Constructed Using Alternative Surfaces of the FN3 Scaffold.” J Mol. Biol. 415:393-405 (2012), which is hereby incorporated by reference in its entirety).

In another embodiment the protein scaffold comprising the NorA peptide inhibitor as described herein is an A-domain protein scaffold, such as an affibody (Z-domain of protein A) (see, e.g., Nygren, PA. “Alternative Binding Proteins: Affibody Binding Proteins Developed from a Small Three-Helix Bundle Scaffold,” FEBS J. 275:2668-2676 (2008), which is hereby incorporated by reference in its entirety).

In another embodiment the protein scaffold comprising the NorA peptide inhibitor as described herein an albumin-binding domain (ABD)-Derived Affinity Protein (ADAPT) (ABD of protein G) (see, e.g., Nilvebrant et al., “The albumin-binding domain as a scaffold for protein engineering,” Computational Structural Biotechnology Journal 6(7): e201303009 (2013), which is hereby incorporated by reference in its entirety).

In another embodiment the protein scaffold comprising the NorA peptide inhibitor as described herein is a designed ankyrin repeat protein (DARPin). DARPins are small, engineered single-domain proteins that can be selected to bind to any protein target (see, e.g., Stumpp et al., “DARPins: A New Generation of Protein Therapeutics,” Drug Discov. Today 13:695-701 (2008), which is hereby incorporated by reference in its entirety).

In another embodiment the protein scaffold comprising the NorA peptide inhibitor as described herein is an anticalin. Anticalins are derived from lipocalins (which are abundant in humans, insects and many other organisms) that naturally form binding sites for small ligands by means of four structurally variable loops found in the proteins (see, e.g., Skerra, A. “Alternative Binding Proteins: Anticalins Harnessing the Structural Plasticity of the Lipocalin Ligand Pocket to Engineer Novel Binding Activities,” FEBS J. 275:2677-2683 (2008), which is hereby incorporated by reference in its entirety).

Another suitable antibody mimetic for use according to the present disclosure is a fynomer and nanofitins. Fynomers are 7-kDa binding proteins derived from SH3 domains of the human Fyn tyrosine kinase (see, e.g., Grabulovski et al., “A Novel, Non-Immunogenic Fyn SH3-Derived Binding Protein with Tumor Vascular Targeting Properties,” J. Biol. Chem. 282:3196-3204 (2007), which is hereby incorporated by reference in its entirety). Nanofitins (formerly Affitins) are structurally derived from the DNA binding protein Sac7d found in Sulfolobus acidocaldarius, an archeabacterium (see, e.g., Krehenbrink, M. “Artificial Binding Proteins (Affitins) as Probes for Conformational Changes in Secretin PulD,” J. Mol. Biol. 383:1058-1068 (2008), which is hereby incorporated by reference in its entirety).

Other suitable non-IgG protein scaffolds suitable for comprising the NorA peptide inhibitor disclosed herein include avimers, which are created from multimerized low-density lipoprotein receptor class A (LDLR-A) (see, e.g., Silverman, J. “Multivalent Avimer Proteins Evolved by Exon Shuffling of a Family of Human Receptor Domains,” Nat. Biotechnol. 23:1556-1561 (2005), which is hereby incorporated by reference in its entirety), knottins which are constructed from cysteine-rich knottin peptides (see, e.g., Kolmar, H, and A. Skerra. “Alternative Binding Proteins Get Mature: Rivalling Antibodies,” FEBS J. 275:2667 (2008), which is hereby incorporated by reference in its entirety), and affilins, which are derived from one of two proteins gamma-β crystallin (a family of 20-kDa proteins found in the eye lenses of vertebrates, including humans) or ubiquitin (a highly conserved 76-kDa protein found in eukaryotes) (see, e.g., Ebersbach, H. “Affilin-Novel Binding Molecules Based On Human Gamma-β-Crystallin: An All Beta-Sheet Protein,” J. Mol. Biol. 372:172-185 (2007), which is hereby incorporated by reference in its entirety).

### NorA Antibody-Based Molecules

NorA antibody-based molecules include, without limitation full antibodies, epitope binding fragments of whole antibodies, and antibody derivatives. An epitope binding fragment of an antibody can be obtained through the actual fragmenting of a parental antibody (for example, a Fab or (Fab)₂ fragment). Alternatively, the epitope binding fragment is an amino acid sequence that comprises a portion of the amino acid sequence of such parental antibody. As used herein, a molecule is said to be a “derivative” of an antibody (or relevant portion thereof) if it is obtained through the actual chemical modification of a parent antibody or portion thereof, or if it comprises an amino acid sequence that is substantially similar to the amino acid sequence of such parental antibody or relevant portion thereof (for example, differing by less than 30%, less than 20%, less than 10%, or less than 5% from such parental molecule or such relevant portion thereof, or by 10 amino acid residues, or by fewer than 10, 9, 8, 7, 6, 5, 4, 3 or 2 amino acid residues from such parental molecule or relevant portion thereof).

In an embodiment, a NorA antibody-based molecule of the present invention is an intact immunoglobulin or a molecule having an epitope-binding fragment thereof. As used herein, the terms “fragment”, “region”, “portion”, and “domain” are generally intended to be synonymous, unless the context of their use indicates otherwise. Naturally occurring antibodies typically comprise a tetramer, which is usually composed of at least two heavy (H) chains and at least two light (L) chains. Each heavy chain is comprised of a heavy chain variable (V_{H}) region and a heavy chain constant (C_{H}) region, usually comprised of three domains (C_{H}1, C_{H}2 and C_{H}3 domains). Heavy chains can be of any isotype, including IgG (IgG1, IgG2, IgG3 and IgG4 subtypes), IgA (IgA1 and IgA2 subtypes), IgM and IgE. Each light chain is comprised of a light chain variable (V_{L}) region and a light chain constant (C_{L}) region. Light chains include kappa chains and lambda chains. The heavy and light chain variable regions are typically responsible for antigen recognition, while the heavy and light chain constant regions may mediate the binding of the immunoglobulin to host tissues or factors, including various cells of the immune system (e.g., effector cells) and the first component (C1q) of the classical complement system. The V_{H} and V_{L} regions can be further subdivided into regions of hypervariability, termed “complementarity determining regions,” or “CDRs,” that are interspersed with regions of more conserved sequence, termed “framework regions” (FR). Each V_{H} and V_{L} region is composed of three CDR domains and four FR domains arranged from amino-terminus to carboxy-terminus in the following order: FR1-CDR1-FR2-CDR2-FR3-CDR3-FR4. The variable regions of the heavy and light chains contain a binding domain that interacts with an antigen. Of particular relevance are antibodies and their epitope-binding fragments that have been “isolated” so as to exist in a physical milieu distinct from that in which it may occur in nature or that have been modified so as to differ from a naturally-occurring antibody in amino acid sequence.

Fragments of antibodies (including Fab and (Fab)₂ fragments) that exhibit epitope-binding ability can be obtained, for example, by protease cleavage of intact antibodies. Single domain antibody fragments possess only one variable domain (e.g., V_{L} or V_{H}). Examples of the epitope-binding fragments encompassed within the present invention include (i) Fab′ or Fab fragments, which are monovalent fragments containing the V_{L}, V_{H}, C_{L} and C_{H}1 domains; (ii) F(ab′)₂ fragments, which are bivalent fragments comprising two Fab fragments linked by a disulfide bridge at the hinge region; (iii) Fd fragments consisting essentially of the V_{H} and C_{H}i domains; (iv) Fv fragments consisting essentially of a V_{L} and V_{H} domain, (v) dAb fragments (Ward et al. “Binding Activities Of A Repertoire Of Single Immunoglobulin Variable Domains Secreted From Escherichia coli,” Nature 341:544-546 (1989), which is hereby incorporated by reference in its entirety), which consist essentially of a V_{H} or V_{L} domain and also called domain antibodies (Holt et al. “Domain Antibodies: Proteins For Therapy,” Trends Biotechnol. 21(11):484-490 (2003), which is hereby incorporated by reference in its entirety); (vi) nanobodies (Revets et al. “Nanobodies As Novel Agents For Cancer Therapy,” Expert Opin. Biol. Ther. 5(1):111-124 (2005), which is hereby incorporated by reference in its entirety), and (vii) isolated complementarity determining regions (CDR). An epitope-binding fragment may contain 1, 2, 3, 4, 5 or all 6 of the CDR domains of such antibody. In an embodiment, a fragment (or region or portion or domain) of an antibody comprises, essentially consists of, or consists of 30 to 100 amino acids or 50 to 150 amino acids or 70 to 200 amino acids. In an embodiment, the length of a fragment (or region or portion or domain) of an antibody is at least 40%, 50%, 60%, 70%, 80%, 90% or 95% of the length of the antibody (full length antibody). In an embodiment, a fragment is an epitope binding fragment or a functional fragment of said antibody meaning it is expected it will elicit an activity of the antibody at least to some extent. “At least to some extent” may mean at least 20%, 30%, 40%, 50%, 60%, 70%, 80%, 90%, 95%, 100%, 150%, 200% or more.

Such antibody fragments may be obtained using conventional techniques known to those of skill in the art. For example, F(ab′)₂ fragments may be generated by treating a full-length antibody with pepsin. The resulting F(ab′)₂ fragment may be treated to reduce disulfide bridges to produce Fab′ fragments. Fab fragments may be obtained by treating an IgG antibody with papain and Fab′ fragments may be obtained with pepsin digestion of IgG antibody. A Fab′fragment may be obtained by treating an F(ab′)₂ fragment with a reducing agent, such as dithiothreitol. Antibody fragments may also be generated by expression of nucleic acids encoding such fragments in recombinant cells (see e.g., Evans et al. “Rapid Expression Of An Anti-Human C5 Chimeric Fab Utilizing A Vector That Replicates In COS And 293 Cells,” J. Immunol. Meth. 184:123-38 (1995), which is hereby incorporated by reference in its entirety). For example, a chimeric gene encoding a portion of a F(ab′)₂ fragment could include DNA sequences encoding the CH1 domain and hinge region of the heavy chain, followed by a translational stop codon to yield such a truncated antibody fragment molecule. Suitable fragments capable of binding to a desired epitope may be readily screened for utility in the same manner as an intact antibody.

Antibody derivatives include those molecules that contain at least one epitope-binding domain of an antibody, and are typically formed using recombinant techniques. One exemplary antibody derivative includes a single chain Fv (scFv). A scFv is formed from the two domains of the Fv fragment, the V_{L} region and the V_{H} region, which may be encoded by separate genes. Such gene sequences or their encoding cDNA are joined, using recombinant methods, by a flexible linker (typically of about 10, 12, 15 or more amino acid residues) that enables them to be made as a single protein chain in which the V_{L} and V_{H} regions associate to form monovalent epitope-binding molecules (see e.g., Bird et al. “Single-Chain Antigen-Binding Proteins,” Science 242:423-426 (1988); and Huston et al. “Protein Engineering Of Antibody Binding Sites: Recovery Of Specific Activity In An Anti-Digoxin Single-Chain Fv Analogue Produced In Escherichia coli,” Proc. Natl. Acad. Sci. (U.S.A.) 85:5879-5883 (1988), which are hereby incorporated by reference in their entirety). Alternatively, by employing a flexible linker that is not too short (e.g., not less than about 9 residues) to enable the V_{L} and V_{H} regions of a different single polypeptide chains to associate together, one can form a bispecific antibody, having binding specificity for two different epitopes.

In another embodiment, the antibody derivative is a divalent or bivalent single-chain variable fragment, engineered by linking two scFvs together either in tandem (i.e., tandem scFv), or such that they dimerize to form a diabody (Holliger et al. “‘Diabodies’: Small Bivalent And Bispecific Antibody Fragments,” Proc. Natl. Acad. Sci. (U.S.A.) 90(14), 6444-8 (1993), which is hereby incorporated by reference in its entirety). In yet another embodiment, the antibody is a triabody, i.e., a trivalent single chain variable fragment, engineered by linking three scFvs together, either in tandem or in a trimer formation to form a triabody. In another embodiment, the antibody is a tetrabody of four single chain variable fragments. In another embodiment, the antibody is a “linear antibody” which is an antibody comprising a pair of tandem Fd segments (V_{H}-C_{H}1-V_{H}-C_{H}1) that form a pair of antigen binding regions (see Zapata et al. Protein Eng. 8(10):1057-1062 (1995), which is hereby incorporated by reference in its entirety). In another embodiment, the antibody derivative is a minibody, consisting of the single-chain Fv regions coupled to the C_{H}3 region (i.e., scFv-C_{H}3).

These and other useful antibody fragments and derivatives in the context of the present invention are discussed further herein. It also should be understood that the term antibody-based molecule, unless specified otherwise, also includes antibody-like polypeptides, such as chimeric antibodies and humanized antibodies, and antibody fragments retaining the ability to specifically bind to the antigen (epitope-binding fragments or functional fragment) provided by any known technique, such as enzymatic cleavage, peptide synthesis, and recombinant techniques. In an embodiment, the wording “antibody-based molecule” may be replaced by the word “antibody” or by the expression “antibody or a functional fragment thereof”.

An antibody as generated herein may be of any isotype. As used herein, “isotype” refers to the immunoglobulin class (for instance IgG1, IgG2, IgG3, IgG4, IgD, IgA, IgE, or IgM) that is encoded by heavy chain constant region genes. The choice of isotype typically will be guided by the desired effector functions, such as antibody-dependent cellular cytotoxicity (ADCC) induction. Exemplary isotypes are IgG1, IgG2, IgG3, and IgG4. Particularly useful isotypes of the MuSK antibodies disclosed herein include IgG1 and IgG2.

In an embodiment, the antibody-based molecules of the present invention are “humanized,” particularly if they are to be employed for therapeutic purposes. The term “humanized” refers to a chimeric molecule, generally prepared using recombinant techniques, having an antigen-binding site derived from an immunoglobulin from a non-human species and a remaining immunoglobulin structure based upon the structure and/or sequence of a human immunoglobulin. The antigen-binding site may comprise either complete non-human antibody variable domains fused to human constant domains, or only the complementarity determining regions (CDRs) of such variable domains grafted to appropriate human framework regions of human variable domains. The framework residues of such humanized molecules may be wild-type (e.g., fully human) or they may be modified to contain one or more amino acid substitutions not found in the human antibody whose sequence has served as the basis for humanization. Humanization lessens or eliminates the likelihood that a constant region of the molecule will act as an immunogen in human individuals, but the possibility of an immune response to the foreign variable region remains (LoBuglio, A.F. et al. “Mouse/Human Chimeric Monoclonal Antibody In Man: Kinetics And Immune Response,” Proc. Natl. Acad. Sci. USA 86:4220-4224 (1989), which is hereby incorporated by reference in its entirety). Another approach focuses not only on providing human-derived constant regions, but modifying the variable regions so as to reshape them as closely as possible to human form. The variable regions of both heavy and light chains contain three complementarity-determining regions (CDRs) which vary in response to the antigens in question and determine binding capability. The CDRs are flanked by four framework regions (FRs) which are relatively conserved in a given species and which putatively provide a scaffolding for the CDRs. When non-human antibodies are prepared with respect to a particular antigen, the variable regions can be “reshaped” or “humanized” by grafting CDRs derived from non-human antibody onto the FRs present in the human antibody to be modified. Suitable methods for humanizing the non-human antibody described herein are known in the art see e.g., Sato, K. et al., Cancer Res 53:851-856 (1993); Riechmann, L. et al., “Reshaping Human Antibodies for Therapy,” Nature 332:323-327 (1988); Verhoeyen, M. et al., “Reshaping Human Antibodies: Grafting An Antilysozyme Activity,” Science 239:1534-1536 (1988); Kettleborough, C. A. et al., “Humanization Of A Mouse Monoclonal Antibody By CDR-Grafting: The Importance Of Framework Residues On Loop Conformation,” Protein Engineering 4:773-3783 (1991); Maeda, H. et al., “Construction Of Reshaped Human Antibodies With HIV-Neutralizing Activity,” Human Antibodies Hybridoma 2:124-134 (1991); Gorman, S. D. et al., “Reshaping A Therapeutic CD4 Antibody,” Proc. Natl. Acad. Sci. USA 88:4181-4185 (1991); Tempest, P. R. et al., “Reshaping A Human Monoclonal Antibody To Inhibit Human Respiratory Syncytial Virus Infection In Vivo,” Bio/Technology 9:266-271 (1991); Co, M. S. et al., “Humanized Antibodies For Antiviral Therapy,” Proc. Natl. Acad. Sci. USA 88:2869-2873 (1991); Carter, P. et al., “Humanization Of An Anti-p185her2 Antibody For Human Cancer Therapy,” Proc. Natl. Acad. Sci. USA 89:4285-4289 (1992); and Co, M. S. et al., “Chimeric And Humanized Antibodies With Specificity For The CD33 Antigen,” J. Immunol. 148:1149-1154 (1992), which are hereby incorporated by reference in their entirety. In some embodiments, humanized MuSK antibodies of the present invention preserve all CDR sequences (for example, a humanized antibody containing all six CDRs from the llama or mouse antibody). In other embodiments, humanized MuSK antibodies of the present invention have one or more CDRs (one, two, three, four, five, six) which are altered with respect to the original antibody. Methods of humanizing an antibody are well-known in the art and suitable for humanizing the antibodies disclosed herein (see, e.g., U.S. Pat. No. 5,225,539 to Winter; U.S. Pat. Nos. 5,530,101 and 5,585,089 to Queen and Selick; U.S. Pat. No. 5,859,205 to Robert et al.; U.S. Pat. No. 6,407,213 to Carter; and U.S. Pat. No. 6,881,557 to Foote, which are hereby incorporated by reference in their entirety).

In some antibodies only part of a CDR, namely the subset of CDR residues required for binding termed the “specificity determining residues” (“SDRs”), are needed to retain binding of the antibody. CDR residues not contacting antigen and not in the SDRs can be identified based on previous studies from regions of Kabat CDRs lying outside Chothia hypervariable loops (see, Kabat et al., SEQUENCES OF PROTEINS OF IMMUNOLOGICAL INTEREST, National Institutes of Health Publication No. 91-3242 (1992); Chothia, C. et al., “Canonical Structures For The Hypervariable Regions Of Immunoglobulins,” J. Mol. Biol. 196:901-917 (1987), which are hereby incorporated by reference in their entirety), by molecular modeling and/or empirically, or as described in Gonzales, N. R. et al., “SDR Grafting Of A Murine Antibody Using Multiple Human Germline Templates To Minimize Its Immunogenicity,” Mol. Immunol. 41:863-872 (2004), which is hereby incorporated by reference in its entirety. In such humanized antibodies, at positions in which one or more donor CDR residues is absent or in which an entire donor CDR is omitted, the amino acid residue occupying the position can be an amino acid residue occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of non-human amino acids in a humanized antibody and consequently decreasing potential immunogenicity. However, substitutions can also cause changes of affinity, and significant reductions in affinity are preferably avoided. Substitutions may also cause changes of activity. Such substitutions causing a significant reduction in activity are also preferably avoided. In this context, the antibody or antibody fragment should still exhibit a detectable activity of the antibody as earlier defined herein or an activity of the antibody at least to some extent. Positions for substitution within CDRs and amino acids to substitute can also be selected empirically.

Phage display technology can alternatively be used to increase (or decrease) CDR affinity of the antibody-based molecules of the present invention. This technology, referred to as affinity maturation, employs mutagenesis or “CDR walking” and re-selection using the target antigen or an antigenic fragment thereof to identify antibodies having CDRs that bind with higher (or lower) affinity to the antigen when compared with the initial or parental antibody (see, e.g. Glaser et al., “Antibody Engineering By Codon-Based Mutagenesis In A Filamentous Phage Vector System,” J. Immunology 149:3903-3913 (1992), which is hereby incorporated by reference in its entirety). Mutagenizing entire codons rather than single nucleotides results in a semi-randomized repertoire of amino acid mutations. Libraries can be constructed consisting of a pool of variant clones each of which differs by a single amino acid alteration in a single CDR from another member of such library and which contain variants potentially representing each possible amino acid substitution for each CDR residue. Mutants with increased (or decreased) binding affinity for the antigen can be screened by contacting the immobilized mutants with labeled antigen. Any screening method known in the art can be used to identify variant antibody-based binding molecules with increased or decreased affinity to the antigen (e.g., ELISA) (See Wu, H. et al., “Stepwise In Vitro Affinity Maturation Of Vitaxin, An Alphav Beta3-Specific Humanized mAb,” Proc. Natl. Acad. Sci. USA 95:6037-6042 (1998); Yelton et al., “Affinity Maturation Of The BR96 Anti-Carcinoma Antibody By Codon-Based Mutagenesis,” J. Immunology 155:1994 (1995), which are hereby incorporated by reference in their entirety). CDR walking, which randomizes the light chain may be used (see, Schier, R. et al., “Isolation Of Picomolar Affinity Anti-c-erbB-2 Single-Chain Fv By Molecular Evolution Of The Complementarity Determining Regions In The Center Of The Antibody Binding Site,” J. Mol. Biol. 263:551-567 (1996), which is hereby incorporated by reference in its entirety).

In an aspect of the present invention, the NorA-antibody based molecule as described herein comprises the amino acid sequence of any one, any two, any three, any four, any five, or any six CDRs as provided in Tables 1 and 2 herein.

In one aspect, the antibody-based molecule that binds NorA comprises a heavy chain variable region, wherein said heavy chain variable region comprises: a complementarity-determining region 1 (CDR-H1) comprising an amino acid sequence of any one of SEQ ID NOs: 12-14, or a modified amino acid sequence of any one of SEQ ID NOs: 12-14, said modified sequence having at least 80% sequence identity to any one of SEQ ID NOs: 12-14; a complementarity-determining region 2 (CDR-H2) comprising an amino acid sequence of any one of SEQ ID NOs: 15-20, or a modified amino acid sequence of any one of SEQ ID NOs: 15-20, said modified sequences having at least 80% sequence identity to any one of SEQ ID NOs: 15-20; and/or a complementarity-determining region 3 (CDR-H3) comprising an amino acid sequence of any one of SEQ ID NOs: 5-11, or a modified amino acid sequence of any one of SEQ ID NO: 5-11, said modified sequence having at least 80% sequence identity to any one of SEQ ID NOs: 5-11.

In any embodiment, the antibody-based molecule that binds NorA comprises a heavy chain variable region selected from:
(i) a heavy chain variable region comprising the CDR-H1 of SEQ ID NO: 12, the CDR-H2 of SEQ ID NO: 15, and the CDR-H3 of SEQ ID NO: 5 (N3-24);
(ii) a heavy chain variable region comprising the CDR-H1 of SEQ ID NO: 13, the CDR-H2 of SEQ ID NO: 16, and the CDR-H3 of SEQ ID NO: 6 (N3-25);
(iii) a heavy chain variable region comprising the CDR-H1 of SEQ ID NO: 13, the CDR-H2 of SEQ ID NO: 16, and the CDR-H3 of SEQ ID NO: 7 (N3-36);
(iv) a heavy chain variable region comprising the CDR-H1 of SEQ ID NO: 13, the CDR-H2 of SEQ ID NO: 17, and the CDR-H3 of SEQ ID NO: 8 (N3-38);
(v) a heavy chain variable region comprising the CDR-H1 of SEQ ID NO: 13, the CDR-H2 of SEQ ID NO: 18, and the CDR-H3 of SEQ ID NO: 9 (N3-39);
(vi) a heavy chain variable region comprising the CDR-H1 of SEQ ID NO: 13, the CDR-H2 of SEQ ID NO: 19, and the CDR-H3 of SEQ ID NO: 10 (N3-41); and
(vii) a heavy chain variable region comprising the CDR-H1 of SEQ ID NO: 14, the CDR-H2 of SEQ ID NO: 20, and the CDR-H3 of SEQ ID NO: 11 (N3-45).

The sequences of the heavy chain CDR sequences are provided in Table 1 below.

TABLE 1
Heavy Chain CDR Sequences of
NorA Antibodies

| Ab | CDR1- | SEQ | CDR2- | SEQ | CDR3- | SEQ ID |
|---|---|---|---|---|---|---|
| Clone | HC | ID NO: | HC | ID NO: | HC | NO: |
| N3-24 | ISSSSIH | 12 | SIYSY | 15 | QSWHY | 5 |
| | | | SGSTS | | TYFRY | |
| | | | YADSV | | SSYYY | |
| | | | KG | | QGALD | |
| | | | | | Y | |
| N3-25 | SSSIH | 13 | SISSS | 16 | WETGY | 6 |
| | | | SGSTS | | YPYWR | |
| | | | YADSV | | MYGFY | |
| | | | KG | | WALDY | |
| N3-36 | SSSIH | 13 | SISSS | 16 | YSRYY | 7 |
| | | | SGSTS | | YYAWR | |
| | | | YADSV | | VGGYW | |
| | | | KG | | GGLDY | |
| N3-38 | SSSIH | 13 | SISSS | 17 | MYWWW | 8 |
| | | | YGSTS | | YWISA | |
| | | | YADSV | | SGFSW | |
| | | | KG | | YALDY | |
| N3-39 | SSSIH | 13 | SISPY | 18 | SDYYY | 9 |
| | | | SGYTS | | KWYYS | |
| | | | YADSV | | WGWYS | |
| | | | KG | | YYAID | |
| | | | | | Y | |
| N3-41 | SSSIH | 13 | SISSS | 19 | YRSYV | 10 |
| | | | SGSTS | | YGYWY | |
| | | | YADSV | | WYWYW | |
| | | | KG | | AMDY | |
| N3-45 | SYSIH | 14 | SIYSS | 20 | SGPYR | 11 |
| | | | SGSTY | | NFYFH | |
| | | | YADSV | | YISMY | |
| | | | KG | | TVALD | |
| | | | | | Y | |

In any embodiment, the NorA antibody-based molecule as disclosed herein is a single-domain antibody (nanobody).

In any embodiment, the NorA antibody-based molecule, particularly a single-domain antibody as disclosed herein comprises human immunoglobulin heavy chain framework regions.

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, or at least 99% identical to SEQ ID NO: 28. In any embodiment, the NorA antibody-based molecule as disclosed herein comprises a heavy chain variable region comprising an amino acid sequence of SEQ ID NO: 28.

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, or at least 99% identical to SEQ ID NO: 30. In any embodiment, the NorA antibody-based molecule as disclosed herein comprises a heavy chain variable region comprising an amino acid sequence of SEQ ID NO: 30.

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, or at least 99% identical to SEQ ID NO: 32. In any embodiment, the NorA antibody-based molecule as disclosed herein comprises a heavy chain variable region comprising an amino acid sequence of SEQ ID NO: 32.

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, or at least 99% identical to SEQ ID NO: 34. In any embodiment, the NorA antibody-based molecule as disclosed herein comprises a heavy chain variable region comprising an amino acid sequence of SEQ ID NO: 34.

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, or at least 99% identical to SEQ ID NO: 36. In any embodiment, the NorA antibody-based molecule as disclosed herein comprises a heavy chain variable region comprising an amino acid sequence of SEQ ID NO: 36.

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, or at least 99% identical to SEQ ID NO: 38. In any embodiment, the NorA antibody-based molecule as disclosed herein comprises a heavy chain variable region comprising an amino acid sequence of SEQ ID NO: 38.

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, or at least 99% identical to SEQ ID NO: 40. In any embodiment, the NorA antibody-based molecule as disclosed herein comprises a heavy chain variable region comprising an amino acid sequence of SEQ ID NO: 40.

In any embodiment, the NorA antibody-based molecule disclosed herein further comprises a light chain variable region. The light chain variable region of the NorA antibody-based molecule comprises: a complementarity-determining region 1 (CDR-L1) having an amino acid sequence of SEQ ID NOs: 21, or a modified amino acid sequence of SEQ ID NO: 21, said modified sequence having at least 80% sequence identity to SEQ ID NO: 21; a complementarity-determining region 2 (CDR-L2) having an amino acid sequence of SEQ ID NO: 22, or a modified amino acid sequence of SEQ ID NO: 22, said modified sequence having at least 80% sequence identity to SEQ ID NO: 22; and a complementarity-determining region 3 (CDR-L3) having an amino acid sequence of any one of SEQ ID NOs: 23-26, or a modified amino acid sequence of any one of SEQ ID NO: 23-26, said modified sequence having at least 80% sequence identity to any one of SEQ ID NO: 23-26.

In any embodiment, the NorA antibody-based molecule as disclosed herein comprises a light chain variable region is selected from:
(i) a light chain variable region comprising the CDR-L1 of SEQ ID NO: 21, the CDR-L2 of SEQ ID NO: 22, and the CDR-L3 of SEQ ID NO: 23;
(ii) a light chain variable region comprising the CDR-L1 of SEQ ID NO: 21, the CDR-L2 of SEQ ID NO: 22, and the CDR-L3 of SEQ ID NO: 24;
(iv) a light chain variable region comprising the CDR-L1 of SEQ ID NO: 21, the CDR-L2 of SEQ ID NO: 22, and the CDR-L3 of SEQ ID NO: 25; and
(v) a light chain variable region comprising the CDR-L1 of SEQ ID NO: 21, the CDR-L2 of SEQ ID NO: 22, and the CDR-L3 of SEQ ID NO: 26.

The sequences of the light chain CDR sequences are provided in Table 2 below.

TABLE 2
Light Chain CDR Sequences of
NorA Antibodies

| Antibody | CDR1- | SEQ ID | CDR2- | SEQ ID | CDR3- | SEQ ID |
|---|---|---|---|---|---|---|
| Clone | LC | NO: | LC | NO: | LC | NO: |
| N3-24 | RASQS | 21 | SASSL | 22 | QQSYY | 23 |
| | VSSAV | | YS | | GHLIT | |
| | A | | | | | |
| N3-25 | RASQS | 21 | SASSL | 22 | QQSSS | 24 |
| | VSSAV | | YS | | SLIT | |
| | A | | | | | |
| N3-36 | RASQS | 21 | SASSL | 22 | QQSSS | 24 |
| | VSSAV | | YS | | SLIT | |
| | A | | | | | |
| N3-38 | RASQS | 21 | SASSL | 22 | QQSYY | 25 |
| | VSSAV | | YS | | PKPFT | |
| | A | | | | | |
| N3-39 | RASQS | 21 | SASSL | 22 | QQSSS | 24 |
| | VSSAV | | YS | | SLIT | |
| | A | | | | | |
| N3-41 | RASQS | 21 | SASSL | 22 | QQSSS | 24 |
| | VSSAV | | YS | | SLIT | |
| | A | | | | | |
| N3-45 | RASQS | 21 | SASSL | 22 | QQYYF | 26 |
| | VSSAV | | YS | | YPLIT | |
| | A | | | | | |

In any embodiment, the light chain variable region of the antibody-based molecule further includes human immunoglobulin light chain framework regions.

In any embodiment, the NorA antibody-based molecule as disclosed herein comprises a light chain variable region comprising an amino acid sequence that is at least 80%, at least 85%, at least 90%, at least 95%, at least 96%, at least 97%, at least 98%, or at least 99% identical to SEQ ID NO: 29. In any embodiment, the NorA antibody-based molecule as disclosed herein comprises a light chain variable region comprising an amino acid sequence of SEQ ID NO: 29.

In any embodiment, the NorA antibody-based molecule as disclosed herein comprises a light chain variable region comprising an amino acid sequence that is at least 80%, at least 85%, at least 90%, at least 95%, at least 96%, at least 97%, at least 98%, or at least 99% identical to SEQ ID NO: 31. In any embodiment, the NorA antibody-based molecule as disclosed herein comprises a light chain variable region comprising an amino acid sequence of SEQ ID NO: 31.

In any embodiment, the NorA antibody-based molecule as disclosed herein comprises a light chain variable region comprising an amino acid sequence that is at least 80%, at least 85%, at least 90%, at least 95%, at least 96%, at least 97%, at least 98%, or at least 99% identical to SEQ ID NO: 33. In any embodiment, the NorA antibody-based molecule as disclosed herein comprises a light chain variable region comprising an amino acid sequence of SEQ ID NO: 33.

In any embodiment, the NorA antibody-based molecule as disclosed herein comprises a light chain variable region comprising an amino acid sequence that is at least 80%, at least 85%, at least 90%, at least 95%, at least 96%, at least 97%, at least 98%, or at least 99% identical to SEQ ID NO: 35. In any embodiment, the NorA antibody-based molecule as disclosed herein comprises a light chain variable region comprising an amino acid sequence of SEQ ID NO: 35.

In any embodiment, the NorA antibody-based molecule as disclosed herein comprises a light chain variable region comprising an amino acid sequence that is at least 80%, at least 85%, at least 90%, at least 95%, at least 96%, at least 97%, at least 98%, or at least 99% identical to SEQ ID NO: 37. In any embodiment, the NorA antibody-based molecule as disclosed herein comprises a light chain variable region comprising an amino acid sequence of SEQ ID NO: 37.

In any embodiment, the NorA antibody-based molecule as disclosed herein comprises a light chain variable region comprising an amino acid sequence that is at least 80%, at least 85%, at least 90%, at least 95%, at least 96%, at least 97%, at least 98%, or at least 99% identical to SEQ ID NO: 39. In any embodiment, the NorA antibody-based molecule as disclosed herein comprises a light chain variable region comprising an amino acid sequence of SEQ ID NO: 39.

In any embodiment, the NorA antibody-based molecule as disclosed herein comprises a light chain variable region comprising an amino acid sequence that is at least 80%, at least 85%, at least 90%, at least 95%, at least 96%, at least 97%, at least 98%, or at least 99% identical to SEQ ID NO: 41. In any embodiment, the NorA antibody-based molecule as disclosed herein comprises a light chain variable region comprising an amino acid sequence of SEQ ID NO: 41.

In any embodiment, the NorA antibody-based molecule as disclosed herein comprises a heavy chain variable region comprising the CDR-H1 of SEQ ID NO: 12, the CDR-H2 of SEQ ID NO: 15, and the CDR-H3 of SEQ ID NO: 5, and a light chain variable region comprising the CDR-L1 of SEQ ID NO: 21, the CDR-L2 of SEQ ID NO: 22, and the CDR-L3 of SEQ ID NO: 23 (N3-24).

In any embodiment, the NorA antibody-based molecule as disclosed herein comprises a heavy chain variable region comprising the CDR-H1 of SEQ ID NO: 13, the CDR-H2 of SEQ ID NO: 16, and the CDR-H3 of SEQ ID NO: 6, and a light chain variable region comprising the CDR-L1 of SEQ ID NO: 21, the CDR-L2 of SEQ ID NO: 22, and the CDR-L3 of SEQ ID NO: 24 (N3-25).

In any embodiment, the NorA antibody-based molecule as disclosed herein comprises a heavy chain variable region comprising the CDR-H1 of SEQ ID NO: 13, the CDR-H2 of SEQ ID NO: 16, and the CDR-H3 of SEQ ID NO: 7, and a light chain variable region comprising the CDR-L1 of SEQ ID NO: 21, the CDR-L2 of SEQ ID NO: 22, and the CDR-L3 of SEQ ID NO: 24 (N3-36).

In any embodiment, the NorA antibody-based molecule as disclosed herein comprises a heavy chain variable region comprising the CDR-H1 of SEQ ID NO: 13, the CDR-H2 of SEQ ID NO: 17, and the CDR-H3 of SEQ ID NO: 8, and a light chain variable region comprising the CDR-L1 of SEQ ID NO: 21, the CDR-L2 of SEQ ID NO: 22, and the CDR-L3 of SEQ ID NO: 25 (N3-38).

In any embodiment, the NorA antibody-based molecule as disclosed herein comprises a heavy chain variable region comprising the CDR-H1 of SEQ ID NO: 13, the CDR-H2 of SEQ ID NO: 18, and the CDR-H3 of SEQ ID NO: 9, and a light chain variable region comprising the CDR-L1 of SEQ ID NO: 21, the CDR-L2 of SEQ ID NO: 22, and the CDR-L3 of SEQ ID NO: 24 (N3-39).

In any embodiment, the NorA antibody-based molecule as disclosed herein comprises a heavy chain variable region comprising the CDR-H1 of SEQ ID NO: 13, the CDR-H2 of SEQ ID NO: 19, and the CDR-H3 of SEQ ID NO: 10, and a light chain variable region comprising the CDR-L1 of SEQ ID NO: 21, the CDR-L2 of SEQ ID NO: 22, and the CDR-L3 of SEQ ID NO: 24 (N3-41).

In any embodiment, the NorA antibody-based molecule as disclosed herein comprises a heavy chain variable region comprising the CDR-H1 of SEQ ID NO: 14, the CDR-H2 of SEQ ID NO: 20, and the CDR-H3 of SEQ ID NO: 11, and a light chain variable region comprising the CDR-L1 of SEQ ID NO: 21, the CDR-L2 of SEQ ID NO: 22, and the CDR-L3 of SEQ ID NO: 26 (N3-45).

The NorA antibody-based molecule as described herein may comprise a variable light (VL) chain, a variable heavy (VH) chain, or a combination of VL and VH chains. In some embodiments, the VH chain of the NorA antibody-based molecule comprises any one of the VH amino acid sequences provided in Table 3 below, or an amino acid sequence that is at least 60%, at least 65%, at least 70%, at least 75%, at least 80%, at least 85%, at least 90%, at least 95% identical to any one of the VH amino acid sequences listed in Table 3. In some embodiments, the VL chain of the NorA antibody-based molecule comprises any one of the VL amino acid sequences provided in Table 3 below, or an amino acid sequence that is at least 60%, at least 65%, at least 70%, at least 75%, at least 80%, at least 85%, at least 90%, at least 95% identical to any one of the VL amino acid sequences listed in Table 3.

TABLE 3
Heavy and Light Chain Sequences
of NorA Antibodies

| Antibody | | SEQ ID | |
|---|---|---|---|
| Clone | Chain | NO: | Sequence |
| N3-24 | V_{H} | 28 | EVQLVESGGGLVQPGGSLRLSCAAS |
| | | | GFTISSSSIHWVRQAPGKGLEWVAS |
| | | | IYSYSGSTSYADSVKGRFTISADTS |
| | | | KNTAYLQMNSLRAEDTAVYYCARQS |
| | | | WHYTYFRYSSYYYQGALDYWGQGTL |
| | | | VTVSS |
| | V_{L} | 29 | DIQMTQSPSSLSASVGDRVTITCRA |
| | | | SQSVSSAVAWYQQKPGKAPKLLIYS |
| | | | ASSLYSGVPSRFSGSRSGTDFTLTI |
| | | | SSLQPEDFATYYCQQSYYGHLITFG |
| | | | QGTKVEIKRTV |
| N3-25 | V_{H} | 30 | EVQLVESGGGLVQPGGSLRLSCAAS |
| | | | GFTFSSSSIHWVRQAPGKGLEWVAS |
| | | | ISSSSGSTSYADSVKGRFTISADTS |
| | | | KNTAYLQMNSLRAEDTAVYYCARWE |
| | | | TGYYPYWRMYGFYWALDYWGQGTLV |
| | | | TVSS |
| | V_{L} | 31 | DIQMTQSPSSLSASVGDRVTITCRA |
| | | | SQSVSSAVAWYQQKPGKAPKLLIYS |
| | | | ASSLYSGVPSRFSGSRSGTDFTLTI |
| | | | SSLQPEDFATYYCQQSSSSLITFGQ |
| | | | GTKVEIKRTV |
| N3-36 | V_{H} | 32 | EVQLVESGGGLVQPGGSLRLSCAAS |
| | | | GFTFSSSSIHWVRQAPGKGLEWVAS |
| | | | ISSSSGSTSYADSVKGRFTISADTS |
| | | | KNTAYLQMNSLRAEDTAVYYCARYS |
| | | | RYYYYAWRVGGYWGGLDYWGQGTLV |
| | | | TVSS |
| | V_{L} | 33 | DIQMTQSPSSLSASVGDRVTITCRA |
| | | | SQSVSSAVAWYQQKPGKAPKLLIYS |
| | | | ASSLYSGVPSRFSGSRSGTDFTLTI |
| | | | SSLQPEDFATYYCQQSSSSLITFGQ |
| | | | GTKVEIKRTV |
| N3-38 | V_{H} | 34 | EVQLVESGGGLVQPGGSLRLSCAAS |
| | | | GFTLYSSSIHWVRQAPGKGLEWVAS |
| | | | ISSSYGSTSYADSVKGRFTISADTS |
| | | | KNTAYLQMNSLRAEDTAVYYCARMY |
| | | | WWWYWISASGFSWYALDYWGQGTLV |
| | | | TVSS |
| | V_{L} | 35 | DIQMTQSPSSLSASVGDRVTITCRA |
| | | | SQSVSSAVAWYQQKPGKAPKLLIYS |
| | | | ASSLYSGVPSRFSGSRSGTDFTLTI |
| | | | SSLQPEDFATYYCQQSYYPKPFTFG |
| | | | QGTKVEIKRTV |
| N3-39 | V_{H} | 36 | EVQLVESGGGLVQPGGSLRLSCAAS |
| | | | GFTFSSSSIHWVRQAPGKGLEWVAS |
| | | | ISPYSGYTSYADSVKGRFTISADTS |
| | | | KNTAYLQMNSLRAEDTAVYYCARSD |
| | | | YYYKWYYSWGWYSYYAIDYWGQGTL |
| | | | VTVSS |
| | V_{L} | 37 | DIQMTQSPSSLSASVGDRVTITCRA |
| | | | SQSVSSAVAWYQQKPGKAPKLLIYS |
| | | | ASSLYSGVPSRFSGSRSGTDFTLTI |
| | | | SSLQPEDFATYYCQQSSSSLITFGQ |
| | | | GTKVEIKRTV |
| N3-41 | V_{H} | 38 | EVQLVESGGGLVQPGGSLRLSCAAS |
| | | | GFTFSSSSIHWVRQAPGKGLEWVAS |
| | | | ISSSSGSTSYADSVKGRFTISADTS |
| | | | KNTAYLQMNSLRAEDTAVYYCARYR |
| | | | SYVYGYWYWYWYWAMDYWGQGTLVT |
| | | | VSS |
| | V_{L} | 39 | DIQMTQSPSSLSASVGDRVTITCRA |
| | | | SQSVSSAVAWYQQKPGKAPKLLIYS |
| | | | ASSLYSGVPSRFSGSRSGTDFTLTI |
| | | | SSLQPEDFATYYCQQSSSSLITFGQ |
| | | | GTKVEIKRTV |
| N3-45 | V_{H} | 40 | EVQLVESGGGLVQPGGSLRLSCAAS |
| | | | GFTISSYSIHWVRQAPGKGLEWVAS |
| | | | IYSSSGSTYYADSVKGRFTISADTS |
| | | | KNTAYLQMNSLRAEDTAVYYCARSG |
| | | | PYRNFYFHYISMYTVALDYWGQGTL |
| | | | VTVSS |
| | V_{L} | 41 | DIQMTQSPSSLSASVGDRVTITCRA |
| | | | SQSVSSAVAWYQQKPGKAPKLLIYS |
| | | | ASSLYSGVPSRFSGSRSGTDFTLTI |
| | | | SSLQPEDFATYYCQQYYFYPLITFG |
| | | | QGTKVEIKRTV |

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, at least 99%, or 100% identical to SEQ ID NO: 28; and a light chain variable region comprising an amino acid sequence that is at least 80%, at least 85%, at least 90%, at least 95%, at least 96%, at least 97% at least 98%, at least 99%, or 100% identical to SEQ ID NO: 29 (N3-24).

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, at least 99% or 100% identical to SEQ TD NO: 30; and a light chain variable region comprising an amino acid sequence that is at least 80%, at least 85%, at least 90%, at least 95%, at least 96%, at least 97%, at least 98%, at least 99%, or 100% identical to SEQ ID NO: 31 (N3-25).

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, at least 99%, or 100% identical to SEQ ID NO: 32; and a light chain variable region comprising an amino acid sequence that is at least 80%, at least 85%, at least 90%, at least 95%, at least 96%, at least 97%, at least 98%, at least 99%, or 100% identical to SEQ ID NO: 33 (N3-36).

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, at least 99%, or 100% identical to SEQ ID NO: 34; and a light chain variable region comprising an amino acid sequence that is at least 80%, at least 85%, at least 90%, at least 95%, at least 96%, at least 97%, at least 98%, at least 99%, or 100% identical to SEQ ID NO: 35 (N3-38).

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, at least 99%, or 100% identical to SEQ ID NO: 36; and a light chain variable region comprising an amino acid sequence that is at least 80%, at least 85%, at least 90%, at least 95%, at least 96%, at least 97%, at least 98%, at least 99%, or 100% identical to SEQ ID NO: 37 (N3-39).

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, at least 99%, or 100% identical to SEQ ID NO: 38; and a light chain variable region comprising an amino acid sequence that is at least 80%, at least 85%, at least 90%, at least 95%, at least 96%, at least 97%, at least 98%, at least 99%, or 100% identical to SEQ ID NO: 39 (N3-41).

In any embodiment, the NorA antibody-based molecule as disclosed herein comprises a heavy chain variable region comprising an amino acid sequence that is at least 80%, at least 85%, at least 90%, at least 95%, at least 96%, at least 97%, at least 98%, at least 99%, or 100% identical to SEQ ID NO: 40; and a light chain variable region comprising an amino acid sequence that is at least 80%, at least 85%, at least 90%, at least 95%, at least 96%, at least 97%, at least 98%, at least 99%, or 100% identical to SEQ ID NO: 41 (N3-45).

In any embodiment, the NorA antibody-based molecule as disclosed herein is a monoclonal antibody or binding fragment thereof.

In any embodiment, the NorA antibody-based molecule as disclosed herein is a full-length antibody, an epitope-binding fragment of an antibody, or an antibody derivative.

In any embodiment, the NorA antibody-based molecule as disclosed herein is an epitope binding fragment selected from a F(ab) fragment, a F(ab′) fragment, and F(ab′)₂ fragment.

In any embodiment, the NorA antibody-based molecule as disclosed herein is a F(ab) fragment.

In any embodiment, the NorA antibody-based molecule as disclosed herein is an antibody derivative selected from the group consisting of a scFv, a minibody, a diabody, a triabody, a tribody and a tetrabody.

Another aspect of the disclosure is directed to isolated polynucleotides encoding the NorA peptide inhibitors as described supra, isolated polynucleotides encoding the protein scaffolds comprising the NorA peptide inhibitor as disclosed supra, or isolated polynucleotides encoding the antibody-based molecule as disclosed supra. In any embodiment, the isolated polynucleotide is a messenger RNA (mRNA) molecule, a DNA molecule, or a hybrid molecule. In any embodiment, the isolated polynucleotide is a messenger RNA (mRNA) molecule.

The polynucleotides of the present disclosure may be produced by chemical synthesis such as solid phase polynucleotide synthesis on an automated polynucleotide synthesizer and assembled into complete single or double stranded molecules. Alternatively, the polynucleotides of the invention may be produced by other techniques such as PCR followed by routine cloning. Techniques for producing or obtaining polynucleotides of a given sequence are well known in the art.

The polynucleotides of the disclosure may comprise at least one non-coding sequence, such as a promoter or enhancer sequence, intron, polyadenylation signal, a cis sequence facilitating RepA binding, and the like. The polynucleotide sequences may also comprise additional sequences encoding for example a linker sequence, a marker or a tag sequence, such as a histidine tag or an HA tag to facilitate purification or detection of the protein, a signal sequence, a fusion protein partner such as RepA, Fc portion, or bacteriophage coat protein such as pIX or pIII.

Another embodiment of the disclosure is directed to a vector comprising at least one polynucleotide encoding the NorA peptide inhibitors as described supra, a vector comprising at least one polynucleotide encoding the protein scaffolds comprising the NorA peptide inhibitor as disclosed supra, or a vector comprising at least one polynucleotide encoding the antibody-based molecule as disclosed supra. Such vectors include, without limitation, plasmid vectors, viral vectors, including without limitation, vaccina vector, lentiviral vector, adenoviral vector, adeno-associated viral vector, vectors for baculovirus expression, transposon based vectors or any other vector suitable for introduction of the polynucleotides described herein into a given organism or genetic background by any means to facilitate expression of the encoded antibody polypeptide. In one embodiment, the polynucleotide sequence encoding the heavy chain variable domain, alone or together with the polynucleotide sequence encoding the light chain variable domain as described herein, are combined with sequences of a promoter, a translation initiation segment (e.g., a ribosomal binding sequence and start codon), a 3′ untranslated region, polyadenylation signal, a termination codon, and transcription termination to form one or more expression vector constructs.

In any embodiment, an expression vector construct encoding the NorA antibody-based molecule includes the polynucleotide sequence encoding the heavy chain polypeptide, a functional fragment thereof, a variant thereof, or combinations thereof. The expression construct can alternatively include a nucleic acid sequence encoding the light chain polypeptide, a functional fragment thereof, a variant thereof, or combinations thereof. In an embodiment, the expression vector construct includes a nucleic acid sequence encoding the heavy chain polypeptide, a functional fragment thereof, or a variant thereof, and the light chain polypeptide, a functional fragment thereof, or a variant thereof.

In an embodiment, the expression constructs encoding the NorA peptide inhibitor, NorA peptide inhibitor protein scaffolds, or NorA antibody-based molecule further comprise a promoter sequence suitable for driving expression of the NorA peptide inhibitor or NorA antibody-based molecule. Suitable promoter sequences include, without limitation, the elongation factor 1-alpha promoter (EF1a) promoter, a phosphoglycerate kinase-1 promoter (PGK) promoter, a cytomegalovirus immediate early gene promoter (CMV), a chimeric liver-specific promoter (LSP), a cytomegalovirus enhancer/chicken beta-actin promoter (CAG), a tetracycline responsive promoter (TRE), a transthyretin promoter (TTR), a simian virus 40 promoter (SV40) and a CK6 promoter. Other promoters suitable for driving gene expression in mammalian cells that are known in the art are also suitable for incorporation into the expression constructs disclosed herein.

In an embodiment, the expression construct further encodes a linker sequence. The linker sequence can encode an amino acid sequence that spatially separates and/or links the one or more components of the expression construct (heavy chain and light chain components of the encoded antibody).

Another aspect of the present disclosure is directed to a delivery vehicle comprising the isolated polynucleotides or the vectors disclosed herein that are suitable for administration to a subject in accordance with the methods disclosed herein. Suitable delivery vehicles include, without limitation, nanoparticle delivery vehicles, lipid-based particle delivery vehicles, or polymer-based particle delivery vehicles.

In some embodiments, the delivery vehicle is a lipid-based particle delivery vehicle. Suitable lipid-based vehicles include cationic lipid based lipoplexes (e.g., 1,2-dioleoyl-3trimethylammonium-propane (DOTAP)), neutral lipids based lipoplexes (e.g., cholesterol and dioleoylphosphatidyl ethanolamine (DOPE)), anionic lipid based lipoplexes (e.g., cholesteryl hemisuccinate (CHEMS)), and pH-sensitive lipid lipoplexes (e.g., 2,3-dioleyloxy-N-[2(sperminecarboxamido)ethyl]-N,N-dimethyl-1-propanaminium trifluoroacetate (DOSPA)). Other suitable lipid-based delivery particles incorporate ionizable DOSPA in lipofectamine and DLin-MC3-DMA ((6Z,9Z,28Z,31Z)-heptatriaconta-6,9,28,31-tetraen-19-yl-4-(dimethylamino) butanoate).

In some embodiments, the delivery vehicle is a polymer-based particle, i.e., a polyplex. Suitable polyplex carriers comprise cationic polymers such as polyethylenimine (PEI), and/or cationic polymers conjugated to neutral polymers, like polyethylene glycol (PEG) and cyclodextrin. Other suitable PEI conjugates to facilitate nucleic acid molecule or expression vector delivery in accordance with the methods described herein include, without limitation, PEI-salicylamide conjugates and PEI-steric acid conjugate. Other synthetic cationic polymers suitable for use as a delivery vehicle material include, without limitation, poly-L-lysine (PLL), polyacrylic acid (PAA), polyamideamine-epichlorohydrin (PAE) and poly[2-(dimethylamino)ethyl methacrylate] (PDMAEMA). Natural cationic polymers suitable for use as delivery vehicle material include, without limitation, chitosan, poly(lactic-co-glycolic acid) (PLGA), gelatin, dextran, cellulose, and cyclodextrin.

Solid, inorganic materials suitable for nanoparticle delivery vehicles to facilitate nucleic acid molecule or an expression vector delivery to cells include gold nanoparticles, calcium phosphate nanoparticles, cadinum (quantum dots) nanoparticles, and iron oxide nanoparticles.

Another aspect of the present invention is a host cell comprising one or more vector encoding the NorA peptide inhibitor, NorA peptide inhibitor protein scaffolds, or NorA antibody-based molecule as described herein. The NorA protein inhibitors and antibody-based molecules described herein can optionally be produced by a cell line, a mixed cell line, an immortalized cell or clonal population of immortalized cells, as well known in the art (see e.g., Ausubel et al., ed., Current Protocols in Molecular Biology, John Wiley & Sons, Inc., NY, N.Y. (1987-2001); Sambrook et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Colligan et al., eds., Current Protocols in Immunology, John Wiley & Sons, Inc., NY (1994-2001); Colligan et al., Current Protocols in Protein Science, John Wiley & Sons, NY, N.Y., (1997-2001), which are hereby incorporated by reference in their entirety).

In some embodiments, the host cell chosen for expression may be of mammalian origin. Suitable mammalian host cells include, without limitation, COS-1 cells, COS-7 cells, HEK293 cells, BHK21 cells, CHO cells, BSC-1 cells, HeG2 cells, SP2/0 cells, HeLa cells, mammalian myeloma cells, mammalian lymphoma cells, or any derivative, immortalized or transformed cell thereof. Other suitable host cells include, without limitation, yeast cells, insect cells, and plant cells. Alternatively, the host cell may be selected from a species or organism incapable of glycosylating polypeptides, e.g., a prokaryotic cell or organism, such as BL21, BL21(DE3), BL21-GOLD(DE3), XL1-Blue, JM109, HMS174, HMS174(DE3), and any of the natural or engineered E. coli spp, Klebsiella spp., or Pseudomonas spp strains.

### Pharmaceutical Compositions and Methods of Use

The NorA peptide inhibitor, NorA peptide inhibitor protein scaffolds, NorA antibody-based molecule or polynucleotide encoding these NorA proteins as disclosed herein are advantageously administered as compositions. In an embodiment, such compositions are pharmaceutical compositions comprising an active therapeutic agent (i.e., NorA peptide inhibitor, NorA peptide inhibitor protein scaffolds, or NorA antibody-based molecule) and one or more of a variety of other pharmaceutically acceptable components. See REMINGTON: THE SCIENCE AND PRACTICE OF PHARMACY (21^{st} Edition) (2005) (Troy, D. B. et al. (Eds.) Lippincott Williams & Wilkins (Publs.), Baltimore MD), which is hereby incorporated by reference in its entirety. The preferred form depends on the intended mode of administration and therapeutic application. The compositions can also include, depending on the formulation desired, pharmaceutically acceptable, non-toxic carriers, excipients, diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition, and which are vehicles commonly used to formulate pharmaceutical compositions for animal or human administration. The diluent is selected to not affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, or non-toxic, nontherapeutic, non-immunogenic stabilizers and the like. Examples of suitable aqueous and non-aqueous carriers which may be employed in the pharmaceutical compositions of the present invention include water, saline, phosphate-buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well-known in the pharmaceutical arts.

It is contemplated here that the compositions may include any suitable dose of the active therapeutic agent (i.e., NorA peptide inhibitor, NorA peptide inhibitor protein scaffolds, or NorA antibody-based molecule) which is effective for the disclosed treatments. In one embodiment, the active therapeutic agent is present in a concentration of 5 mg/ml or more, such as 10, 15, 20, 25, 30, 35, 40, 45, 50, 55, 60, 65, 70, 75, 80, 85, 90, 95, 100, 150, 200, 250, or 300 mg/ml or more. In one embodiment, the active therapeutic agent is present within the composition in an amount of between 5 mg/ml and 50 mg/ml. In another embodiment, the active therapeutic agent is present within the composition in an amount of between 50 mg/ml and 300 mg/ml, for instance between 50 mg/ml and 250 mg/ml, between 50 mg/ml and 200 mg/ml, between 50 mg/ml and 150 mg/ml, between 50 mg/ml and 100 mg/ml, between 75 mg/ml and 300 mg/ml, between 75 mg/ml and 250 mg/ml, between 75 mg/ml and 200 mg/ml, between 75 mg/ml and 150 mg/ml, between 100 mg/ml and 300 mg/ml, between 100 mg/ml and 250 mg/ml, between 100 mg/ml and 200 mg/ml, or between 100 mg/ml and 150 mg/ml.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

The compositions may also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Suitability for carriers and other components of pharmaceutical compositions is determined based on the lack of significant negative impact on the desired biological properties of the active antibody-based molecule of the present invention (e.g., less than a substantial impact (e.g., 10% or less relative inhibition, 5% or less relative inhibition, etc.) on antigen binding).

The pharmaceutical compositions of the present invention may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present invention may also comprise isotonicity agents, such as sugars, polyalcohols, such as mannitol, sorbitol, glycerol or sodium chloride in the compositions.

The pharmaceutical compositions of the present invention may also comprise one or more additives that promote formulation stability or promote lower viscosity while supporting higher loading of the active therapeutic agent (i.e., NorA peptide inhibitor, NorA peptide inhibitor protein scaffolds, or NorA antibody-based molecule). Exemplary additives include one or more amino acids or salts thereof as identified in PCT Publ. No. WO 2011/104381 to Novo Nordisk; Falconer et al., “Stabilization of a Monoclonal Antibody During Purification and Formulation by Addition of Basic Amino Acid Excipients,” J Chem Technol Biotechnol 86: 942-948 (2011); and Feng et al., “Screening of Monoclonal Antibody Formulations Based on High-throughput Thermostability and Viscosity Measurements: Design of Experiment and Statistical Analysis,” J Pharm Sci. 100(4):1330-40 (2011), each of which is hereby incorporated by reference in its entirety. Suitable amino acids include, without limitation, Gly, His, Arg, Lys, Glu, Iso, Asp, Trp, Thr, Pro, and combinations thereof, which are present in a concentration of between 0 and 100 mM, such as 50 mM, 40 mM, 35 mM, 33 mM, 30 mM, 25 mM or lower.

The pharmaceutical compositions of the present invention may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The antibodies of the present invention may be prepared with carriers that will protect the antibodies against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well-known in the art. Methods for the preparation of such formulations are generally known to those skilled in the art. See, e.g., SUSTAINED AND CONTROLLED RELEASE DRUG DELIVERY SYSTEMS, J. R. Robinson, ed., Marcel Dekker, Inc., New York, 1978.

In one embodiment, the antibodies of the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art.

Pharmaceutical compositions for injection must typically be sterile and stable under the conditions of manufacture and storage. The composition may be formulated as a solution, microemulsion, liposome, or other ordered structure suitable to achieve high drug concentration. The carrier may be an aqueous or non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

For parenteral administration, agents of the present disclosure are typically formulated as injectable dosages of a solution or suspension of the substance in a physiologically acceptable diluent with a pharmaceutical carrier that can be a sterile liquid such as water, oil, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin. Peanut oil, soybean oil, and mineral oil are all examples of useful materials. In general, glycols, such as propylene glycol or polyethylene glycol, are preferred liquid carriers, particularly for injectable solutions. Agents of the invention can be administered in the form of a depot injection or implant preparation which can be formulated in such a manner as to permit a sustained release of the active ingredient.

An exemplary composition comprises an scFv at about 5 to 50 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles, such as polylactide, polyglycolide, or copolymer, for enhanced adjuvant effect (Langer, et al., Science 249:1527 (1990); Hanes, et al., Advanced Drug Delivery Reviews 28:97-119 (1997), which are hereby incorporated by reference in their entirety). Additional formulations suitable for other modes of administration include oral, intranasal, and pulmonary formulations, suppositories, and transdermal applications.

Another aspect of the present disclosure is directed to a combination therapeutic. This combination therapeutic comprises an antibiotic and/or biocide and a NorA peptide inhibitor, NorA peptide inhibitor protein scaffold, or NorA antibody-based molecule.

In one embodiment, the antibiotic and/or biocide and the NorA peptide inhibitor, NorA peptide inhibitor protein scaffold, or NorA antibody-based molecule are formulated in a single composition.

In one embodiment, the antibiotic and/or biocide and the NorA peptide inhibitor, NorA peptide inhibitor protein scaffold, or NorA antibody-based molecule are formulated in separate compositions.

Suitable antibiotics and/or biocides for inclusion in the combination therapeutic as disclosed herein include, without limitation, fluoroquinolones, quaternary ammonium compounds, chloramphenicol, and cetrimide. In one embodiment, the antibiotic of the combination therapeutic is a fluoroquinolone selected from the group consisting of norfloxacin, enoxacin, ciprofloxacin, difloxacin, fleroxacin, lomefloxacin, pefloxacin, sparfloxacin, temafloxacin, tosufloxacin, levofloxacin, moxifloxacin, ofloxacin, gemifloxacin, and delafloxacin.

In one embodiment, the combination therapeutic as disclosed herein comprises the NorA peptide inhibitor comprising the amino acid sequence of X₁YYX₄X₅WRX₈X₉GX₁₁X₁₂ (SEQ ID NO:1), wherein X₁, X₄, X₅, X₈, X₉, X₁₁, X₁₂ are selected from any amino acid residue.

In another embodiment, the combination therapeutic as disclosed herein comprises the NorA peptide inhibitor comprising the amino acid sequence of X₁X₂X₃X₄X₅WRX₈X₉X₁₀X₁₁X₁₂ (SEQ ID NO: 27), wherein X₁ is Y, I, or V; X₂ is Y, F, or W; X₃ is Y, F, I, L, M, V, or W; X₄ is Y or W; X₅ is A, L, S, or V; X₈ is V, A, D, F, G, H, I, L, M, R, W, or Y; X₉ is G, F, P, S, or T; X₁₀ is G, A, F, I, L, N, Q, S, T, V, or Y; X₁₁ is Y, A, C, F, H, I, L, M, R, S, T, V, or W; and X₁₂ is W, F, L, or Y.

In any embodiment, the NorA peptide inhibitor of the combination therapeutic comprises an amino acid sequence selected from SEQ ID NO 2, SEQ ID NO: 3, and SEQ ID NO: 4.

Another aspect of the present disclosure is directed to a method of treating a Staphylococcus aureus infection in a subject. This method involves administering, to a subject having a S. aureus infection, an antibiotic or biocide and the pharmaceutical composition comprising a NorA peptide inhibitor, a NorA peptide inhibitor protein scaffold, or a NorA antibody-based molecule as disclosed herein. In accordance with this method, the pharmaceutical composition is administered in an amount effective to treat the S. aureus infection in the subject. In any embodiment, the S. aureus infection is a methicillin-resistant or a methicillin-sensitive S. aureus infection.

Another aspect of the present disclosure is directed to a method of potentiating the therapeutic efficacy of an antibiotic or biocide in a subject having a Staphylococcus aureus infection. This method involves administering, to a subject having a S. aureus infection and exhibiting NorA mediated antibiotic or biocide resistance, an antibiotic or biocide and the pharmaceutical composition comprising a NorA peptide inhibitor, a NorA peptide inhibitor protein scaffold, or a NorA antibody-based molecule as disclosed herein. In accordance with this method, the pharmaceutical compositions are administered in an amount effective to potentiate the therapeutic efficacy of the antibiotic or biocide in the subject. In any embodiment, the S. aureus infection is a methicillin-resistant or a methicillin-sensitive S. aureus infection.

In one embodiment of this aspect of the disclosure, the antibiotic or biocide and pharmaceutical composition are administered concurrently

In another embodiment, the antibiotic or biocide and pharmaceutical composition are administered sequentially.

In accordance with the methods disclosed herein, a lower dose of antibiotic or biocide is administered to said subject as compared to when the antibiotic or biocide is administered as a monotherapy. As disclosed supra, suitable antibiotic or biocide for use in the methods of the disclosure include, without limitation fluoroquinolones, quaternary ammonium compounds, chloramphenicol, and cetrimide. Suitable fluoroquinolones include, without limitation, norfloxacin, enoxacin, ciprofloxacin, difloxacin, fleroxacin, lomefloxacin, pefloxacin, sparfloxacin, temafloxacin, tosufloxacin, levofloxacin, moxifloxacin, ofloxacin, gemifloxacin, and delafloxacin.

In one embodiment, the pharmaceutical composition administered in the methods disclosed herein comprises the NorA peptide inhibitor comprising the amino acid sequence of X₁YYX₄X₅WRX₈X₉GX₁₁X₁₂ (SEQ ID NO:1), wherein X₁, X₄, X₅, X₈, X₉, X₁₁, X₁₂ are selected from any amino acid residue.

In another embodiment, the pharmaceutical composition administered in the methods disclosed herein comprises the NorA peptide inhibitor comprising the amino acid sequence of X₁X₂X₃X₄X₅WRX₈X₉X₁₀X₁₁X₁₂ (SEQ ID NO: 27), wherein X₁ is Y, I, or V; X₂ is Y, F, or W; X₃ is Y, F, I, L, M, V, or W; X₄ is Y or W; X₅ is A, L, S, or V; X₈ is V, A, D, F, G, H, I, L, M, R, W, or Y; X₉ is G, F, P, S, or T; X₁₀ is G, A, F, I, L, N, Q, S, T, V, or Y; X₁₁ is Y, A, C, F, H, I, L, M, R, S, T, V, or W; and X₁₂ is W, F, L, or Y.

In any embodiment, the NorA peptide inhibitor of the pharmaceutical composition comprises an amino acid sequence selected from SEQ ID NO 2, SEQ ID NO: 3, and SEQ ID NO: 4.

The pharmaceutical composition can be administered by parenteral, topical, oral or intranasal means for therapeutic treatment. Intramuscular injection (for example, into the arm or leg muscles) and intravenous infusion are preferred methods of administration of the molecules of the present invention. In one embodiment, a pharmaceutical composition is administered parenterally. The phrases “parenteral administration” and “administered parenterally” as used herein denote modes of administration other than enteral and topical administration, usually by injection, and include epidermal, intravenous, intramuscular, intraarterial, intrathecal, intracapsular, intracranial, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection, subcutaneous and infusion. In one embodiment that pharmaceutical composition is administered by intravenous or subcutaneous injection or infusion.

In accordance with all of the methods described herein a “subject” refers to any animal. In some embodiments, the subject is a mammal. Exemplary mammalian subjects include, without limitation, humans, non-human primates, dogs, cats, rodents (e.g., mouse, rat, guinea pig), horses, cattle and cows, sheep, and pigs. In some embodiments, the subject is a human. In some embodiments, the animal is a domesticated pet. In some embodiments, the animal is livestock.

Another aspect of the disclosure is directed to a method of diagnosing a S. aureus infection in a subject. This method comprises contacting a sample from a subject having a S. aureus infection with a NorA peptide inhibitor, a NorA peptide inhibitor protein scaffold, or a NorA antibody-based molecule as disclosed herein, under conditions effective to detect NorA expression in the sample. The method further involves detecting NorA expression in said sample based on said contacting, and diagnosing the S. aureus infection in the subject based on said detecting. In accordance with this method, the NorA peptide inhibitor, NorA peptide inhibitor protein scaffold, or NorA antibody-based molecule is coupled to a detectable label to facilitate detection using standard methods and assays known in the art. The label can be any detectable moiety known and used in the art. Suitable labels include, without limitation, radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

In accordance with this method, a resistant form of S. aureus infection is diagnosed when high levels of NorA expression are detected in the sample. The expression levels of NorA can be quantified and categorized as high, medium, or low by comparison to appropriate reference samples. In accordance with this methods, detecting a treatment resistant form of S. aureus infection in the subject will guide the course of treatment for the subject. For example, if a treatment resistant form of S. aureus is detected, the course of treatment may involve administration of a combination therapeutic as disclosed herein.

Another aspect of the disclosure is directed to a method of detecting NorA in a non-clinical biological sample. This method involves contacting the sample with a NorA peptide inhibitor, a NorA peptide inhibitor protein scaffold, or a NorA antibody-based molecule, under conditions effective to detect NorA expression in the sample, and detecting NorA expression in said sample based on said contacting. In some embodiment, this method further involves quantifying levels of NorA expression in the sample based on said detecting. In accordance with this method, the NorA peptide inhibitor, NorA peptide inhibitor protein scaffold, or NorA antibody-based molecule is coupled to a detectable label to facilitate detection using standard methods and assays known in the art.

### EXAMPLES

The following examples are provided to illustrate embodiments of the present invention but are by no means intended to limit its scope.

### Materials & Methods for Examples 1-5

### NorA Expression and Purification

The S. aureus NorA sequence (Uniprot accession code Q5HHX4, which is hereby incorporated by reference in its entirety) was cloned into a pET28 vector (Novagen) with a carboxy-terminal decahistidine tag. The construct was transformed into C43 (DE3) E. coli and expressed using the autoinduction method (Studier, “Protein Production by Auto-induction in High Density Shaking Cultures,” Protein Expr Purif 41:207-234 (2005), which is hereby incorporated by reference in its entirety) with minor modifications (Karpowich et al., “ATP Binding Drives Substrate Capture in an ECF Transporter by a Release-and-catch Mechanism,” Nat Struct Mol Biol 22:565-571 (2015), which is hereby incorporated by reference in its entirety). Briefly, cells were grown at 32° C. in ZYP-5052 media supplemented with 1 mM MgSO₄. At an OD₆₀₀ₙₘ of 0.5, the temperature was reduced to 20° C. and cultures were grown for an additional 18-20 hr. Cells were harvested by centrifugation and stored at −80° C.

Cell pellets were resuspended and lysed in 40 mM Tris pH 8.0, 400 mM NaCl, and 10% glycerol and the resultant lysate was clarified by centrifugation. The membrane fraction was harvested by centrifuging for 3 hr at 35,000 RPM using a Beckman ultracentrifugation equipped with a Type 45 Ti fixed-angle rotor. Membranes were resuspended in 20 mM Tris pH 8.0, 300 mM NaCl, 10% glycerol, and 10 mM imidazole and solubilized with 1% (w/v) lauryl maltose neopentyl glycol (LMNG, Anatrace). NorA was purified using immobilized metal-affinity chromatography (IMAC). In brief, NorA was bound to cobalt affinity resin (ThermoFisher Scientific) and successively washed with IMAC Buffer (20 mM Tris pH 8.0, 200 mM NaCl, 10% glycerol, and 0.2% (w/v) LMNG) containing 25 mM and 50 mM imidazole and eluted with 400 mM imidazole. NorA eluate fractions were dialyzed into size exclusion chromatography (SEC) buffer (20 mM Tris pH 7.5 and 100 mM NaCl) and incubated overnight with PMAL-C8 amphipol (Anatrace). For cryo-EM, the sample was incubated with Bio-beads (Bio-Rad) and subsequently purified on a Superdex 200 column (GE healthcare) equilibrated in SEC buffer. Peak fractions were pooled, concentrated with a 10 kDa centrifugal concentrator (Millipore), and used immediately or flash frozen and stored at −80° C. For Fab screening, LMNG-purified NorA was reconstituted into lipid nanodiscs using MSP1E3D1 following a published protocol (Sauer et al.. “Structural Basis for the Reaction Cycle of DASS Dicarboxylate Transporters,” Elife 9:e61350 (2020), which is hereby incorporated by reference in it's entirety) and the efficiency of the reconstitution was examined by SDS-PAGE and negative stain electron microscopy.

### Generation of Synthetic Fabs

Sorting of an antibody phage-display library using nanodisc-embedded NorA prepared as described above was performed as previously described (Fellouse et al., “High-throughput Generation of Synthetic Antibodies From Highly Functional Minimalist Phage-displayed Libraries,” J Mol Biol 373:924-940 (2007); Dominik & Kossiakoff, “Phage Display Selections for Affinity Reagents to Membrane Proteins in Nanodiscs,” Methods Enzymol 557:219-245 (2015), which are hereby incorporated by reference in their entirety) with slight modifications. In each round, phage solution in 50 mM Tris-HCl pH 7.5, 100 mM NaCl, 1% BSA, and 0.1 mM TCEP was first incubated with 4 μM biotinylated nanodiscs loaded with lipid but without NorA (“empty nanodiscs”) complexed with streptavidin-coated magnetic beads (catalog number Z5481, Promega). Unbound phage particles were recovered and then incubated with 100 nM NorA in biotinylated nanodiscs in solution, and phages bound to NorA were mixed with streptavidin-coated magnetic beads. Using a Kingfisher instrument (Thermo Fisher), the beads were captured, washed in the same buffer for a total of five times, then the bound phages were eluted in 100 mM glycine-HCl (pH 2.2). The elution solution was immediately neutralized with 2 M Tris-HCl (pH 8.0). A total of four rounds of library sorting were performed. Enriched clones were individually tested using phage ELISA using nanodisc-embedded NorA and empty nanodiscs immobilized in the wells of 96-well Maxisorp plates precoated with streptavidin as described previously (Fellouse et al., “High-throughput Generation of Synthetic Antibodies From Highly Functional Minimalist Phage-displayed Libraries,” J Mol Biol 373:924-940 (2007); Sidhu et al., “Phage Display for Selection of Novel Binding Peptides,” Methods Enzymol 328:333-363 (2000), which are hereby incorporated by reference in their entirety).

### Fab Expression and Purification

Fabs were subcloned into a modified pTac expression vector (Burioni et al., “A Vector for the Expression of Recombinant Monoclonal Fab Fragments in Bacteria,” J Immunol Methods 217:195-199 (1998), which is hereby incorporated by reference in its entirety) and subsequently rigidified in the hinge-region of the heavy-chain (Bailey et al., “Locking the Elbow: Improved Antibody Fab Fragments as Chaperones for Structure Determination,” J Mol Biol 430:337-347 (2018), which is hereby incorporated by reference in its entirety) by site-directed mutagenesis. A negative-control Fab (Fab_{control}) construct comprised of undifferentiated, polyserine CDR sequences was not rigidified and contained a C-terminal Avi-tag on the heavy chain. Each Fab was transformed into the 55244 strain of E. coli (ATCC) and expressed in TBG media for 22 hr at 30° C. Cell pellets were harvested by centrifugation and resuspended in running buffer (20 mM sodium phosphate pH 7.0) supplemented with 1 mg mlV hen egg lysozyme (Sigma). After cell lysis and the removal of cell debris by centrifugation, the supernatant was loaded onto a protein G column (GE Healthcare) equilibrated in running buffer. Following elution from the column with 100 mM glycine (pH 2.7), the Fabs samples were immediately neutralized with 2 M Tris buffer (pH 8). Peak fractions were pooled, flash-frozen, and stored at −80° C. until use.

### Microscale Thermophoresis Binding Assays

Amphipol-purified NorA underwent an additional round of SEC to remove free amphipol. Purified Fabs were prepared as described above and labelled with a primary amine-reactive 647 nm fluorophore (Nanotemper) according to the manufacturer's instructions. Efficient labelling was verified spectroscopically by measuring absorbance at 280 and 650 nm. Microscale thermophoresis (MST) experiments were performed on a Monolith NT.115 Pico instrument (Nanotemper Technologies). Labelled Fab at 40 nM final concentration was mixed with NorA in 20 mM Tris pH 7.0, 100 mM NaCl, and 0.025% (w/v) Tween-20. All samples were incubated for 15 min and loaded into premium glass capillaries. The normalized fluorescent signal (Fₙₒᵣₘ) was plotted as a function of ligand concentration and fit with a non-linear sigmoidal regression model (GraphPad Prism) using a Hill slope equal to 1.0 to obtain the K_{d} values. The error in K_{d} values for Fab25 (1.2±0.7 μM) and Fab36 (140±20 nM) represent the average and standard deviation of two independent runs with three replicates per independent run. The error range in the K_{d} value for Fab36^{W133A} (2 to 4 μM) corresponds to the 95% confidence interval of the non-linear fit. The Fab36^{R134A} binding curve could not be accurately fit and the K_{d} value was estimated to be greater than the highest concentration of NorA in the experiment (>14 μM). MST binding experiments with Fab36^{W133A} and Fab36^{R134A} were collected with three replicates each.

Competitive peptide binding experiments were performed with MST using fluorescently labelled Fab36 as described above. These experiments used fixed concentrations of NorA in PMAL-C8 amphipol at 6 μM and Fab36 at 40 nM, and a variable concentration of NPI-1 from 1.5 nM to 50 μM. Competitive peptide binding experiments were collected with three replicates. The superimposed solid line on the competition MST dataset was calculated using an EC₅₀ value at 6.286 mM determined from the FITC-NPI-1 binding constant to NorA (Nikolovska-Coleska et al., “Development and Optimization of a Binding Assay for the XIAP BIR3 Domain Using Fluorescence Polarization,” Anal. Biochem. 332:261-273 (2004); Huang, X. Y., “Fluorescence Polarization Competition Assay: The Range of Resolvable Inhibitor Potency is Limited by the Affinity of the Fluorescent Ligand,” J Biomol Screen 8:34-38 (2003), which are hereby incorporated by reference in their entirety; see also Fluorescence Polarization Experiments section below).

### Cryo-EM Sample Preparation and Data Collection

NorA-Fab25 and NorA-Fab36 complexes were prepared identically. Amphipol-purified NorA was incubated with purified Fabs at a 1:3 molar ratio for 2 hr at 4° C. and then purified further using a Superdex 200 10/300 column in SEC buffer. Formation of the NorA-Fab complexes was confirmed using SDS-PAGE and negative stain electron microscopy. Cryo-EM grids were prepared by applying 3 μL of sample at −2 mg mL⁻¹ to glow-discharged QuantiAuFoil 300-mesh R1.2/1.3 grids (Quantifoil) (Sauer et al., “Structural Basis for the Reaction Cycle of DASS Dicarboxylate Transporters,” Elife 9:e61350 (2020); Sauer et al., “Structure and Inhibition Mechanism of the Human Citrate Transporter NaCT,” Nature 591:157-161 (2021), which are hereby incorporated by reference in their entirety). The samples were blotted for 3.5 or 4 seconds under 100% humidity at 4° C. before plunge-freezing into liquid ethane using a Mark IV Vitrobot (FEI).

Cryo-EM data for NorA-Fab25 and NorA-Fab36 samples were acquired on a Titan Krios microscope (FEI) equipped with a K3 direct electron detector (Gatan), using a GIF-Quantum energy filter with a 20 eV slit width (Sauer et al.. “Structural Basis for the Reaction Cycle of DASS Dicarboxylate Transporters,” Elife 9:e61350 (2020); Sauer et al., “Structure and Inhibition Mechanism of the Human Citrate Transporter NaCT,” Nature 591:157-161 (2021), which are hereby incorporated by reference in their entirety). SerialEM (Mastronarde, “Automated Electron Microscope Tomography Using Robust Prediction of Specimen Movements,” J Struct Biol 152:36-51 (2005), which is hereby incorporated by reference in its entirety) was used for automated data collection. Movies were collected at a nominal magnification of 81,000× in SuperRes mode with a physical pixel size of 1.079 Å and dose-fractioned over 60 frames. The NorA-Fab25 and NorA-Fab36 datasets received an accumulated dose of 49 and 55.54 e⁻ Å⁻², respectively. A total of 3,102 movies were collected for NorA-Fab25 and 2,816 movies collected for NorA-Fab36.

### Cryo-EM Image Processing and Map Analysis

The NorA-Fab25 and NorA-Fab36 datasets were processed in cryoSPARC (Punjani et al., “cryoSPARC: Algorithms for Rapid Unsupervised Cryo-EM Structure Determination,” Nat Methods 14:fd290-296 (2017), which is hereby incorporated by reference in its entirety) with identical methodologies (Sauer et al., “Structural Basis for the Reaction Cycle of DASS Dicarboxylate Transporters,” Elife 9:e61350 (2020); Sauer et al., “Structure and Inhibition Mechanism of the Human Citrate Transporter NaCT,” Nature 591:157-161 (2021), which are hereby incorporated by reference in their entirety). Imported movies were motion corrected and CTF estimated, and micrographs with an overall resolution worse than 8 Å were excluded from subsequent analysis. Initial 2D class averages were generated using particles picked with an ellipse-based template. 2D classes representing NorA-Fab complexes were then used as templates for a second round of particle picking. A total of 4.1 and 3.73 million particles were selected from the NorA-Fab25 and NorA-Fab36 datasets, respectively. Particles underlying well-resolved 2D classes were used for initial ab initio model building, and all picked particles were used for the subsequent heterogenous 3D refinement. Particles from classes of the complete NorA-Fab complexes or NorA-Fab complexes apparently lacking the Fab constant domain were retained for subsequent rounds of ab initio model building and heterogeneous 3D classification. After multiple rounds, a non-uniform 3D refinement step was used to generate final 3.74 Å and 3.16 Å maps for NorA-Fab25 and NorA-Fab36, respectively, as assessed using the gold standard Fourier shell correlation (FSC). Final maps were sharpened using Phenix.auto_sharpen (Adams et al., “PHENIX: A Comprehensive Python-based System for Macromolecular Structure Solution,” Acta Crystallogr D Biol Crystallogr 66:213-221 (2010), which is hereby incorporated by reference in its entirety) for 3D model building.

The source of directional streaking in each NorA-Fab map was investigated by determining directional FSC curves (Tan et al., “Addressing Preferred Specimen Orientation in Single-particle Cryo-EM Through Tilting,” Nat Methods 14:793-796 (2017), which is hereby incorporated by reference in its entirety), calculating new maps in cisTEM (Grant et al., “cisTEM, User-friendly Software for Single-particle Image Processing,” Elife 7 (2018), which is hereby incorporated by reference in its entirety), and performing 3D variability analysis (3DVA) in cryoSPARC. The directional FSC distribution revealed a minor preferred orientation bias for both NorA-Fab complexes, consistent with the preferred particle orientations shown in the angular particle distribution heatmaps. The preferred orientation was more evident in the NorA-Fab36 complex. Following the strategy of Dang et al. (“Cryo-EM Structures of the TMEM16A Calcium-activated Chloride Channel,” Nature 552:426-429 (2017), which is hereby incorporated by reference in its entirety) to examine the effect of preferred orientation on reconstruction quality, maps were also calculated in cisTEM for the NorA-Fab complexes. For each complex, the cisTEM and cryoSPARC maps were of similar overall resolution, sphericity, and map quality. 3DVA calculations were performed using 4.0 Å-filtered final maps from cryoSPARC and visualized as movies in Chimera (Pettersen et al., “UCSF Chimera—a Visualization System for Exploratory Research and Analysis,” J Comput Chem 25:1605-1612 (2004), which is hereby incorporated by reference in its entirety). In each NorA-Fab complex, a bending movement was observed at the NorA-Fab interface and the interdomain hinge-region between the variable and constant domains of the Fab. This movement was more pronounced in the NorA-Fab36 complex. Overall, this analysis revealed the directional streaking to be caused by a minor orientation bias and flexibility within the NorA-Fab complex; however, this imperfection in map quality did not influence the interpretability of the maps.

### Building of Structural Models

The NorA-Fab complex structural models were built in Coot Emsley et al., “Coot: Model-building Tools for Molecular Graphics,” Acta Crystallogr D Biol Crystallogr 60:2126-2132 (2004), which is hereby incorporated by reference in its entirety) and refined using Phenix (Adams et al., “PHENIX: A Comprehensive Python-based System for Macromolecular Structure Solution,” Acta Crystallogr D Biol Crystallogr 66:213-221 (2010), which is hereby incorporated by reference in its entirety). Backbone N- and C-terminal domain structures of LacY (PDB ID: 2CFP) and MdfA (PDB ID: 6GV1) were used as references for NorA model building. The Fab for each complex was rebuilt from a homologous Fab structure (PDB ID: 5E08) after removing the variable loop regions.

### MIC Assays in MRSA

Wild-type USA300 strain JE2 (“MRSA”) (Fey et al., “A Genetic Resource for Rapid and Comprehensive Phenotype Screening of Nonessential Staphylococcus aureus Genes,” Mbio 4:e00537-00512 (2013), which is hereby incorporated by reference in its entirety) and isogenic strains, norA::bursa (MRSA^{ΔnorA}) containing empty vector, or hemin-inducible NorA variants were grown overnight at 37° C. in TSB media supplemented with 10 μg/ml chloramphenicol and 1 μM hemin. The cultures were diluted to approximately 1×10⁸ CFU/ml and evenly spread on TSA plates supplemented with 5 μg/ml chloramphenicol and 1 μM hemin. The norfloxacin MIC test strips (Liofilchem) were placed in the center of the plate and the plates were incubated for 24 hr at 37° C. The MIC values were read at the intersection of the inhibition eclipse and the strip. At least two independent MIC experiments were acquired for each sample; reported errors reflect the standard deviation among independent experiments.

### Bacteriostatic Bactericidal Effect of Norfloxacin on MRSA

MRSA or MRSA^{ΔnorA} were cultured overnight in 5 ml TSB and diluted 1:1,000 with TSB the next morning. In a round-bottom 96-well plate, 50 μl of the diluted culture was mixed with 50 μl of TSB supplemented with norfloxacin. The final concentration of MRSA or MRSA^{ΔnorA} was ˜1×10⁶ CFU/ml and norfloxacin was 0, 12.5, or 25 μg/ml. The plate was incubated while shaking at 37° C. At 0 hr and after 1, 2, or 4 hr of incubation, CFUs of MRSA or MRSA^{ΔnorA} were enumerated by serially diluting in PBS and spot plating on TSA plates.

### Growth Inhibition Assays in MRSA

Wild-type MRSA and MRSA^{ΔnorA} containing empty vector or hemin-inducible NorA variants were grown overnight at 37° C. in TSB media supplemented with 10 μg/ml chloramphenicol. The cultures were diluted 1:1,000 in TSB supplemented with 5 μg/ml chloramphenicol and 1 μM hemin, in the presence or the absence of norfloxacin (12.5 μg/ml). OD₆₀₀ₙₘ was measured as a function of time in a BioScreen C device at 37° C. with shaking between measurements every 15 min. At least two independent experiments were acquired with two technical replicates per experiment for each sample; reported errors reflect the standard deviation among technical replicates in a representative experiment.

To test the effects of peptides, overnight cultures of wild-type MRSA and MRSA^{ΔnorA} in TSB media were diluted 1:1,000 in TSB with varying concentrations of peptides, in the presence or the absence of norfloxacin (12.5 μg/ml). All the peptide concentrations contained equal amount of the DMSO vehicle. OD₆₀₀ₙₘ was measured as a function of time in a BioScreen C device at 37° C. with shaking between measurements every 15 min. Three independent experiments were acquired; reported errors reflect the standard deviation among the independent experiments.

### Characterization of NorA and Mutant Expression Levels in MRSA

MRSA^{ΔnorA} containing empty vector or hemin-inducible NorA variants were grown overnight at 37° C. in TSB media supplemented with 5 μg/ml chloramphenicol and 2.5 μM hemin. MRSA cell pellets were lysed in 40 μg/ml lysostaphin, 20 u/ml DNase, 40 μg/ml RNase A, 1× Halt Protease Inhibitor Cocktail in 50 mM Tris pH 7.5, 10 mM MgCl₂, and 1 mM CaCl₂ in Lysing matrix B tubes and bead beat for 3 rounds at 6 m/s. After removing the cell debris by centrifuging for 10 min at 10,000 RPM in an Eppendorf 5430 R microcentrifuge, the membrane fractions were pelleted from the supernatant by centrifuging for 1 hr at 40,000 RPM in a Beckman Optima MAX-TL ultracentrifuge equipped with a TLA-100 rotor. The membrane fractions were solubilized with a solution containing 2% SDS, 1× Halt Protease Inhibitor Cocktail, and 50 mM Tris pH 6.8. Protein concentrations were determined by a BCA assay. Total protein for each sample was normalized and subsequently loaded on the SDS-PAGE gel and immunoblotted using primary antibodies to the C-terminal myc tag on NorA (Genscript) and SrtA (Mazmanian et al., “Passage of Heme-iron Across the Envelope of Staphylococcus aureus,” Science 299:906-909 (2003), which is hereby incorporated by reference in its entirety) using dilutions of 1:1,000 and 1:20,000, respectively.

NorA Resistance Assays in E. coli

Wild-type NorA harboring a C-terminal myc-tag in pTrcHis2C (Invitrogen) (Yu et al., “NorA Functions as a Multidrug Efflux Protein in Both Cytoplasmic Membrane Vesicles and Reconstituted Proteoliposomes,” J Bacteriol 184:1370-1377 (2002), which is hereby incorporated by reference in its entirety) was used to engineer NorA mutants using site-directed mutagenesis. Wild-type NorA, NorA mutants, or empty vector were transformed into BL21 DE3 E. coli and grown at 37° C. in LB media. At an OD₆₀₀ₙₘ of 1.0, the cultures were spotted in 10-fold serial dilutions on LB-agar plates infused with norfloxacin (15 nM). After overnight incubation at 37° C., plates were imaged using a gel documentation system (Bio-Rad). The serial dilution screen was performed one time; loss-of-function mutants identified from the screen were confirmed in independent E. coli serial dilution experiments and in MRSA^{ΔnorA} using MIC and growth inhibition experiments (see above).

Inhibition of NorA by Fabs in E. coli

NorA was encoded in a pTrcHis2C plasmid (ampicillin resistance) with a C-terminal myc-tag (Yu et al., “NorA Functions as a Multidrug Efflux Protein in Both Cytoplasmic Membrane Vesicles and Reconstituted Proteoliposomes,” J Bacteriol 184:1370-1377 (2002), which is hereby incorporated by reference in its entirety), while Fabs were cloned in a pBAD33 plasmid (gentamicin resistance) (Jimenez et al., “Genetics and Proteomics of Aeromonas salmonicida Lipopolysaccharide Core Biosynthesis,” J Bacteriol 191:2228-2236 (2009), which is hereby incorporated by reference in its entirety) with a C-terminal Avi-tag on the heavy chain of the Fab. Competent Top10 E. coli were co-transformed with vectors containing NorA and Fabs (Fab25, Fab36, or Fab_{control}) and grown on LB agar plates infused with carbenicillin and gentamicin. For time dependent growth curves, a single colony from the transformation was inoculated into TBG media in the presence of carbenicillin and gentamicin. The culture was grown to saturation for ˜20 hr at 30° C. Cultures were diluted by 300-fold into fresh TBG media containing carbenicillin and gentamicin, norfloxacin (0 or 1.2 μg/ml), and arabinose (0%, 0.0001%, 0.0005%, 0.0025%, 0.01%, 0.05%, 0.1%, 0.2%, 0.4%, or 0.8%). OD₆₀₀ₙₘ was measured as a function of time in a BioScreen Pro C device at 30° C. with shaking between measurements every 15 min. Four independent experiments were acquired with two technical replicates per experiment; reported errors reflect the standard deviation among the experiments. Immunoblot (IB) analyses were performed on E. coli lysates co-expressing NorA and Fabs following 24 hr of growth in TBG media in the absence of norfloxacin. Primary antibodies at 1:1,000 dilutions detected the C-terminal myc tag on NorA (Genscript) and the C-terminal Avi tag on the heavy chain of the Fab (Genscript). IB analyses were repeated two independent times.

### Synthesis of Peptides Mimicking Fab36 CDRH3

Peptides NPI-1 (NH₂-YYYYAWRVGGYWR-CONH₂, SEQ ID NO: 3) and NPI-2 (NH₂-YYYYAWEVGGYWR-CONH₂, SEQ ID NO: 27) were synthesized on Gyros Protein Technologies PurePep Chorus automated peptide synthesizer using standard Fmoc peptide synthesis on Rink amide resin (0.27 mmol/g). A fluorescent analog of NPI-1 (FITC-NPI-1: FITC-DA-YYYYAWRVGGYWR-CONH₂, SEQ ID NO: 57) was also synthesized by treating resin-bound NPI-1 with fluorescein isothiocyanate (1.25 eq.) and DIEA (2.5 eq.) in DMF for 2 hr. Synthesized peptides were globally deprotected and cleaved from resin with reagent K (82.5% TFA, 5% water, 5% phenol, 5% thioanisole, and 2.5% 1,2-ethanedithiol) for 2 hr. The mixture was concentrated, and the peptides were precipitated with diethyl ether. Peptides were purified by reverse-phase HPLC using a preparative-scale C18 column. Purity of the peptides was confirmed by analytical HPLC. Purified peptides were characterized by MALDI-TOF mass spectrometry. NPI-1: observed mass of 1802.05 [M+H]⁺; calculated mass of 1801.85 [M+H]⁺. NPI-2: observed mass of 1775.07 [M+H]⁺; calculated mass of 1774.79 [M+H]⁺. FITC-NPI-1: observed mass of 2263.76 [M+H]⁺; calculated mass of 2263.95 [M+H]⁺.

### Fluorescence Polarization Experiments

Amphipol-purified NorA underwent an additional round of SEC to remove free amphipol. Fluorescence polarization (FP) experiments were performed on a DTX 880 Multimode Detector (Beckman) at 25° C. with excitation and emission wavelengths set to 485 and 525 nm, respectively. For NorA binding assays, increasing concentrations of NorA were added to wells of 15 nM FITC-NPI-1 in 100 μL solutions of 20 mM Tris pH 7.5, 100 mM NaCl, 10% glycerol, 0.1% pluronic acid, and 1.2% DMSO on black, U-shape 96 well plates (BRANDplates). All samples were incubated for 15 min on a shaking plate. FP values as a function of NorA concentration ([L]) were fit using the following quadratic binding equation in GraphPad Prism to obtain the binding affinity (K_{d}) for FITC-NPI-1 ([P]_{T}).

$$\\ \begin{matrix}FP = \frac{{K}_{d} + [ L ] + {[ P ]}_{T} - \sqrt{{( {K}_{d} + [ L ] + {[ P ]}_{T} )}^{2} - {4 [ L ] [ P ]}_{T}}}{{2 [ L ] [ P ]}_{T}} {Y}_{max} + {Y}_{offset} & ( 1 )\end{matrix}$$

Yₘₐₓ and Y_{offset} are normalization parameters. The reported K_{d} value and error for FITC-NPI-1 reflects the average and standard deviation among seven independent experiments, each performed in triplicate.

FP competition binding experiments with norfloxacin were performed using FITC-NPI-1 as described above. These experiments used a fixed concentration of NorA in PMAL-C8 amphipol at 50 nM and FITC-NPI-1 at 15 nM with norfloxacin concentrations of 10 nM and 2 mM in 100 μL solutions of 20 mM Tris pH 7.5, 100 mM NaCl, 10% glycerol, 0.1% pluronic acid, and 1.2% DMSO. A control experiment was performed in the same manner but in the absence of NorA. Four independent trials of FP experiments were performed, each in triplicate.

### Example 1—Structures of NorA-Fab Complexes

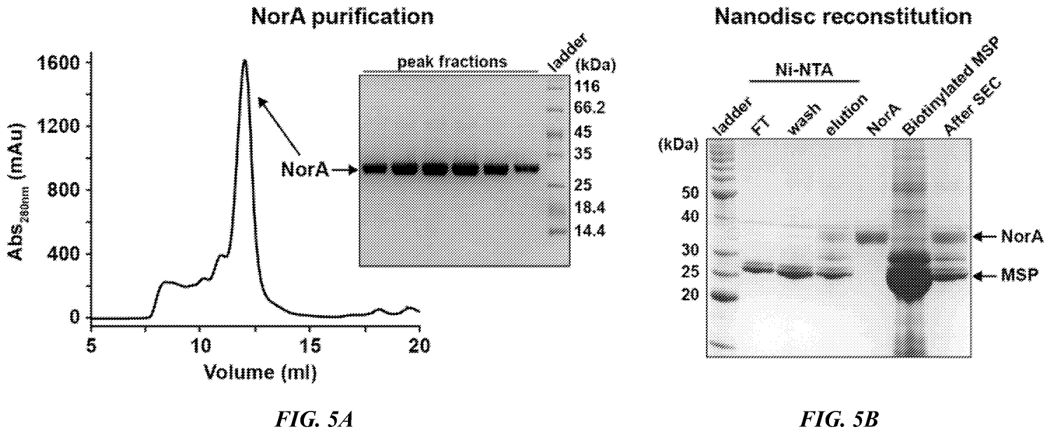

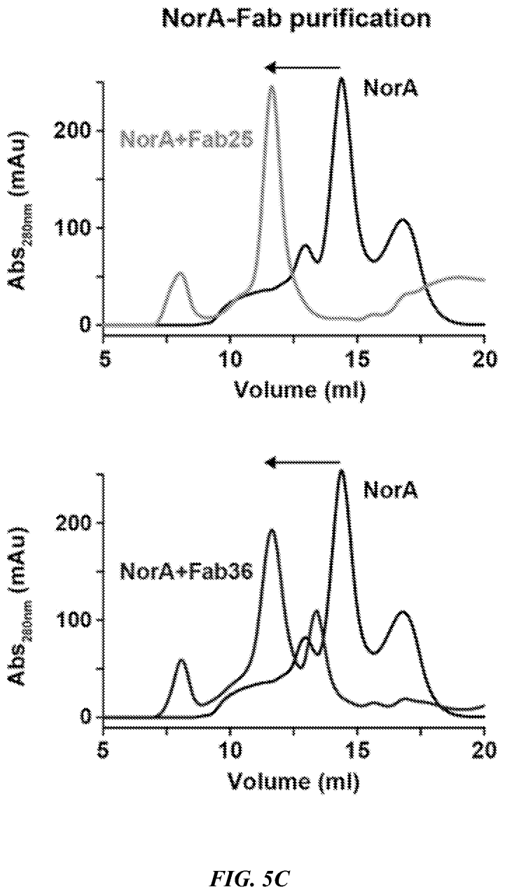

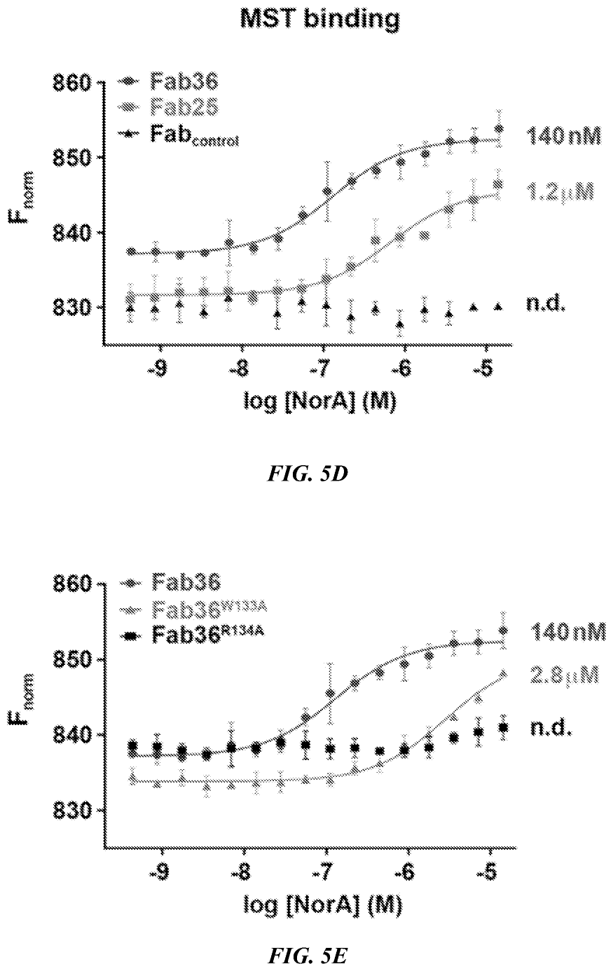

To elucidate an atomic resolution structure of NorA (42 kDa) using single particle cryo-EM, Fabs that bound to NorA were identified using phage display technology (Sidhu & Koide, “Phage Display for Engineering and Analyzing Protein Interaction Interfaces,” Curr Opin Struct Biol 17:481-487 (2007), which is hereby incorporated by reference in its entirety), which increased its effective mass to within the current limits of cryo-EM methodology (Nygaard et al., “Cryo-electron Microscopy Analysis of Small Membrane Proteins,” Curr Opin Struct Biol 64:26-33 (2020); Wu et al., “Fabs Enable Single Particle cryoEM Studies of Small Proteins,” Structure 20:582-592 (2012), which are hereby incorporated by reference in their entirety). Detergent-purified NorA was exchanged into amphipol and purified as a 1:1 complex with Fabs using size-exclusion chromatography (FIGS. 5A-5C). Subsequent screening and cryo-EM data collection of NorA-Fab complexes revealed two suitable complexes for structure determination with K_{d} values of 1.2±0.7 μM for NorA-Fab25 and 140±20 nM for NorA-Fab36 (FIG. 5D). Image processing of cryo-EM datasets generated Coulomb potential maps at resolutions of 3.74 Å for NorA-Fab25 and 3.16 Å for NorA-Fab36. While each map exhibited a minor preferred particle orientation and flexibility within the NorA-Fab complex, these did not affect the interpretability of either map. As such, the quality of the maps was relatively uniform throughout each NorA-Fab complex and allowed for unambiguous chain tracing.

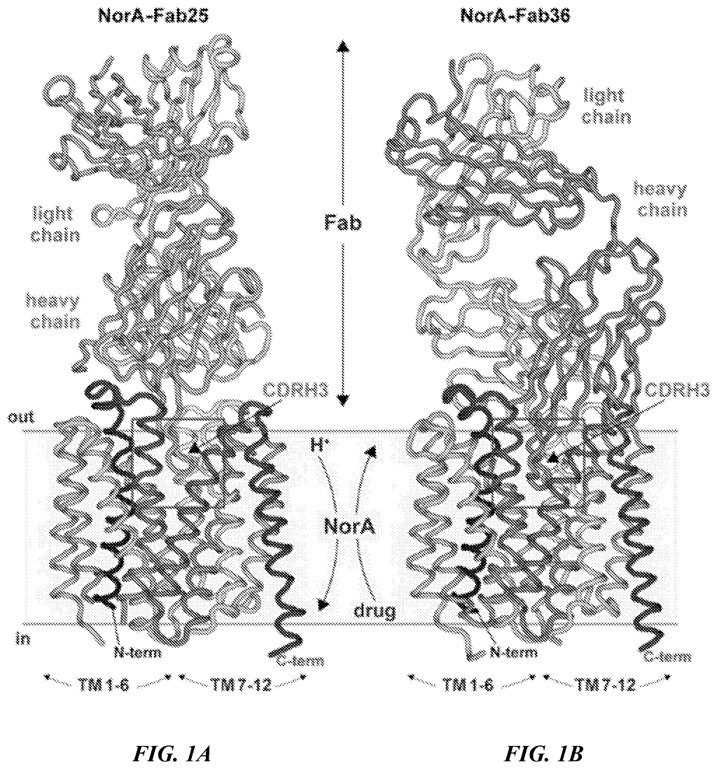

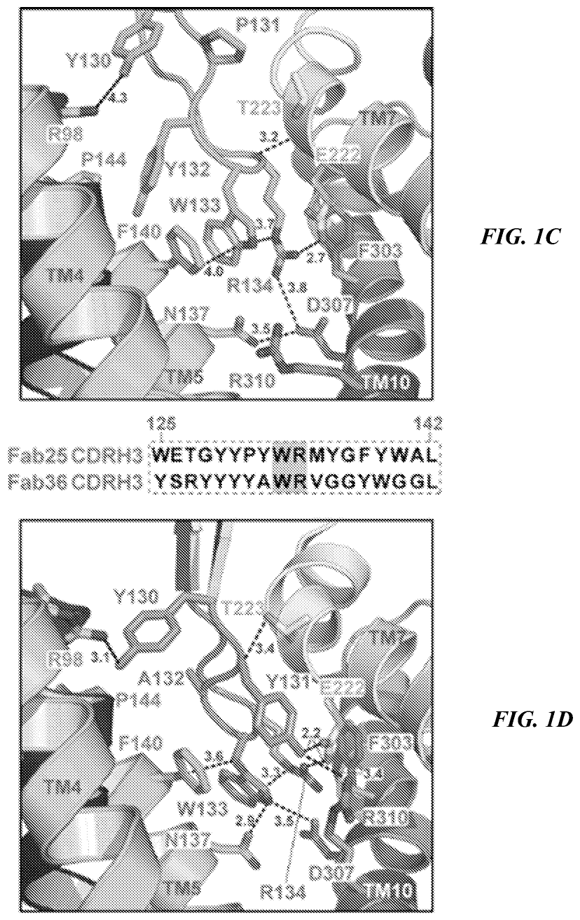

Both cryo-EM structures captured NorA in a similar outward-open conformation (1.2 Å RMSD) and in a 1:1 complex with each Fab (FIGS. 1A-1B). NorA is comprised of 12-TM a-helices arranged in two 6-TM bundles (N- and C-terminal domains) that straddle a putative substrate binding pocket. The pocket is sealed from the cytoplasmic side of the membrane by interactions among TM4, TM5, TM10, and TM11. The N- and C-terminal domains interact through interdomain contacts between TM2-TM11 and TM5-TM8. These TM pairs display characteristic hourglass shapes, which likely facilitate conformational exchange through the rocker-switch mechanism (Huang et al., “Structure and Mechanism of the Glycerol-3-phosphate Transporter From Escherichia coli,” Science 301:616-620 (2003); Abramson et al., “Structure and Mechanism of the Lactose Permease of Escherichia coli,” Science 301:610-615 (2003); Law et al., “Ins and Outs of Major Facilitator Superfamily Antiporters,” Annu Rev Microbiol 62:289-305 (2008), which are hereby incorporated by reference in their entirety). Like other homologous DHA12 drug antiporters, NorA contains characteristic MFS sequence motifs, including Motif A and Motif C that are important for conformer stability and conformational exchange, respectively (see Table 4 below) (Paulsen & Skurray, “Topology, Structure and Evolution of Two Families of Proteins Involved in Antibiotic and Antiseptic Resistance in Eukaryotes and Prokaryotes—an Analysis,” Gene 124:1-11 (1993); Jiang et al., “Structure of the YajR Transporter Suggests a Transport Mechanism Based on the Conserved Motif A,” Proc Natl Acad Sci USA 110:14664-14669 (2013); Ginn et al., “The TetA(K) Tetracycline/H(+) Antiporter From Staphylococcus aureus: Mutagenesis and Functional Analysis of Motif C,” J Bacteriol 182:1492-1498 (2000), which are hereby incorporated by reference in their entirety)

TABLE 4
Comparison of NorA with Major Facilitator
Superfamily Consensus

| | | SEQ ID | | SEQ ID |
|---|---|---|---|---|
| | Motif A | NO: | Motif C | NO: |
| NorA | GTLADKL | 58 | GFILG | 60 |
| | GKKLII | | PGIGG | |
| Consensus | GxLaDrx | 59 | GxxxG | 61 |
| | GrkxxI | | PxiGG | |

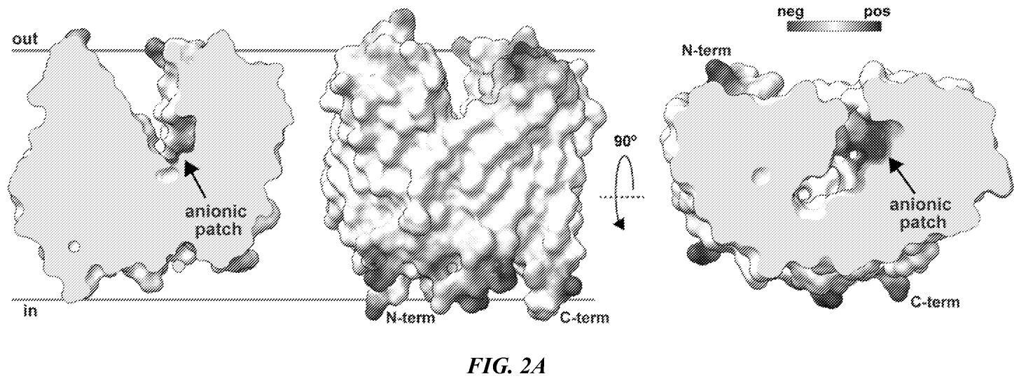

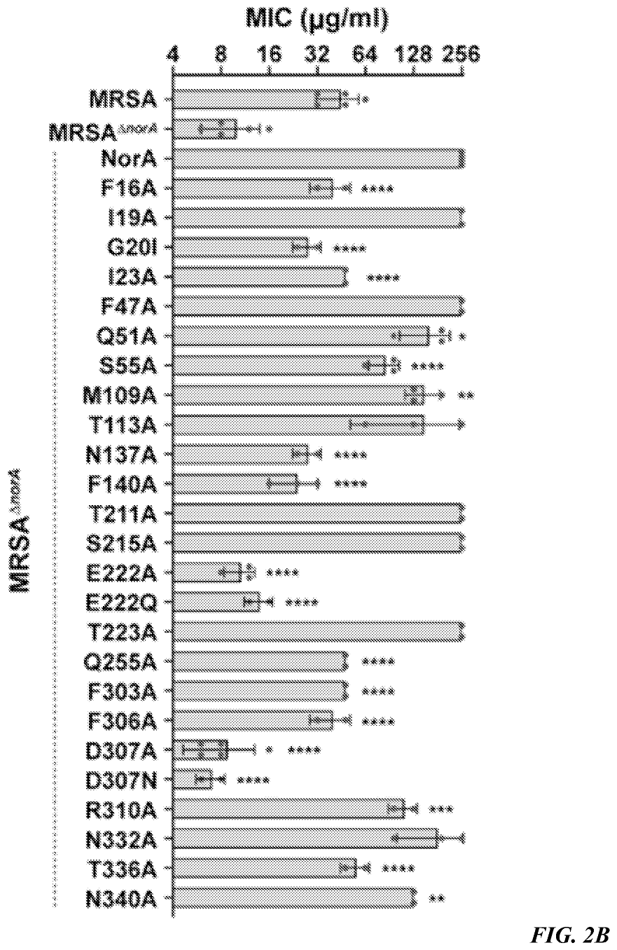

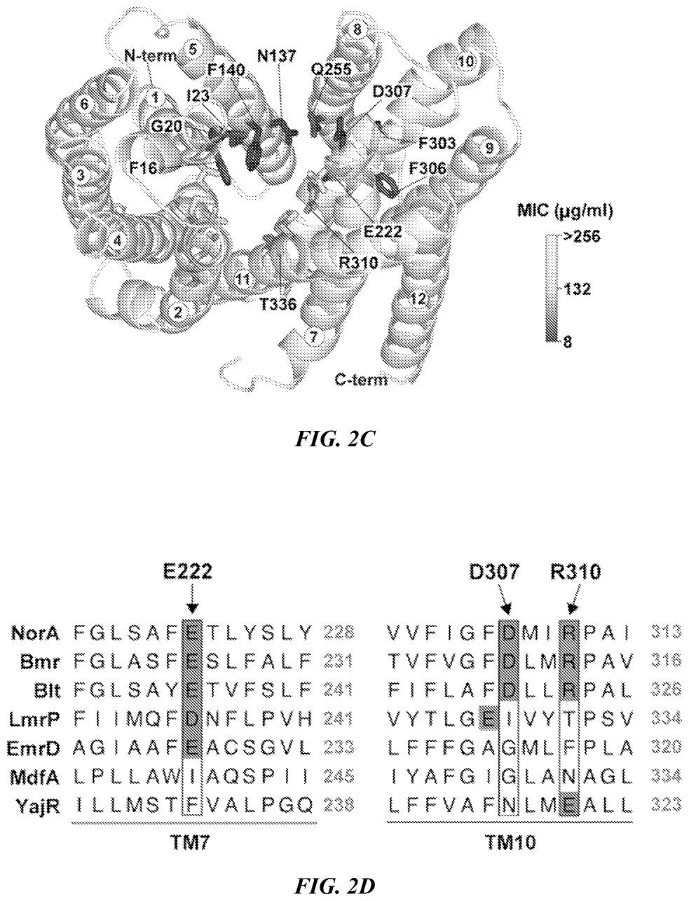

NorA's substrate binding pocket is largely hydrophobic (˜65% lipophilic residues) and contains several aromatic residues, such as Phe16, Phe140, Phe303, and Phe306, which likely facilitate binding to aromatic drugs, including fluroquinolones (Klyachko et al., “Mutations Affecting Substrate Specificity of the Bacillus subtilis Multidrug Transporter Bmr,” J Bacteriol 179:89-2193 (1997), which is hereby incorporated by reference in its entirety). The pocket also contains four ionizable residues (Arg98, Glu222, Asp307, Arg310) that create two distinct charged patches. A positively charged patch is formed by Arg98 (TM4) in the N-terminal domain and a negatively charged patch is formed by neighboring residues Glu222 (TM7) and Asp307 (TM10) in the C-terminal domain which are proximal to Arg310 (TM10) (FIGS. 1C-1D, 2A). The acidic patch may play a functional role in drug binding as several substrates of NorA carry net positive charges (Neyfakh et al., “Fluoroquinolone Resistance Protein NorA of Staphylococcus aureus is a Multidrug Efflux Transporter,” Antimicrob Agents Chemother 37:128-129 (1993), which is hereby incorporated by reference in its entirety).

### Example 2—NorA-Fab Binding Interfaces

Fab25 and Fab36 engage NorA from the extracellular side of the membrane through extensive intermolecular contacts. Most striking is the insertion of a hairpin loop, belonging to the heavy chain of CDR3 (CDRH3), into NorA's substrate binding pocket (FIGS. 1C-1D). This aromatic-rich, 18-residue loop inserts ˜15 Å into the pocket and interacts with NorA residues in the N- and C-terminal domains. Each CDRH3 loop contains a tryptophan-arginine motif, Trp133-Arg134, positioned at the tip and oriented for an intramolecular cation-π bond (FIGS. 1C-1D). On the opposite side of the arginine, the guanidinium group of Arg134 forms a second cation-7r interaction with Phe303 (TM10) of NorA. This same arginine also makes electrostatic interactions with the carboxyl groups of Glu222 (TM7) and Asp307 (TM10) in NorA. In agreement with their importance in mediating contacts in the substrate binding pocket, mutation of the tryptophan (W133A) or arginine (R134A) in Fab36 CDRH3 resulted in ˜20-fold and >100-fold reduced binding affinity to NorA (FIG. 5E). Since Glu222 and Asp307 of NorA closely interact with Arg134 of each CDRH3 loop, these binding data also indicate the two acidic residues are critical for Fab binding.

Despite the similar mode of CDRH3 hairpin insertion for Fab25 and Fab36 into NorA's substrate binding pocket, three notable structural differences were found between the complexes. First, Fab25 and Fab36 bind NorA with distinct orientations that are rotated approximately 20° relative to one another along an axis orthogonal to the membrane plane (FIGS. 1A-1B). Second, the positions of CDRH3 loops between Fab25 and Fab36 are translated by ˜5 Å relative to each other and interact with NorA differently. Namely, Trp133 of the tryptophan-arginine motif in Fab25 forms 7r-stacking interactions with Phe140 of NorA (FIG. 1C), while the indole nitrogen of Trp133 in Fab36 hydrogen bonds with the side chains of Asn137 and Asp307 of NorA (FIG. 1D). Third, Fab36 displays a more extensive binding surface with NorA (4,969 Å², sum of solvent-inaccessible surfaces) relative to the NorA-Fab25 interface (3,116 Å²), which explains the ˜9-fold tighter binding affinity (FIG. 5D-5E). A considerable portion of the additional Fab36 interaction surface is provided by an elongated lasso-shaped loop (Leu71 to Arg85 of the light chain) that traverses the upper portion of NorA's substrate binding pocket directly atop the CDRH3 hairpin loop.

### Example 3—Key Residues in NorA Mediating Drug Resistance

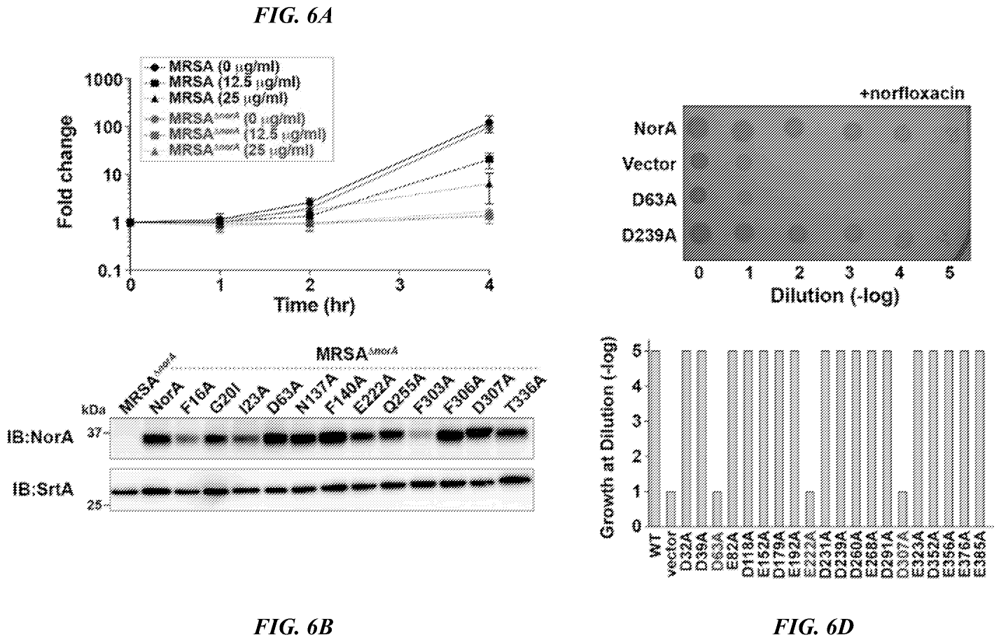

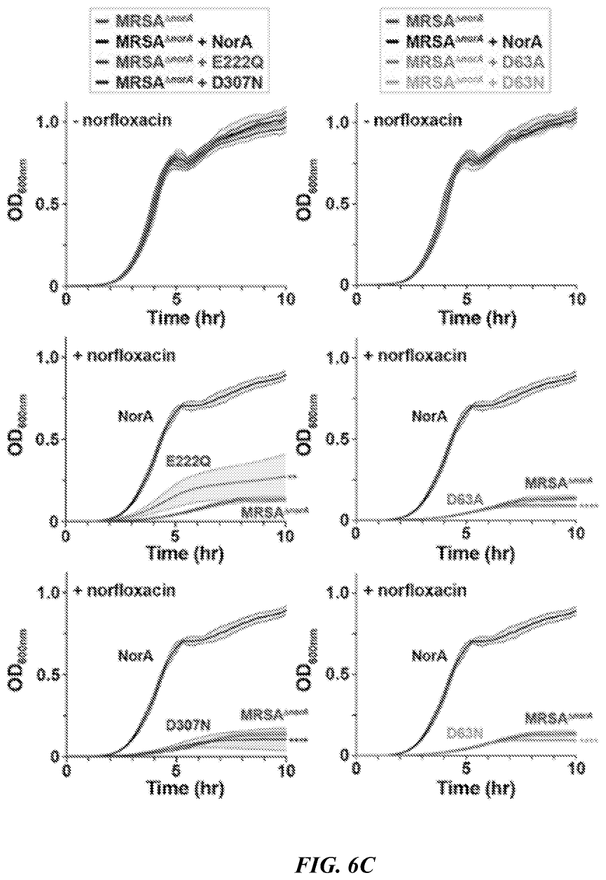

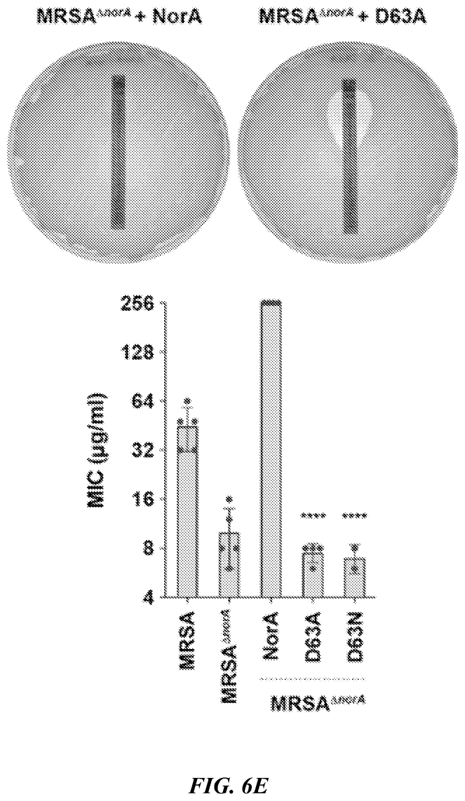

To determine functionally important residues within NorA, norfloxacin minimum inhibitory concentration (MIC) assays were performed using a MRSA strain (USA300) where the native NorA gene was disrupted by a transposon insertion (MRSA^{ΔnorA}) (Fey et al., “A Genetic Resource for Rapid and Comprehensive Phenotype Screening of Nonessential Staphylococcus aureus Genes,” Mbio 4:e00537-00512 (2013), which is hereby incorporated by reference in its entirety). Norfloxacin was found to elicit a bacteriostatic effect on the growth of MRSA and MRSA^{ΔnorA} (FIG. 6A). Wild-type NorA and 23 single-site mutations lining the substrate binding pocket were cloned into a hemin-inducible plasmid (Torres et al., “A Staphylococcus aureus Regulatory System That Responds to Host Heme and Modulates Virulence,” Cell Host Microbe 1:109-119 (2007), which is hereby incorporated by reference in its entirety) and transformed into MRSA^{ΔnorA}. Expression of wild-type NorA conferred a 20-fold greater norfloxacin MIC relative to MRSA^{ΔnorA} alone (FIG. 2B), underscoring NorA's functional role in mediating antibiotic resistance. From the panel of mutants, 11 residues that displayed a 4-fold or greater reduction in MIC relative to wild-type NorA were identified. They are: Phe16, Gly20, and Ile23 in TM1; Asn137 and Phe140 in TM5; Glu222 in TM7; Q255 in TM8; Phe303, Phe306, and Asp307 in TM10; Thr336 in TM11 (FIG. 2B). Each loss-of-function mutant was assessed for expression in MRSA^{ΔnorA} to substantiate the functional significance of the MIC measurements (FIG. 6B). When mapped onto the NorA structure, these residues outline a cluster within the substrate binding pocket that spans across the N- and C-terminal domains (FIG. 2C). Based on the spatial arrangement of these functionally important residues, this cluster is proposed to define both the norfloxacin- and proton-binding sites in NorA. Overlapping binding sites are a mechanism used by some exchangers to facilitate antiport by preventing simultaneous proton and substrate binding (Law et al., “Ins and Outs of Major Facilitator Superfamily Antiporters,” Annu Rev Microbiol 62:289-305 (2008); Schuldiner, S., “Competition as a Way of Life for H(+)-coupled Antiporters,” J Mol Biol 426:2539-2546 (2014), which are hereby incorporated by reference in their entirety). Notably, among the 11 identified residues in NorA's substrate binding pocket, Asn137, Phe140, Glu222, Phe303, and Asp307 mediated intermolecular contacts with CDRH3 of Fab25 and/or Fab36 (FIGS. 1C-1D).

Within the putative drug/proton binding site is an anionic patch comprised of Glu222 and Asp307 (FIG. 2A). Substitution of either residue to alanine ablated NorA-mediated norfloxacin resistance in MRSA^{ΔnorA} (FIG. 2B). To determine whether an ionizable carboxylate group was required for resistance at either position, MIC and growth inhibition experiments were performed in the presence of norfloxacin in MRSA^{ΔnorA} using NorA mutants E222Q and D307N. MIC measurements for E222Q and D307N revealed ablated resistance phenotypes comparable to E222A and D307A, respectively (FIG. 2B). Similarly, growth inhibition assays in the presence of norfloxacin showed loss-of-function phenotypes for E222Q and D307N (FIG. 6C). Together, these data indicate a functional role for Glu222 and Asp307 in proton-coupled transport.

Asp63 in TM2 is the third essential anionic residue in NorA, which was identified from MRSA^{ΔnorA} and E. coli resistance assays with the D63A and D63N mutants (FIGS. 6C-6E). However, unlike Glu222 and Asp307, Asp63 is not involved in proton transport due to its location outside of the membrane; instead, it is a conserved residue found within Motif A of MFS transporters that forms a hydrogen bond with the backbone amide hydrogen of Arg324 (TM11) to stabilize the outward-open conformation (Jiang et al., “Structure of the YajR Transporter Suggests a Transport Mechanism Based on the Conserved Motif A,” Proc Natl Acad Sci USA 110:14664-14669 (2013), which is hereby incorporated by reference in its entirety).

The NorA structures coupled with functional data strongly implicate Glu222 and Asp307 as the only negatively charged residues involved in the proton-coupling mechanism. These residues are conserved in NorA's closest homologues, Bmr and Blt from B. subtilis, yet are absent at corresponding positions in other DHA12 subfamily efflux pumps of known structure (EmrD, LmrP, MdfA, YajR (Jiang et al., “Structure of the YajR Transporter Suggests a Transport Mechanism Based on the Conserved Motif A,” Proc Natl Acad Sci USA 110:14664-14669 (2013); Yin et al., “Structure of the Multidrug Transporter EmrD from Escherichia coli,” Science 312:741-744 (2006); Debruycker et al., “An Embedded Lipid in the Multidrug Transporter LmrP Suggests a Mechanism for Polyspecificity,” Nat Struct Mol Biol 27:829-835 (2020); Heng et al., “Substrate-bound Structure of the E. coli Multidrug Resistance Transporter MdfA,” Cell Res 25:1060-1073 (2015), which are hereby incorporated by reference in their entirety) (FIG. 2D). These efflux pumps instead contain essential aspartate and glutamate residues for proton-coupled transport at different membrane-embedded locations (Jiang et al., “Structure of the YajR Transporter Suggests a Transport Mechanism Based on the Conserved Motif A,” Proc Natl Acad Sci USA 110:14664-14669 (2013); Fluman et al., “Dissection of Mechanistic Principles of a Secondary Multidrug Efflux Protein,” Mol Cell 47:777-787 (2012), which are hereby incorporated by reference in their entirety). This observation highlights the subtle differences in transport mechanisms even among homologous drug efflux pumps.

### Example 4—Inhibition of NorA by Fabs

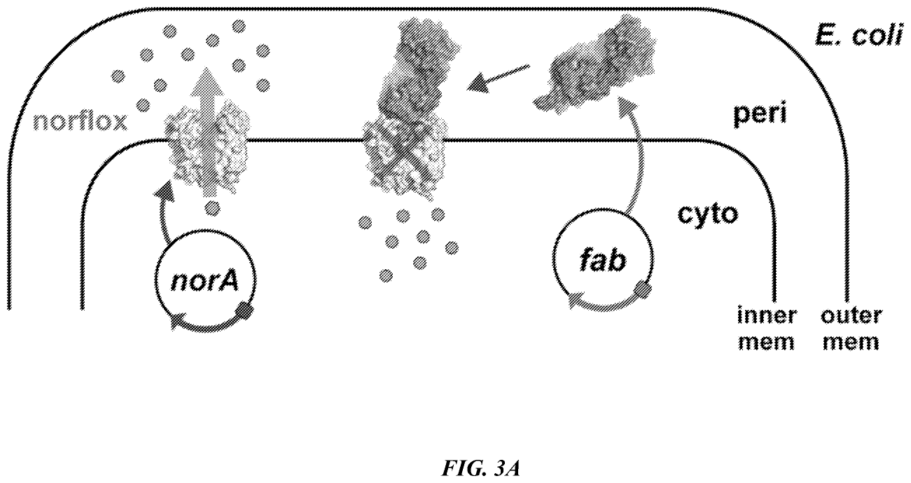

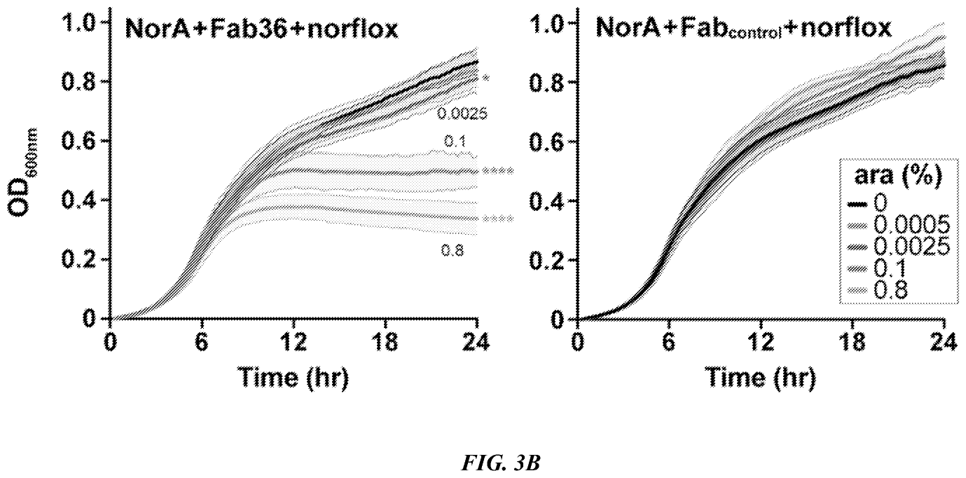

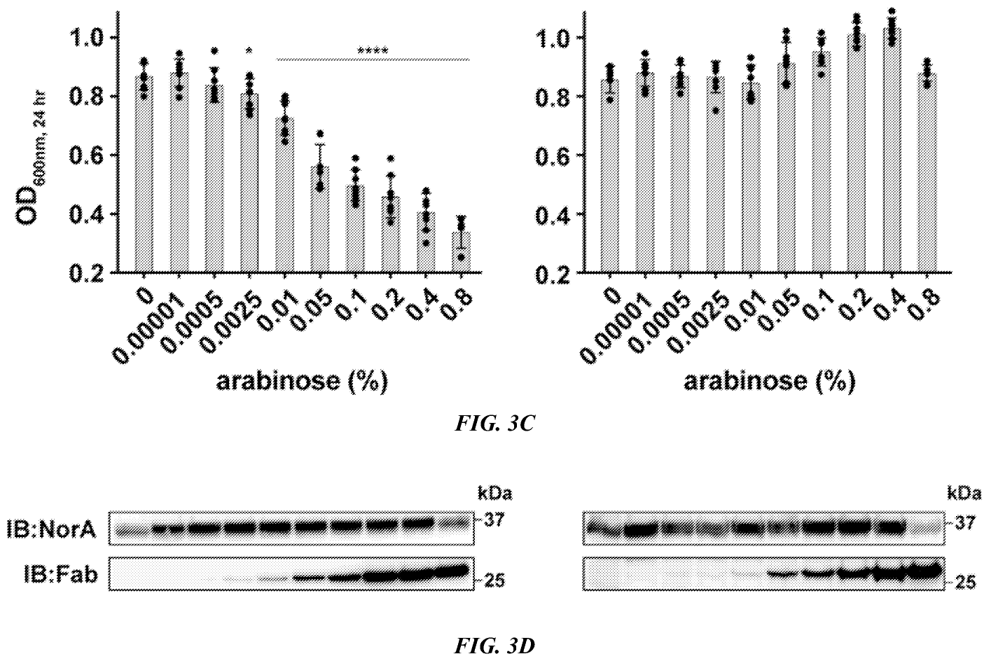

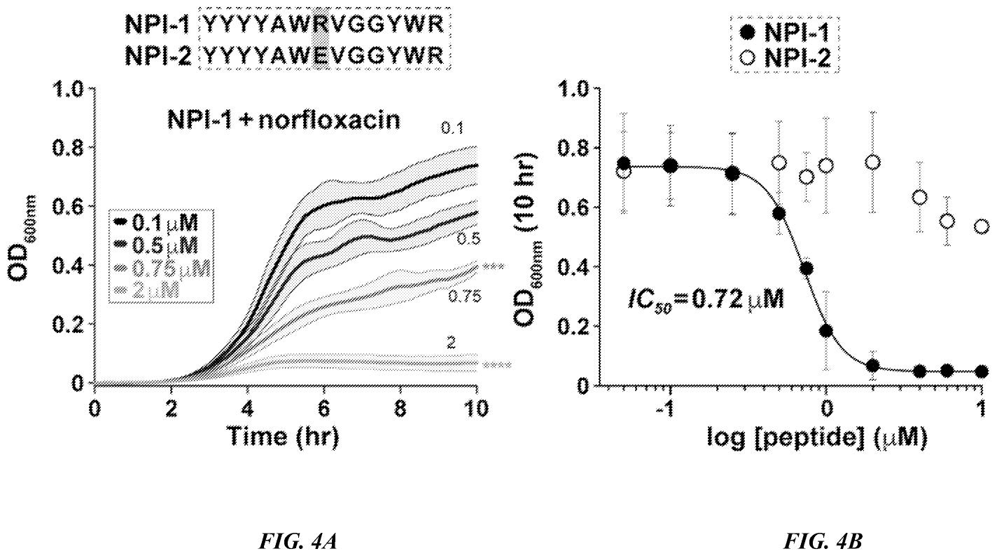

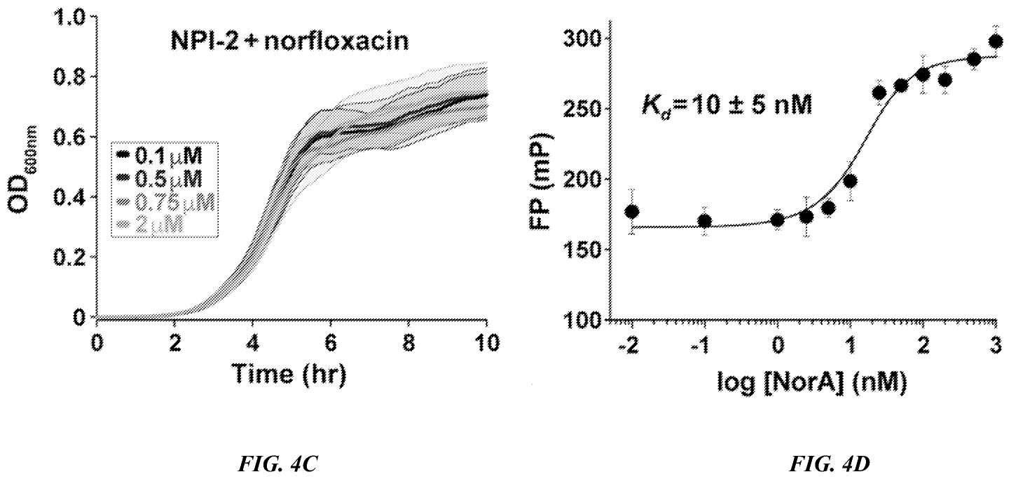

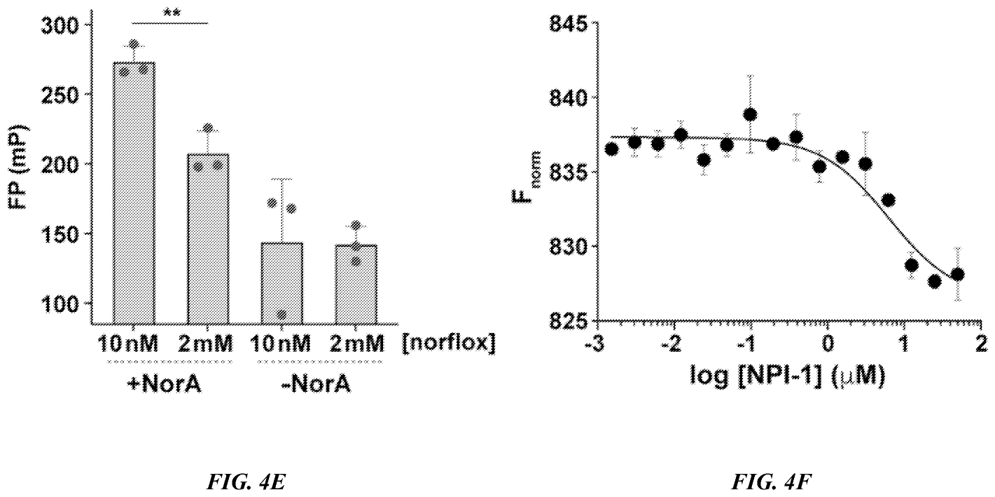

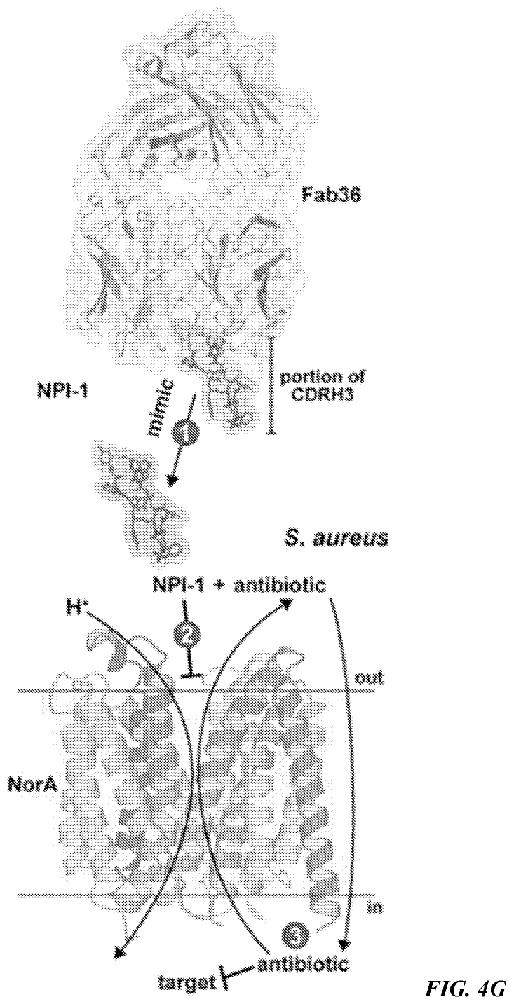

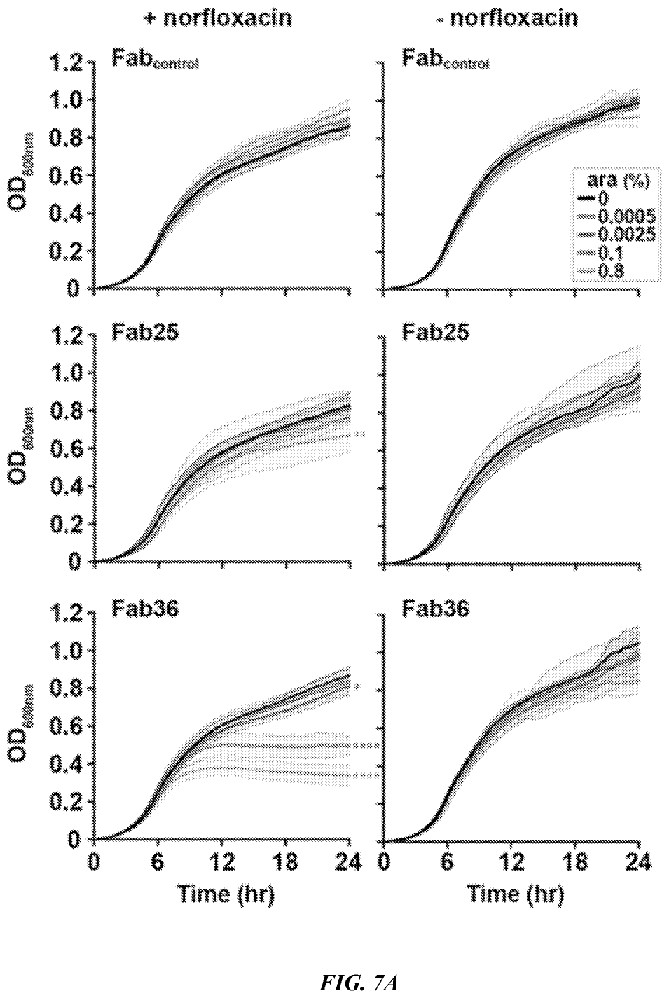

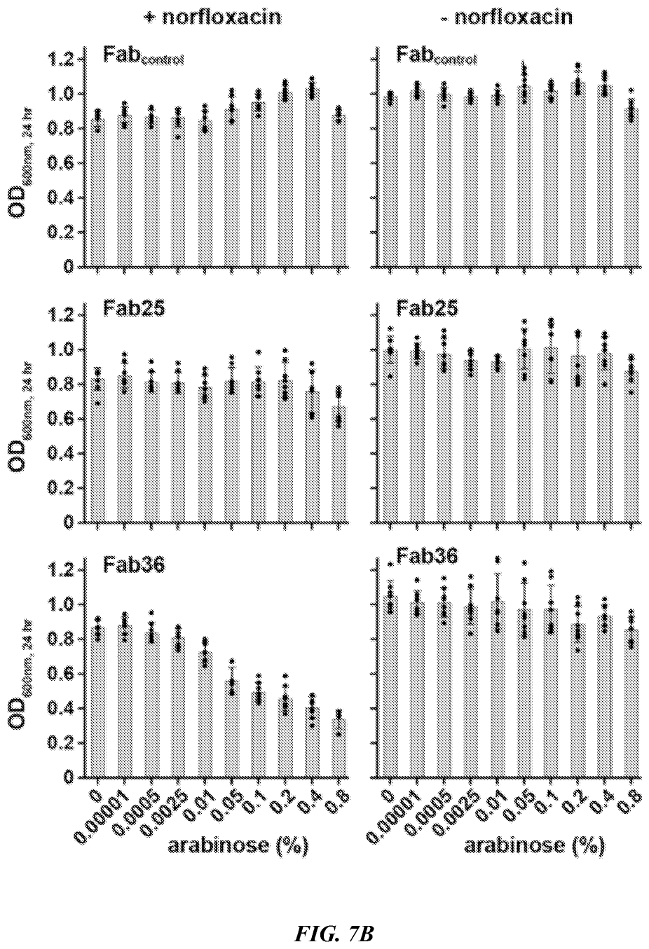

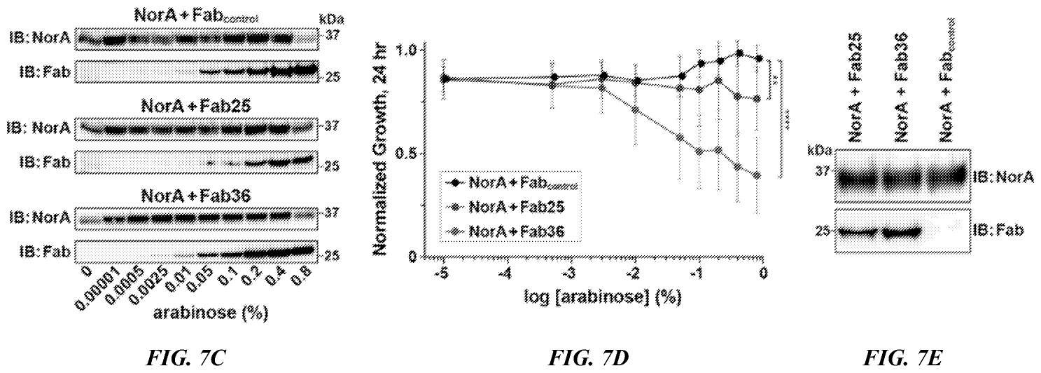

The insertion of Fab CDRH3 loops into the substrate binding pocket of NorA, as observed in the cryo-EM structures, afforded the expectation that Fab binding would inhibit antibiotic efflux (FIGS. 1A-1D, 4G). To test this hypothesis and avoid complications associated with Fab (50 kDa) permeation through the S. aureus peptidoglycan cell wall (Pasquina-Lemonche et al., “The Architecture of the Gram-positive Bacterial Cell Wall. Nature 582:294-297 (2020), which is hereby incorporated by reference in its entirety), an inhibition experiment was designed in E. coli where NorA and Fabs were co-expressed under the control of separate inducible promoters (FIG. 3A). Since NorA is located within the inner membrane and confers norfloxacin resistance to E. coli (FIG. 6D), growth inhibition was assessed by co-expressing Fabs in the periplasm to allow access to the extracellular face of NorA. Growth curves (OD600 nm vs. time) were quantified with or without norfloxacin and with variable concentrations of arabinose for inducing Fab expression. Co-expression of NorA with Fab36 in the presence of norfloxacin strongly attenuated E. coli growth in an arabinose-dependent manner (FIGS. 3B-3D). Similar experiments with Fab25 also showed inhibition of NorA, albeit to a lesser extent than for Fab36, which correlated with its poorer binding affinity to NorA (FIGS. 7A-7D). In contrast, co-expression of NorA with a negative-control Fab (Fab_{control}), containing polyserine residues in the CDR loops, showed no inhibition (FIGS. 3B-3D). Likewise, no growth inhibition for Fab25, Fab36, or Fab_{control} was observed in the absence of norfloxacin (FIGS. 7A-7B), which supports a synergistic effect on E. coli growth attenuation only when both the Fab inhibitor and antibiotic are present.

To validate that NorA inhibition resulted from direct binding by the Fabs in E. coli, a pull-down experiment was carried out by co-expressing NorA with Fabs and purifying NorA from the membrane fraction. The elution fraction of NorA co-expressed with Fab25 and Fab36 contained the Fabs as detected through immunoblotting, while no Fab was detected in the elution of NorA co-expressed with Fab_{control} (FIG. 7E). Taken together with the growth inhibition results, these data confirm that Fab25 and Fab36 can bind and inhibit NorA in E. coli. Furthermore, since the Fab_{control} was unable to bind or inhibit NorA, it was concluded that the insertion of the CDRH3 loop into the substrate binding pocket of NorA, as observed in the cryo-EM structures, was responsible for the mode of inhibition.

### Example 5—CDRH3 Peptides Potentiate Norfloxacin Activity Against MRSA

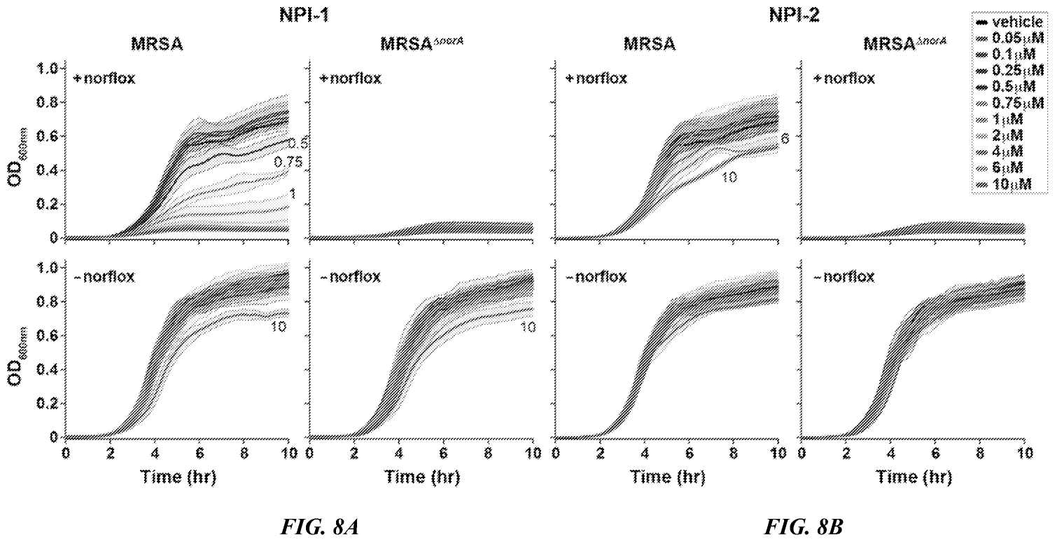

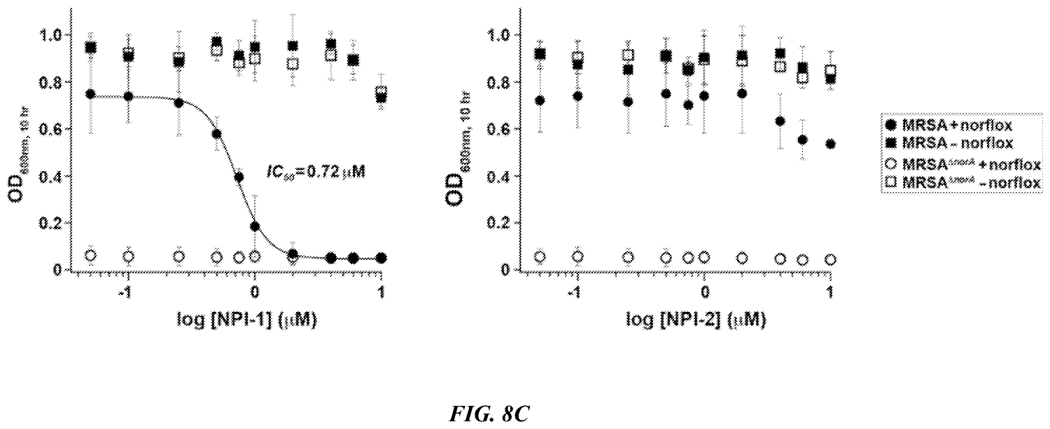

Inhibition of NorA by Fabs and the insertion of CDRH3 into the substrate binding pocket indicated that peptides mimicking the structure of the CDRH3 loops might also inhibit NorA. Such peptides would likely permeate the dense S. aureus peptidoglycan cell wall more efficiently than the 50-kDa Fabs (Pasquina-Lemonche et al., “The Architecture of the Gram-positive Bacterial Cell Wall. Nature 582:294-297 (2020), which is hereby incorporated by reference in its entirety). Since Fab36 displayed better binding affinity and inhibition to NorA than Fab25, a peptide (NorA peptide inhibitor, NPI-1) corresponding to a segment of Fab36 CDRH3 (Tyr128 to Trp139) with an additional C-terminal arginine residue for solubility (FIG. 4A) was synthesized and tested for its ability to potentiate norfloxacin activity against MRSA. In the presence of norfloxacin, NPI-1 produced dose-dependent growth inhibition of MRSA with an IC₅₀ value of 0.72±0.08 μM (FIGS. 4A-4B). Notably, NPI-1 did not inhibit the growth of MRSA in the absence of norfloxacin, supporting the peptide's role as an adjuvant (FIG. 8A). Furthermore, a control peptide substituting a glutamate for the arginine residue in the tryptophan-arginine motif (NPI-2) displayed no growth arrest of MRSA in the presence of norfloxacin (FIGS. 4B-4C; 8B-8C).

To validate interactions in vitro, fluorescence polarization (FP) experiments were carried out and it was determined that NPI-1 bound to NorA with a K_{d} value of 10±5 nM (FIG. 4D). Additionally, norfloxacin was found to compete with NPI-1 binding to NorA, as revealed through a competition assay (FIG. 4E). Lastly, it was determined that NPI-1 could displace bound Fab36 from NorA (FIG. 4F) in a manner consistent with NPI-1's K_{d} value to NorA. This result supports the conclusion that NPI-1 makes similar intermolecular contacts as CDRH3 within the NorA-Fab36 complex. It is notable that NPI-1 bound NorA with ˜14-fold greater affinity than Fab36 to NorA. It is believed that this stems from NPI-1 retaining only the most critical interactions with NorA. Collectively, these data confirm NPI-1 binding to NorA in vitro and demonstrate that peptides targeting essential residues in NorA can serve as potent inhibitors of MRSA growth in combination with antibiotics (FIG. 4G).

### Discussion of Examples 1-5

The preceding Examples illustrate the use of synthetic antibodies to determine NorA-Fab structures by cryo-EM and to design a sub-micromolar inhibitor of NorA. The latter studies resulted in the creation and subsequent validation of a peptide EPI that targets a primary antibiotic resistance mechanism used by MRSA for evading fluoroquinolone antibiotics. There are at least three advantages of peptide EPIs for treating MRSA infections in combination with existing antibiotics. First, peptide EPIs can be readily modified given the tractability of peptide chemistry, facilitating improvements in affinity and reductions in off-target effects; the latter of which has impeded the development of small molecule EPIs as therapeutics. Second, peptide EPIs can penetrate through the peptidoglycan cell wall and target the extracellular surface of efflux pumps without the need to cross the cell membrane, thereby bypassing complications associated with membrane permeability. Third, peptide EPIs are expected to be less susceptible to escape mutations given their capacity to form larger interaction interfaces with efflux pumps compared to small molecules. NPI-1, for example, makes extensive intermolecular contacts with residues in NorA's substrate binding pocket that are essential for efflux activity (Asp137, Phe140, Glu222, Asp307). Therefore, an escape mechanism to nullify NPI-1 binding and inhibition would likely require two or more simultaneous mutations at positions that do not impact drug efflux activity—an unlikely scenario. For these reasons, the peptide EPI described in this work is a promising lead compound for treating MRSA infections in combination with fluoroquinolone antibiotics, which have previously been classified as ineffective due to resistance mechanisms mediated by NorA. To further validate NPI-1 and similar peptide EPIs as viable pre-clinical therapeutic candidates, additional work is needed to determine and optimize their mammalian toxicity profiles and susceptibility to host proteases. The latter will likely necessitate the use of higher stability peptidomimetics such as constrained β-hairpins (Sawyer & Arora, “Hydrogen Bond Surrogate Stabilization of beta-Hairpins,” Acs Chem Biol 13:2027-2032 (2018), which is hereby incorporated by reference in its entirety). Overcoming these immediate hurdles would establish peptide EPIs as a promising class of therapeutics for combatting antibiotic-resistant bacterial infections where current antibiotics are rendered ineffective due to drug efflux.

### Example 6—Deep Mutational Scanning of N3-36 CDR3-HC (SEQ ID NO: 7)

To identify permissible substitutions in the CDR3-HC sequence of N3-36 (SEQ ID NO: 7), deep mutational scanning was performed (Araya et al., “Deep Mutational Scanning: Assessing Protein Function on a Massive Scale,” Trends Biotechnol. 29(9):435-42 (2011); Fowler et al., “High-resolution Mapping of Protein Sequence-Function Relationships,” Nat Methods. 7(9):741-6 (2010), each of which is hereby incorporated by reference in its entirety).

First, N3-36 (SEQ ID NO: 32) was cloned in a single-chain Fv format in a yeast-surface display vector, as previously described (Boder et al., “Yeast Surface Display for Screening Combinatorial Polypeptide Libraries,” Nat Biotechnol. 15(6):553-7 (1997); Chao et al., “Isolating and Engineering Human Antibodies Using Yeast Surface Display,” Nat Protoc. 1(2):755-68 (2006); Romero et al., “High-valency Anti-CD99 Antibodies Toward the Treatment of T Cell Acute Lymphoblastic Leukemia,” J Mol Biol. 434(5):167402 (2022), each of which is hereby incorporated by reference in its entirety). Based on an inspection of the crystal structure of the interface between N3-36 and NorA described in the preceding examples (see also Brawley et al., “Structural Basis for Inhibition of the Drug Efflux Pump NorA from Staphylococcus aureus,” Nat Chem Biol. 18(7):706-12 (2022), which is hereby incorporated by reference in its entirety), residues 4-15 of SEQ ID NO: 7 were selected for this analysis because those residues make direct contacts with NorA. A combinatorial library was made by diversifying each of these positions one at a time using the NNK codon (where N is a mixture of A, T, G and C; K is a mixture of G and T) that encodes all the genetically encoded, standard 20 amino acids. The resulting library was sorted for binding to NorA embedded in biotinylated nanodiscs prepared as described in the preceding examples using fluorescence-activated cell sorting (Boder et al., “Yeast Surface Display for Screening Combinatorial Polypeptide Libraries,” Nat Biotechnol. 15(6):553-7 (1997); Chao et al., “Isolating and Engineering Human Antibodies Using Yeast Surface Display,” Nat Protoc. 1(2):755-68 (2006); Romero et al., “High-valency Anti-CD99 Antibodies Toward the Treatment of T Cell Acute Lymphoblastic Leukemia,” J Mol Biol. 434(5):167402 (2022), each of which is hereby incorporated by reference in its entirety). The amino acid sequences of clones exhibiting binding to NorA were deduced from deep sequencing, and are shown in Table 5 below.

TABLE 5
Consensus Residues for the CDR3-HC Sequence
Following Mutation of N3-36 (SEQ ID NO: 7)

| Posi- | Original | Consensus |
|---|---|---|
| tion | Amino Acid | |
| 4 | Y | Y I V |
| 5 | Y | Y F W |
| 6 | Y | Y F I L M V W |
| 7 | Y | Y W |
| 8 | A | A L S V |
| 9 | W | W |
| 10 | R | R |
| 11 | V | V A D F G H I L M R W Y |
| 12 | G | G F P S T |
| 13 | G | G A F I L N Q S T V Y |
| 14 | Y | Y A C F H I L M R S T V W |
| 15 | W | W F L Y |

The consensus sequence generated from the deep sequencing is X₁X₂X₃X₄X₅WRX₈X₉X₁₀X₁₁X₁₂ (SEQ ID NO: 27) where the X residues at each of positions 1-5 and 8-12 is shown in Table 5 (for corresponding positions 4-8 and 11-15 of SEQ ID NO: 7). The other positions that have not been mutated in this analysis, for example at positions 1-3 and 16-20 of SEQ ID NO: 7, make few direct contacts with NorA. Thus, it is expected that those residues can be omitted or changed to any other amino acid.

### SEQUENCE LISTING

The Sequence Listing is being submitted electronically in XML format and is hereby incorporated by reference in its entirety. Said XML copy, created on Aug. 5, 2022, is named 147462_002301_ST26.xml and is 59 KB in size. No new matter is being introduced.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto

What is claimed is:

1. A NorA peptide inhibitor, said peptide inhibitor comprising the amino acid sequence of X₁YYX₄X₅WRX₈X₉GX₁₁X₁₂ (SEQ ID NO:1), wherein X₁, X₄, X₅, X₈, X₉, X₁₁, X₁₂ are selected from any amino acid residue.

2. The NorA peptide inhibitor of claim 1, wherein X₁ is G or Y, X₄ is P or Y, X₅ is Y or A, X₈ is M or V, X₉ is Y or G, X₁₁ is F or Y and X₁₂ is Y or W.

3. The NorA peptide inhibitor of claim 1, wherein said peptide inhibitor comprises the amino acid sequence of YYYYAWRVGGYW (SEQ ID NO: 2).

4. The NorA peptide inhibitor of claim 1, wherein said peptide inhibitor comprises the amino acid sequence of YYYYAWRVGGYWR (SEQ ID NO: 3).

5. The NorA peptide inhibitor of claim 1, wherein said peptide inhibitor comprises the amino acid sequence of GYYPYWRMYGFY (SEQ ID NO: 4).

6. The NorA peptide inhibitor of claim 1, wherein said peptide inhibitor comprises the amino acid sequence of WETGYYPYWRMYGFYWALDY (SEQ ID NO: 6) or the amino acid sequence of YSRYYYYAWRVGGYWGGLDY (SEQ ID NO: 7).

7. A NorA peptide inhibitor, said peptide inhibitor comprising the amino acid sequence of X₁X₂X₃X₄X₅WRX₈X₉X₁₀X₁₁X₁₂ (SEQ ID NO: 27) wherein X₁ is Y, I, or V; X₂ is Y, F, or W; X₃ is Y, F, I, L, M, V, or W; X₄ is Y or W; X₅ is A, L, S, or V; X₈ is V, A, D, F, G, H, I, L, M, R, W, or Y; X₉ is G, F, P, S, or T; X₁₀ is G, A, F, I, L, N, Q, S, T, V, or Y; X₁₁ is Y, A, C, F, H, I, L, M, R, S, T, V, or W; and X₁₂ is W, F, L, or Y.

8. A protein scaffold comprising the NorA peptide inhibitor of any one of claims 1-7.

9. The protein scaffold of claim 8, wherein said scaffold is an antibody mimetic.

10. The protein scaffold of claim 9, wherein the antibody mimetic is selected from a fibronectin type III (FN3) domain scaffold (monobody), an affibody (Z-domain of protein A), an albumin-binding domain (ABD)-Derived Affinity Protein (ADAPT) (ABD of protein G), a designed ankyrin repeat protein (DARPin), an anticalin, and a fynomer.

11. The protein scaffold of claim 8, wherein said protein scaffold is an immunoglobulin molecule or fragment thereof.

12. The protein scaffold of claim 11, wherein the scaffold is a Fab, F(ab)₂, Fv, or Fc fragment of an immunoglobulin molecule.

13. The protein scaffold of claim 11, wherein the scaffold is a single-domain antibody (nanobody).

14. An antibody-based molecule that binds NorA, said antibody-based molecule comprising a heavy chain variable region, wherein said heavy chain variable region comprises:
a complementarity-determining region 1 (CDR-H1) comprising an amino acid sequence of any one of SEQ ID NOs: 12-14, or a modified amino acid sequence of any one of SEQ ID NOs: 12-14, said modified sequence having at least 80% sequence identity to any one of SEQ ID NOs: 12-14;
a complementarity-determining region 2 (CDR-H2) comprising an amino acid sequence of any one of SEQ ID NOs: 15-20, or a modified amino acid sequence of any one of SEQ ID NOs: 15-20, said modified sequences having at least 80% sequence identity to any one of SEQ ID NOs: 15-20; and/or
a complementarity-determining region 3 (CDR-H3) comprising an amino acid sequence of any one of SEQ ID NOs: 5-11, or a modified amino acid sequence of any one of SEQ ID NO: 5-11, said modified sequence having at least 80% sequence identity to any one of SEQ ID NOs: 5-11.

15. The antibody-based molecule of claim 14, wherein said antibody-based molecule comprises a heavy chain variable region selected from the group consisting of:
(i) a heavy chain variable region comprising the CDR-H1 of SEQ ID NO: 12, the CDR-H2 of SEQ ID NO: 15, and the CDR-H3 of SEQ ID NO: 5 (N3-24);
(ii) a heavy chain variable region comprising the CDR-H1 of SEQ ID NO: 13, the CDR-H2 of SEQ ID NO: 16, and the CDR-H3 of SEQ ID NO: 6 (N3-25);
(iii) a heavy chain variable region comprising the CDR-H1 of SEQ ID NO: 13, the CDR-H2 of SEQ ID NO: 16, and the CDR-H3 of SEQ ID NO: 7 (N3-36);
(iv) a heavy chain variable region comprising the CDR-H1 of SEQ ID NO: 13, the CDR-H2 of SEQ ID NO: 17, and the CDR-H3 of SEQ ID NO: 8 (N3-38);
(v) a heavy chain variable region comprising the CDR-H1 of SEQ ID NO: 13, the CDR-H2 of SEQ ID NO: 18, and the CDR-H3 of SEQ ID NO: 9 (N3-39);
(vi) a heavy chain variable region comprising the CDR-H1 of SEQ ID NO: 13, the CDR-H2 of SEQ ID NO: 19, and the CDR-H3 of SEQ ID NO: 10 (N3-41); and
(vii) a heavy chain variable region comprising the CDR-H1 of SEQ ID NO: 14, the CDR-H2 of SEQ ID NO: 20, and the CDR-H3 of SEQ ID NO: 11 (N3-45).

16. The antibody-based molecule of claim 14 or claim 15, wherein said antibody-based molecule is a single-domain antibody (nanobody).

17. The antibody-based molecule of any one of claims 14-16, wherein said heavy chain variable region of said antibody-based molecule further comprises human immunoglobulin heavy chain framework regions.

18. The antibody-based molecule of any one of claims 14-17, wherein said antibody-based molecule comprises:
(i) a heavy chain variable region comprising an amino acid sequence that is at least 80% identical to SEQ ID NO: 28;
(ii) a heavy chain variable region comprising an amino acid sequence that is at least 80% identical to SEQ ID NO: 30;
(iii) a heavy chain variable region comprising an amino acid sequence that is at least 80% identical to SEQ ID NO: 32;
(iv) a heavy chain variable region comprising an amino acid sequence that is at least 80% identical to SEQ ID NO: 34;
(v) a heavy chain variable region comprising an amino acid sequence that is at least 80% identical to SEQ ID NO: 36;
(vi) a heavy chain variable region comprising an amino acid sequence that is at least 80% identical to SEQ ID NO: 38; and
(vii) a heavy chain variable region comprising an amino acid sequence that is at least 80% identical to SEQ ID NO: 40.

19. The antibody-based molecule of claim 14 or claim 15, wherein said antibody-based molecule further comprises a light chain variable region, wherein said light chain variable region comprises:
a complementarity-determining region 1 (CDR-L1) having an amino acid sequence of SEQ ID NOs: 21, or a modified amino acid sequence of SEQ ID NO: 21, said modified sequence having at least 80% sequence identity to SEQ ID NO: 21;
a complementarity-determining region 2 (CDR-L2) having an amino acid sequence of SEQ ID NO: 22, or a modified amino acid sequence of SEQ ID NO: 22, said modified sequence having at least 80% sequence identity to SEQ ID NO: 22; and
a complementarity-determining region 3 (CDR-L3) having an amino acid sequence of any one of SEQ ID NOs: 23-27, or a modified amino acid sequence of any one of SEQ ID NO: 23-27, said modified sequence having at least 80% sequence identity to any one of SEQ ID NO: 23-27.

20. The antibody-based molecule of claim 19, wherein said light chain variable region is selected from the group consisting of:
(i) a light chain variable region comprising the CDR-L1 of SEQ ID NO: 21, the CDR-L2 of SEQ ID NO: 22, and the CDR-L3 of SEQ ID NO: 23;
(ii) a light chain variable region comprising the CDR-L1 of SEQ ID NO: 21, the CDR-L2 of SEQ ID NO: 22, and the CDR-L3 of SEQ ID NO: 24;
(iii) a light chain variable region comprising the CDR-L1 of SEQ ID NO: 21, the CDR-L2 of SEQ ID NO: 22, and the CDR-L3 of SEQ ID NO: 25; and
(iv) a light chain variable region comprising the CDR-L1 of SEQ ID NO: 21, the CDR-L2 of SEQ ID NO: 22, and the CDR-L3 of SEQ ID NO: 26.

21. The antibody-based molecule of claim 20, wherein said light chain variable region of said antibody-based molecule further comprises human immunoglobulin light chain framework regions.

22. The antibody-based molecule of any one of claims 19-21, wherein said antibody-based molecule comprises:
(i) a light chain variable region comprising an amino acid sequence that is at least 80% identical to SEQ ID NO: 29;
(ii) a light chain variable region comprising an amino acid sequence that is at least 80% identical to SEQ ID NO: 31;
(iii) a light chain variable region comprising an amino acid sequence that is at least 80% identical to SEQ ID NO: 33;
(iv) a light chain variable region comprising an amino acid sequence that is at least 80% identical to SEQ ID NO: 35;
(v) a light chain variable region comprising an amino acid sequence that is at least 80% identical to SEQ ID NO: 37;
(vi) a light chain variable region comprising an amino acid sequence that is at least 80% identical to SEQ ID NO: 39; and
(vii) a light chain variable region comprising an amino acid sequence that is at least 80% identical to SEQ ID NO: 41.

23. The antibody-based molecule of any one of claims 14-22, wherein said antibody-based molecule comprises:
(i) a heavy chain variable region comprising the CDR-H1 of SEQ ID NO: 12, the CDR-H2 of SEQ ID NO: 15, and the CDR-H3 of SEQ ID NO: 5, and a light chain variable region comprising the CDR-L1 of SEQ ID NO: 21, the CDR-L2 of SEQ ID NO: 22, and the CDR-L3 of SEQ ID NO: 23 (N3-24);
(ii) a heavy chain variable region comprising the CDR-H1 of SEQ ID NO: 13, the CDR-H2 of SEQ ID NO: 16, and the CDR-H3 of SEQ ID NO: 6, and a light chain variable region comprising the CDR-L1 of SEQ ID NO: 21, the CDR-L2 of SEQ ID NO: 22, and the CDR-L3 of SEQ ID NO: 24 (N3-25);
(iii) a heavy chain variable region comprising the CDR-H1 of SEQ ID NO: 13, the CDR-H2 of SEQ ID NO: 16, and the CDR-H3 of SEQ ID NO: 7, and a light chain variable region comprising the CDR-L1 of SEQ ID NO: 21, the CDR-L2 of SEQ ID NO: 22, and the CDR-L3 of SEQ ID NO: 24 (N3-36);
(iv) a heavy chain variable region comprising the CDR-H1 of SEQ ID NO: 13, the CDR-H2 of SEQ ID NO: 17, and the CDR-H3 of SEQ ID NO: 8, and a light chain variable region comprising the CDR-L1 of SEQ ID NO: 21, the CDR-L2 of SEQ ID NO: 22, and the CDR-L3 of SEQ ID NO: 25 (N3-38);
(iv) a heavy chain variable region comprising the CDR-H1 of SEQ ID NO: 13, the CDR-H2 of SEQ ID NO: 18, and the CDR-H3 of SEQ ID NO: 9, and a light chain variable region comprising the CDR-L1 of SEQ ID NO: 21, the CDR-L2 of SEQ ID NO: 22, and the CDR-L3 of SEQ ID NO: 24 (N3-39);
(iv) a heavy chain variable region comprising the CDR-H1 of SEQ ID NO: 13, the CDR-H2 of SEQ ID NO: 19, and the CDR-H3 of SEQ ID NO: 10, and a light chain variable region comprising the CDR-L1 of SEQ ID NO: 21, the CDR-L2 of SEQ ID NO: 22, and the CDR-L3 of SEQ ID NO: 24 (N3-41); and
(iv) a heavy chain variable region comprising the CDR-H1 of SEQ ID NO: 14, the CDR-H2 of SEQ ID NO: 20, and the CDR-H3 of SEQ ID NO: 11, and a light chain variable region comprising the CDR-L1 of SEQ ID NO: 21, the CDR-L2 of SEQ ID NO: 22, and the CDR-L3 of SEQ ID NO: 26 (N3-45).

24. The antibody-based molecule of claim 23, wherein said antibody-based molecule comprises:
(i) a heavy chain variable region comprising an amino acid sequence that is at least 80% identical to SEQ ID NO: 28 and a light chain variable region comprising an amino acid sequence that is at least 80% identical to SEQ ID NO: 29 (N3-24);
(ii) a heavy chain variable region comprising an amino acid sequence that is at least 80% identical to SEQ ID NO: 30 and a light chain variable region comprising an amino acid sequence that is at least 80% identical to SEQ ID NO: 31 (N3-25);
(iii) a heavy chain variable region comprising an amino acid sequence that is at least 80% identical to SEQ ID NO: 32 and a light chain variable region comprising an amino acid sequence that is at least 80% identical to SEQ ID NO: 33 (N3-36);
(iv) a heavy chain variable region comprising an amino acid sequence that is at least 80% identical to SEQ ID NO: 34 and a light chain variable region comprising an amino acid sequence that is at least 80% identical to SEQ ID NO: 35 (N3-38);
(iv) a heavy chain variable region comprising an amino acid sequence that is at least 80% identical to SEQ ID NO: 36 and a light chain variable region comprising an amino acid sequence that is at least 80% identical to SEQ ID NO: 37 (N3-39);
(iv) a heavy chain variable region comprising an amino acid sequence that is at least 80% identical to SEQ ID NO: 38 and a light chain variable region comprising an amino acid sequence that is at least 80% identical to SEQ ID NO: 39 (N3-41); and
(iv) a heavy chain variable region comprising an amino acid sequence that is at least 80% identical to SEQ ID NO: 40 and a light chain variable region comprising an amino acid sequence that is at least 80% identical to SEQ ID NO: 41 (N3-45).

25. The antibody-based molecule of any one of claims 14-24, wherein said antibody-based molecule is a monoclonal antibody or binding fragment thereof.

26. The antibody-based molecule of any one of claims 14-24, wherein said antibody-based molecule is a full-length antibody, an epitope-binding fragment of an antibody, or an antibody derivative.

27. The antibody-based molecule of claim 26, wherein said antibody-based molecule is an epitope binding fragment selected from a F(ab) fragment, a F(ab′) fragment, and F(ab′)₂ fragment.

28. The antibody-based molecule of claim 27, wherein said antibody-based molecule is a F(ab) fragment.

29. The antibody-based molecule of claim 26, where said antibody-based molecule is an antibody derivative selected from the group consisting of a scFv, a minibody, a diabody, a triabody, a tribody and a tetrabody.

30. An isolated polynucleotide encoding the NorA peptide inhibitor of any one of claims 1-7, the protein scaffold of any one of claims 8-13, or the antibody-based molecule of any one of claims 14-29.

31. The isolated polynucleotide of claim 30, wherein said polynucleotide is a messenger RNA (mRNA) molecule

32. A vector comprising the isolated polynucleotide of claim 30 or claim 31.

33. A delivery vehicle comprising the isolated polynucleotide of claim 30 or claim 31 or the vector of claim 32.

34. The delivery vehicle of claim 33, wherein said vehicle comprises a nanoparticle delivery vehicle, a lipid-based particle delivery vehicle, or a polymer-based particle delivery vehicle.

35. The delivery vehicle of claim 34, wherein said delivery vehicle is a nanoparticle delivery vehicle selected from gold nanoparticles, calcium phosphate nanoparticles, cadmium (quantum dots) nanoparticles, and iron oxide nanoparticles.

36. The delivery vehicle of claim 34, wherein said delivery vehicle is a lipid-based particle delivery vehicle selected from cationic lipid based lipoplexes (e.g., 1,2-dioleoyl-3trimethylammonium-propane (DOTAP)), neutral lipid based lipoplexes (e.g., cholesterol and dioleoylphosphatidyl ethanolamine (DOPE)), anionic lipid based lipoplexes (e.g., cholesteryl hemisuccinate (CHEMS)), and pH-sensitive lipid lipoplexes (e.g., 2,3-dioleyloxy-N-[2(sperminecarboxamido)ethyl]-N,N-dimethyl-1-propanaminium trifluoroacetate (DOSPA)).

37. The delivery vehicle of claim 34, wherein said delivery vehicle is a polymer-based particle delivery vehicle comprising cationic polymers and cationic polymer conjugates, wherein said cationic polymers are selected from polyethylenimine (PEI), poly-L-lysine (PLL), polyacrylic acid (PAA), polyamideamine-epichlorohydrin (PAE), poly[2-(dimethylamino)ethyl methacrylate] (PDMAEMA), chitosan, poly(lactic-co-glycolic acid) (PLGA), gelatin, dextran, cellulose, and cyclodextrin.

38. A host cell comprising the vector of claim 32.

39. A pharmaceutical composition comprising:
the NorA peptide inhibitor of any one of claims 1-7, the protein scaffold of any one of claims 8-13, the NorA antibody-based molecule of any one of claims 14-29, the polynucleotide of claims 30-31, the vector of claim 32, or the delivery vehicle of any one of claims 33-37; and
a pharmaceutically acceptable carrier.

40. A combination therapeutic comprising:
an antibiotic and/or biocide and
a NorA inhibitor selected from the NorA peptide inhibitor of any one of claims 1-7, the protein scaffold of any one of claims 8-13, and the NorA antibody-based molecule of any one of claims 14-29.

41. The combination therapeutic of claim 40, wherein the antibiotic and/or biocide and NorA inhibitor are formulated in a single composition.

42. The combination therapeutic of claim 40, wherein the antibiotic and/or biocide and NorA inhibitor are formulated as separate compositions.

43. The combination therapeutic of any one of claims 40-42, wherein the antibiotic and/or biocide is selected from the group consisting of a fluoroquinolone, a quaternary ammonium compound, chloramphenicol, and cetrimide.

44. The combination therapeutic of claim 43, wherein the antibiotic is a fluoroquinolone selected from the group consisting of norfloxacin, enoxacin, ciprofloxacin, difloxacin, fleroxacin, lomefloxacin, pefloxacin, sparfloxacin, temafloxacin, tosufloxacin, levofloxacin, moxifloxacin, ofloxacin, gemifloxacin, and delafloxacin.

45. The combination therapeutic of any one of claims 40-44, wherein the NorA inhibitor is the NorA peptide inhibitor comprising the amino acid sequence of X₁YYX₄X₅WRX₈X₉GX₁₁X₁₂ (SEQ ID NO:1), wherein X₁, X₄, X₅, X₈, X₉, X₁₁, X₁₂ are selected from any amino acid residue.

46. The combination therapeutic of claim 45, wherein the NorA peptide inhibitor comprises an amino acid sequence selected from SEQ ID NO 2, SEQ ID NO: 3, and SEQ ID NO: 4.

47. The combination therapeutic of any one of claims 40-44, wherein the NorA inhibitor is the NorA peptide inhibitor comprising the amino acid sequence of X₁X₂X₃X₄X₅WRX₈X₉X₁₀X₁₁X₁₂ (SEQ ID NO: 27) wherein X₁ is Y, I, or V; X₂ is Y, F, or W; X₃ is Y, F, I, L, M, V, or W; X₄ is Y or W; X₅ is A, L, S, or V; X₈ is V, A, D, F, G, H, I, L, M, R, W, or Y; X₉ is G, F, P, S, or T; X₁₀ is G, A, F, I, L, N, Q, S, T, V, or Y; X₁₁ is Y, A, C, F, H, I, L, M, R, S, T, V, or W; and X₁₂ is W, F, L, or Y.

48. A method of treating a Staphylococcus aureus infection in a subject, said method comprising:
administering, to a subject having a S. aureus infection, an antibiotic or biocide and the pharmaceutical composition of claim 39, wherein said pharmaceutical composition is administered in an amount effective to treat the S. aureus infection in the subject.

49. A method of potentiating the therapeutic efficacy of an antibiotic or biocide in a subject having a Staphylococcus aureus infection, said method comprising:
administering, to a subject having a S. aureus infection and exhibiting NorA-mediated antibiotic or biocide resistance, an antibiotic or biocide and the pharmaceutical composition of claim 39, wherein the pharmaceutical compositions is administered in an amount effective to potentiate the therapeutic efficacy of the antibiotic or biocide in the subject.

50. The method of claim 48 or claim 49, wherein the antibiotic or biocide and pharmaceutical composition are administered concurrently

51. The method of claim 48 or claim 49, wherein the antibiotic or biocide and pharmaceutical composition are administered sequentially.

52. The method of claim 48 or claim 49, wherein the S. aureus infection is a methicillin-resistant or a methicillin-sensitive S. aureus infection.

53. The method of claim 48 or claim 49, wherein a lower dose of antibiotic or biocide is administered to said subject as compared to when the antibiotic or biocide is administered as a monotherapy.

54. The method of claim 48 or claim 49, wherein the antibiotic or biocide is selected from the group consisting of a fluoroquinolone, a quaternary ammonium compound, chloramphenicol, and cetrimide.

55. The method of claim 54, wherein the antibiotic is a fluoroquinolone selected from norfloxacin, enoxacin, ciprofloxacin, difloxacin, fleroxacin, lomefloxacin, pefloxacin, sparfloxacin, temafloxacin, tosufloxacin, levofloxacin, moxifloxacin, ofloxacin, gemifloxacin, and delafloxacin.

56. The method of claim 48 or claim 49, wherein the pharmaceutical composition comprises a NorA peptide inhibitor.

57. The method of claim 56, wherein the NorA peptide inhibitor comprises the amino acid sequence of X₁YYX₄X₅WRX₈X₉GX₁₁X₁₂ (SEQ ID NO:1), wherein X₁, X₄, X₅, X₈, X₉, X₁₁, X₁₂ are selected from any amino acid residue.

58. The method of claim 57, wherein the NorA peptide inhibitor comprises an amino acid sequence selected from SEQ ID NO 2, SEQ ID NO: 3, and SEQ ID NO: 4.

59. The method of claim 56, wherein the NorA peptide inhibitor comprise the amino acid sequence of X₁X₂X₃X₄X₅WRX₈X₉X₁₀X₁₁X₁₂ (SEQ ID NO: 27) wherein X₁ is Y, I, or V; X₂ is Y, F, or W; X₃ is Y, F, I, L, M, V, or W; X₄ is Y or W; X₅ is A, L, S, or V; X₈ is V, A, D, F, G, H, I, L, M, R, W, or Y; X₉ is G, F, P, S, or T; X₁₀ is G, A, F, I, L, N, Q, S, T, V, or Y; X₁₁ is Y, A, C, F, H, I, L, M, R, S, T, V, or W; and X₁₂ is W, F, L, or Y.

60. The method of any one of claims 48-59 further comprising repeating said administering.

61. The method of any one of claims 48-59, wherein said subject is a human.

62. The method of any one of claims 48-59, wherein said subject is an animal.

63. The method of claim 62, wherein the animal is a domesticated pet.

64. The method of claim 62, wherein the animal is livestock.

65. A method of diagnosing a S. aureus infection in a subject, said method comprising:
contacting a sample from a subject having a S. aureus infection with the NorA peptide inhibitor of any one of claims 1-7, the protein scaffold of any one of claims 8-13, or the NorA antibody-based molecule of any one of claims 14-29, under conditions effective to detect NorA expression in the sample;
detecting NorA expression in said sample based on said contacting; and
diagnosing the S. aureus infection in the subject based on said detecting.

66. The method of claim 65, wherein the NorA peptide inhibitor of any one of claims 1-7, the protein scaffold of any one of claims 8-13, or the NorA antibody-based molecule of any one of claims 14-29 is coupled to a detectable label.

67. The method of claim 65, wherein a treatment resistance form of S. aureus infection is diagnosed when high levels of NorA expression are detected in the sample.

68. The method of claim 65 further comprising:
treating the subject with an antibiotic or biocide and the pharmaceutical composition of claim 39.

69. A method of detecting NorA in a non-clinical biological sample, said method comprising:
contacting the sample with the NorA peptide inhibitor of any one of claims 1-7, the protein scaffold of any one of claims 8-13, or the NorA antibody-based molecule of any one of claims 14-29, under conditions effective to detect NorA expression in the sample, and
detecting NorA expression in said sample based on said contacting.

70. The method of claim 69 further comprising:
quantifying levels of NorA expression in the sample based on said detecting.

71. The method of claim 69, wherein the NorA peptide inhibitor of any one of claims 1-7, the protein scaffold of any one of claims 8-13, or the NorA antibody-based molecule of any one of claims 14-29 is coupled to a detectable label.


SEQUENCE LISTING (61 sequences)

SEQ ID NO: 1 - protein, 12 aa, unidentified
  VAR_SEQ 1: note = X at position 1 can be any amino acid, preferably Gly or Tyr
  VAR_SEQ 4: note = X at position 4 can be any amino acid, preferably Pro or Tyr
  VAR_SEQ 5: note = X at position 5 can be any amino acid, preferably Tyr or Ala
  VAR_SEQ 8: note = X at position 8 can be any amino acid, preferably Met or Val
  VAR_SEQ 9: note = X at position 9 can be any amino acid, preferably Tyr or Gly
  VAR_SEQ 11: note = X at position 11 can be any amino acid, preferably Phe or Tyr
  VAR_SEQ 12: note = X at position 12 can be any amino acid, preferably Tyr or Trp
  REGION 1..12: note = NorA peptide inhibitor consensus
XYYXXWRXXGXX

SEQ ID NO: 2 - protein, 12 aa, unidentified
  REGION 1..12: note = NorA peptide inhibitor
YYYYAWRVGGYW

SEQ ID NO: 3 - protein, 13 aa, unidentified
  REGION 1..13: note = NorA peptide inhibitor designated NPI-1
YYYYAWRVGGYWR

SEQ ID NO: 4 - protein, 12 aa, unidentified
  REGION 1..12: note = NorA peptide inhibitor
GYYPYWRMYGFY

SEQ ID NO: 5 - protein, 21 aa, unidentified
  REGION 1..21: note = NorA Antibody N3-24, Heavy Chain CDR-3
QSWHYTYFRYSSYYYQGALDY

SEQ ID NO: 6 - protein, 20 aa, unidentified
  REGION 1..20: note = NorA Antibody N3-25, Heavy Chain CDR-3
WETGYYPYWRMYGFYWALDY

SEQ ID NO: 7 - protein, 20 aa, unidentified
  REGION 1..20: note = NorA Antibody N3-36, Heavy Chain CDR-3
YSRYYYYAWRVGGYWGGLDY

SEQ ID NO: 8 - protein, 20 aa, unidentified
  REGION 1..20: note = NorA Antibody N3-38, Heavy Chain CDR-3
MYWWWYWISASGFSWYALDY

SEQ ID NO: 9 - protein, 21 aa, unidentified
  REGION 1..21: note = NorA Antibody N3-39, Heavy Chain CDR-3
SDYYYKWYYSWGWYSYYAIDY

SEQ ID NO: 10 - protein, 19 aa, unidentified
  REGION 1..19: note = NorA Antibody N3-41, Heavy Chain CDR-3
YRSYVYGYWYWYWYWAMDY

SEQ ID NO: 11 - protein, 21 aa, unidentified
  REGION 1..21: note = NorA Antibody N3-45, Heavy Chain CDR-3
SGPYRNFYFHYISMYTVALDY

SEQ ID NO: 12 - protein, 7 aa, unidentified
  REGION 1..7: note = NorA Antibody N3-24, Heavy Chain CDR-1
ISSSSIH

SEQ ID NO: 13 - protein, 5 aa, unidentified
  REGION 1..5: note = NorA Antibodies N3-25, N3-36, N3-38, N3-39, and N3-41, Heavy Chain CDR-1
SSSIH

SEQ ID NO: 14 - protein, 5 aa, unidentified
  REGION 1..5: note = NorA Antibody N3-45, Heavy Chain CDR-1
SYSIH

SEQ ID NO: 15 - protein, 17 aa, unidentified
  REGION 1..17: note = NorA Antibody N3-24, Heavy Chain CDR-2
SIYSYSGSTSYADSVKG

SEQ ID NO: 16 - protein, 17 aa, unidentified
  REGION 1..17: note = NorA Antibodies N3-25 and N3-36, Heavy Chain CDR-2
SISSSSGSTSYADSVKG

SEQ ID NO: 17 - protein, 17 aa, unidentified
  REGION 1..17: note = NorA Antibody N3-38, Heavy Chain CDR-2
SISSSYGSTSYADSVKG

SEQ ID NO: 18 - protein, 17 aa, unidentified
  REGION 1..17: note = NorA Antibody N3-39, Heavy Chain CDR-2
SISPYSGYTSYADSVKG

SEQ ID NO: 19 - protein, 17 aa, unidentified
  REGION 1..17: note = NorA Antibody N3-41, Heavy Chain CDR-2
SISSSSGSTSYADSVKG

SEQ ID NO: 20 - protein, 17 aa, unidentified
  REGION 1..17: note = NorA Antibody N3-45, Heavy Chain CDR-2
SIYSSSGSTYYADSVKG

SEQ ID NO: 21 - protein, 11 aa, unidentified
  REGION 1..11: note = NorA Antibodies N3-24, N3-25, N3-36, N3-38, N3-39, N3-41, N3-45, Light Chain CDR-1
RASQSVSSAVA

SEQ ID NO: 22 - protein, 7 aa, unidentified
  REGION 1..7: note = NorA Antibodies N3-24, N3-25, N3-36, N3-38, N3-39, N3-41, N3-45, Light Chain CDR-2
SASSLYS

SEQ ID NO: 23 - protein, 10 aa, unidentified
  REGION 1..10: note = NorA Antibody N3-24, Light Chain CDR-3
QQSYYGHLIT

SEQ ID NO: 24 - protein, 9 aa, unidentified
  REGION 1..9: note = NorA Antibodies N3-25, N3-36, N3-39, N3-41 Light Chain CDR-3
QQSSSSLIT

SEQ ID NO: 25 - protein, 10 aa, unidentified
  REGION 1..10: note = NorA Antibody N3-38, Light Chain CDR-3
QQSYYPKPFT

SEQ ID NO: 26 - protein, 10 aa, unidentified
  REGION 1..10: note = NorA Antibody N3-45, Light Chain CDR-3
QQYYFYPLIT

SEQ ID NO: 27 - skipped

SEQ ID NO: 28 - protein, 130 aa, unidentified
  REGION 1..130: note = NorA Antibody N3-24, Heavy Chain Variable Region
EVQLVESGGGLVQPGGSLRLSCAASGFTISSSSIHWVRQAPGKGLEWVASIYSYSGSTSYADSVKGRFTISADTSKNTAYLQMNSLRAEDTAVYYCARQSWHYTYFRYSSYYYQGALDYWGQGTLVTVSS

SEQ ID NO: 29 - protein, 111 aa, unidentified
  REGION 1..111: note = NorA Antibody N3-24, Light Chain Variable Region
DIQMTQSPSSLSASVGDRVTITCRASQSVSSAVAWYQQKPGKAPKLLIYSASSLYSGVPSRFSGSRSGTDFTLTISSLQPEDFATYYCQQSYYGHLITFGQGTKVEIKRTV

SEQ ID NO: 30 - protein, 129 aa, unidentified
  REGION 1..129: note = NorA Antibody N3-25, Heavy Chain Variable Region
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARWETGYYPYWRMYGFYWALDYWGQGTLVTVSS

SEQ ID NO: 31 - protein, 110 aa, unidentified
  REGION 1..110: note = NorA Antibody N3-25, Light Chain Variable Region
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTV

SEQ ID NO: 32 - protein, 129 aa, unidentified
  REGION 1..129: note = NorA Antibody N3-36, Heavy Chain Variable Region
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARYSRYYYYAWRVGGYWGGLDYWGQGTLVTVSS

SEQ ID NO: 33 - protein, 110 aa, unidentified; identical to SEQ ID NO: 31
  REGION 1..110: note = NorA Antibody N3-36, Light Chain Variable Region

SEQ ID NO: 34 - protein, 129 aa, unidentified
  REGION 1..129: note = NorA Antibody N3-38, Heavy Chain Variable Region
EVQLVESGGGLVQPGGSLRLSCAASGFTLYSSSIHWVRQAPGKGLEWVASISSSYGSTSYADSVKGRFTISADTSKNTAYLQMNSLRAEDTAVYYCARMYWWWYWISASGFSWYALDYWGQGTLVTVSS

SEQ ID NO: 35 - protein, 111 aa, unidentified
  REGION 1..111: note = NorA Antibody N3-38, Light Chain Variable Region
DIQMTQSPSSLSASVGDRVTITCRASQSVSSAVAWYQQKPGKAPKLLIYSASSLYSGVPSRFSGSRSGTDFTLTISSLQPEDFATYYCQQSYYPKPFTFGQGTKVEIKRTV

SEQ ID NO: 36 - protein, 130 aa, unidentified
  REGION 1..130: note = NorA Antibody N3-39, Heavy Chain Variable Region
EVQLVESGGGLVQPGGSLRLSCAASGFTFSSSSIHWVRQAPGKGLEWVASISPYSGYTSYADSVKGRFTISADTSKNTAYLQMNSLRAEDTAVYYCARSDYYYKWYYSWGWYSYYAIDYWGQGTLVTVSS

SEQ ID NO: 37 - protein, 110 aa, unidentified; identical to SEQ ID NO: 31
  REGION 1..110: note = NorA Antibody N3-39, Light Chain Variable Region

SEQ ID NO: 38 - protein, 128 aa, unidentified
  REGION 1..128: note = NorA Antibody N3-41, Heavy Chain Variable Region
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARYRSYVYGYWYWYWYWAMDYWGQGTLVTVSS

SEQ ID NO: 39 - protein, 110 aa, unidentified; identical to SEQ ID NO: 31
  REGION 1..110: note = NorA Antibody N3-41, Light Chain Variable Region

SEQ ID NO: 40 - protein, 130 aa, unidentified
  REGION 1..130: note = NorA Antibody N3-45, Heavy Chain Variable Region
EVQLVESGGGLVQPGGSLRLSCAASGFTISSYSIHWVRQAPGKGLEWVASIYSSSGSTYYADSVKGRFTISADTSKNTAYLQMNSLRAEDTAVYYCARSGPYRNFYFHYISMYTVALDYWGQGTLVTVSS

SEQ ID NO: 41 - protein, 111 aa, unidentified
  REGION 1..111: note = NorA Antibody N3-45, Light Chain Variable Region
DIQMTQSPSSLSASVGDRVTITCRASQSVSSAVAWYQQKPGKAPKLLIYSASSLYSGVPSRFSGSRSGTDFTLTISSLQPEDFATYYCQQYYFYPLITFGQGTKVEIKRTV

SEQ ID NO: 42 - protein, 13 aa, Staphylococcus aureus
  REGION 1..13: note = NorA TM7
FGLSAFETLYSLY

SEQ ID NO: 43 - protein, 13 aa, Staphylococcus aureus
  REGION 1..13: note = NorA TM10
VVFIGFDMIRPAI

SEQ ID NO: 44 - protein, 13 aa, Bacillus subtilis
  REGION 1..13: note = Bmr TM7
FGLASFESLFALF

SEQ ID NO: 45 - protein, 13 aa, Bacillus subtilis
  REGION 1..13: note = Bmr TM10
TVFVGFDLMRPAV

SEQ ID NO: 46 - protein, 13 aa, Bacillus subtilis
  REGION 1..13: note = Blt TM7
FGLSAYETVFSLF

SEQ ID NO: 47 - protein, 13 aa, Bacillus subtilis
  REGION 1..13: note = Blt TM10
FIFLAFDLLRPAL

SEQ ID NO: 48 - protein, 13 aa, Lactococcus sp.
  REGION 1..13: note = LmrP TM7
FIIMQFDNFLPVH

SEQ ID NO: 49 - protein, 13 aa, Lactococcus sp.
  REGION 1..13: note = LmrP TM10
VYTLGEIVYTPSV

SEQ ID NO: 50 - protein, 13 aa, Escherichia coli
  REGION 1..13: note = EmrD TM7
AGIAAFEACSGVL

SEQ ID NO: 51 - protein, 13 aa, Escherichia coli
  REGION 1..13: note = EmrD TM10
LFFFGAGMLFPLA

SEQ ID NO: 52 - protein, 13 aa, Escherichia coli
  REGION 1..13: note = MdfA TM7
LPLLAWIAQSPII

SEQ ID NO: 53 - protein, 13 aa, Escherichia coli
  REGION 1..13: note = MdfA TM10
IYAFGIGLANAGL

SEQ ID NO: 54 - protein, 13 aa, Escherichia coli
  REGION 1..13: note = YajR TM7
ILLMSTFVALPGQ

SEQ ID NO: 55 - protein, 13 aa, Escherichia coli
  REGION 1..13: note = YajR TM10
LFFVAFNLMEALL

SEQ ID NO: 56 - protein, 13 aa, unidentified
  REGION 1..13: note = NPI-2
YYYYAWEVGGYWR

SEQ ID NO: 57 - protein, 14 aa, unidentified
  MOD_RES 1: note = FITC-labeled beta-alanine; note = 1
  REGION 1..14: note = FITC-labeled NPI-1
XYYYYAWRVGGYWR

SEQ ID NO: 58 - protein, 13 aa, Staphylococcus aureus
  REGION 1..13: note = NorA Motif A
GTLADKLGKKLII

SEQ ID NO: 59 - protein, 13 aa, unidentified
  VAR_SEQ 2: note = X at position 2 is any amino acid
  VAR_SEQ 7: note = X at position 7 can be any amino acid
  VAR_SEQ 11..12: note = X at positions 11-12 can be any amino acid
  REGION 1..13: note = Motif A consensus
GXLADRXGRKXXI

SEQ ID NO: 60 - protein, 10 aa, Staphylococcus aureus
  REGION 1..10: note = NorA Motif C
GFILGPGIGG

SEQ ID NO: 61 - protein, 10 aa, unidentified
  VAR_SEQ 2..4: note = X at positions 2-4 can be any amino acid
  VAR_SEQ 7: note = X at position 7 can be any amino acid
  REGION 1..10: note = Motif C consensus
GXXXGPXIGG